(19) United States
(12) Patent Application Publication
(10) Pub. No.: US 2024/0055125 A1
(43) Pub. Date: Feb. 15, 2024

(54) SYSTEM AND METHOD FOR DETERMINING DATA QUALITY FOR CARDIOVASCULAR PARAMETER DETERMINATION

(71) Applicant: Riva Health, Inc., Burlingame, CA (US)

(72) Inventors: Tuhin Sinha, Burlingame, CA (US); Mark Mozolewski, Burlingame, CA (US)

(21) Appl. No.: 18/383,166

(22) Filed: Oct. 24, 2023

Related U.S. Application Data
(63) Continuation-in-part of application No. 18/224,243, filed on Jul. 20, 2023.
Application No. 18/224,243 is a continuation of application No. 17/939,773, filed on Sep. 7, 2022, now Pat. No. 11,830,624.
(60) Provisional application No. 63/241,436, filed on Sep. 7, 2021.
(60) Provisional application No. 63/419,189, filed on Oct. 25, 2022.

(51) Int. Cl. G16H 50/20; G16H 30/20; A61B 5/00; A61B 5/024; A61B 5/021

(52) U.S. Cl. CPC G16H 50/20; G16H 30/20; A61B 5/721; A61B 5/7221; A61B 5/02416; A61B 5/02108; A61B 5/6843; A61B 5/7264; G06T 7/0012

(57) ABSTRACT

The system for cardiovascular parameter data quality determination can include a user device and a computing system, wherein the user device can include one or more sensors, the computing system, and/or any suitable components. The computing system can optionally include a data quality module, a cardiovascular parameter module, a storage module, and/or any suitable modules. The method for cardiovascular parameter data quality determination can include acquiring data and determining a quality of the data. The method can optionally include processing the data, and/or determining a cardiovascular parameter, training a data quality module, any suitable steps.

### CROSS-REFERENCE TO RELATED APPLICATIONS

This application is a continuation-in-part of U.S. application Ser. No. 18/224,243 filed 20 Jul. 2023, which is a continuation of U.S. application Ser. No. 17/939,773 filed 7 Sep. 2022, which claims the benefit of U.S. Provisional Application No. 63/241,436 filed 7 Sep. 2021, each of which is incorporated in its entirety by this reference.

This application claims the benefit of U.S. Provisional Application No. 63/419,189 filed 25 Oct. 2022, which is incorporated in its entirety by this reference.

### TECHNICAL FIELD

This invention relates generally to the cardiovascular parameter field, and more specifically to a new and useful system and method in the cardiovascular parameter field.

### BRIEF DESCRIPTION OF THE FIGURES

FIG. 1A is a schematic representation of a variant of the system.

FIG. 1B is a schematic representation of an example of the system.

FIG. 2 is a schematic representation of a variant of the method.

FIG. 3 depicts an example of combining outputs of a motion model, a body region contact model, and a placement model.

FIG. 4 depicts an example of a motion model.

FIG. 5 depicts an example of a body region contact model.

FIGS. 6A, 6B, and 6C depict examples of a placement model.

FIG. 7 depicts an example of aggregating image attributes.

FIG. 8 depicts an example of generating a high quality plethysmogram (PG) dataset.

FIG. 9 depicts a first example of determining a cardiovascular parameter.

FIG. 10 depicts a second example of determining a cardiovascular parameter.

FIG. 11 depicts an example of the method.

FIG. 12 depicts an illustrative example of accumulating data segments.

FIG. 13 depicts an example of a timeseries of total luminance.

FIG. 14 depicts an example of summed luminance values rows and columns of an image.

FIG. 15 depicts an example of a timeseries of total red and blue chroma.

FIGS. 16A and 16B depict illustrative examples of using a live video to guide a user.

FIG. 17A depicts an illustrative example of guiding a user based on a motion parameter.

FIG. 17B depicts an illustrative example of guiding a user based on a contact parameter and/or a placement parameter.

FIG. 17C depicts an illustrative example of guiding a user based on a signal quality parameter (e.g., body region temperature).

FIG. 18 depicts an illustrative example of displaying a cardiovascular parameter.

FIG. 19 is a schematic representation of examples of possible fiducials determined based on a functional form fit to a segment of the PG dataset.

FIG. 20 is a schematic representation of an example of determining a linear cardiovascular manifold.

FIG. 21 is a schematic representation of an example of determining a cardiovascular parameter of a user using a universal cardiovascular manifold.

FIG. 22 is a schematic representation of an example of a transformation between cardiovascular manifolds.

FIG. 23 depicts an example of determining a data quality.

FIG. 24 is a schematic representation of an example of the system.

FIG. 25 is a schematic representation of an example of the method.

FIG. 26 depicts an example of the method.

FIG. 27 depicts an example of system modules.

FIG. 28 depicts an example of PPG signal generation.

FIG. 29 depicts an example of acquiring data using a camera.

FIG. 30 depicts an example of GPU transformation.

FIG. 31 depicts an example of user verification of manual cuff-based inputs.

FIG. 32 depicts a first example of PPG signal accumulation.

FIG. 33 depicts a second example of PPG signal accumulation.

FIG. 34 depicts an example of cardiovascular parameter calibration.

FIG. 35 depicts a first example of determining a cardiovascular parameter.

FIG. 36 depicts an example of determining a series of cardiovascular parameters.

FIG. 37 depicts a second example of determining a data quality.

FIG. 38 depicts a specific example of the system and method.

FIG. 39 depicts example components for calibration.

FIG. 40 depicts example components for cardiovascular parameter calculation.

FIG. 41 depicts example model architecture.

FIG. 42 depicts an example of extracting training from a training recording.

### DETAILED DESCRIPTION

The following description of the embodiments of the invention is not intended to limit the invention to these embodiments, but rather to enable any person skilled in the art to make and use this invention.

### 1. OVERVIEW

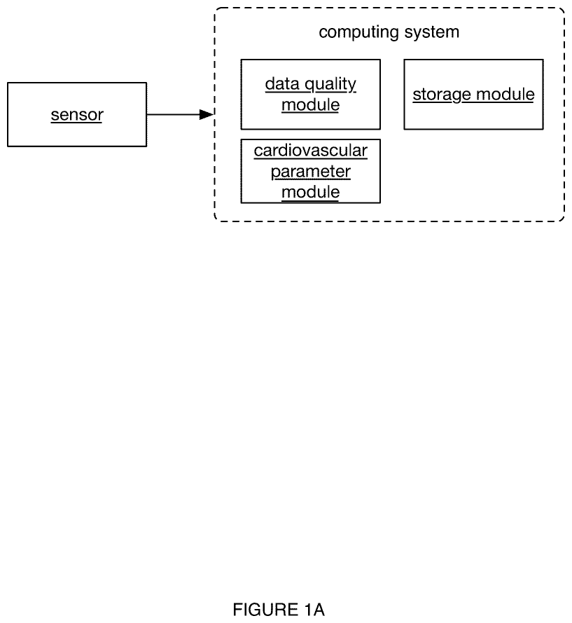

As shown in FIG. 1A, the system can include a user device and a computing system. The user device can include one or more sensors, the computing system, and/or any suitable components. The computing system can include a data quality module, a cardiovascular parameter module, a storage module, and/or any suitable module(s).

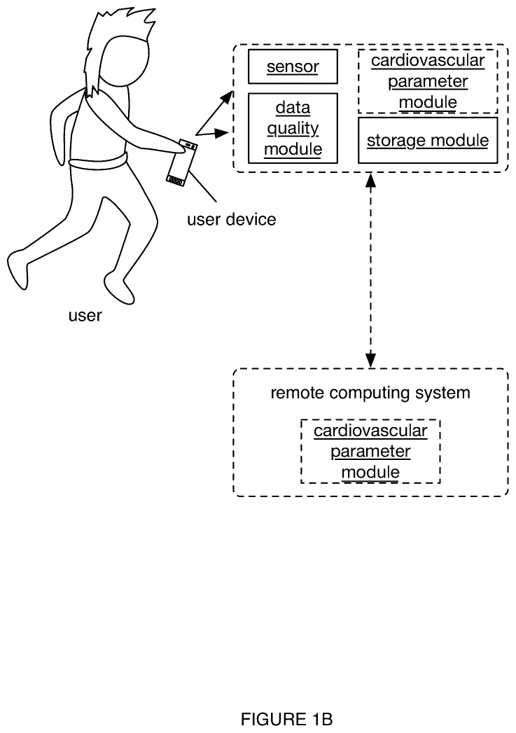

As shown in FIG. 2, the method can include acquiring data S100 and determining a quality of the data S200. The method can optionally include guiding a user based on the quality of the data S250, processing the data S300, determining a cardiovascular parameter S400, training a data quality module S500, and/or any suitable steps.

The system and method preferably function to determine a quality associated with plethysmogram data and/or determine a cardiovascular parameter based on the plethysmogram data. However, the system and method can otherwise function. Exemplary cardiovascular parameters include: blood pressure, arterial stiffness, stroke volume, heart rate, blood volume, pulse transit time, phase of constriction, pulse wave velocity, heart rate variability, blood pressure variability, medication interactions (e.g., impact of vasodilators, vasoconstrictors, etc.), cardiovascular drift, cardiac events (e.g., blood clots, strokes, heart attacks, etc.), cardiac output, cardiac index, systemic vascular resistance, oxygen delivery, oxygen consumption, baroreflex sensitivity, stress, sympathetic/parasympathetic tone, respiratory rate, blood vessel viscosity, venous function, ankle pressure, genital response, venous reflux, temperature sensitivity, and/or any suitable cardiovascular parameters and/or properties.

### 2. EXAMPLES

In an example, the system can include: a user device that includes a local computing system, a camera, a torch (e.g., flash), and a motion sensor; and a remote computing system (e.g., remote from the user device). The local computing system can include a data quality module, wherein the data quality module includes a motion model, a body region contact model, and a placement model. A cardiovascular parameter module is preferably executed by the remote computing system, but can be distributed between the local and remote computing systems and/or located on the local computing system. In this example, the method can include: a user placing their finger on the torch and a lens of the camera, acquiring a video segment via the camera and a first motion dataset via the motion sensor, extracting a set of image attributes from the video segment (e.g., attributes of the image itself, instead of attributes of a scene captured by the image), and determining a data quality associated with the video segment based on the set of image attributes and the first motion dataset. Specific examples of image attributes include: total luminance (e.g., sum of luminance across all pixels in the image); total red, green, and/or blue chroma; and summed luminance across subsets of pixels (e.g., across pixel rows and/or columns). In an illustrative example, the motion model outputs a binary classification (e.g., ‘acceptable motion’ or ‘unacceptable motion’) based on the first motion dataset; the body region contact model outputs a binary classification (e.g., ‘finger detected’ or ‘finger not detected’) based on a first subset of the image attributes (e.g., total luminance, total red chroma, and total blue chroma for each frame of the video segment); and the placement model outputs a binary classification (e.g., ‘acceptable finger placement’ or ‘unacceptable finger placement’) and/or a multiclass classification (e.g., ‘acceptable finger placement’, ‘finger pressure too high’, ‘finger pressure too low’, ‘finger too far down’, ‘finger too far up’, ‘finger too far left’, ‘finger too far right’, ‘finger motion too high’, etc.) based on a second subset of the image attributes (e.g., an array of summed row luminance and summed column luminance for each frame of the video segment). A final data quality classification for the video segment (‘high quality’ or ‘low quality’) can be determined based on a combination of the outputs of the motion model, body region contact model, and placement model, wherein all three models must indicate acceptable conditions (e.g., ‘acceptable motion’, ‘finger detected’, and ‘acceptable finger placement’) for the video segment to be classified as ‘high quality’. The cardiovascular parameter module can determine a cardiovascular parameter of the user based on PG data extracted from a video classified as ‘high quality’ (e.g., the video segment, aggregated ‘high quality’ video segments, etc.).

### 3. TECHNICAL ADVANTAGES

Variants of the technology can confer one or more advantages over conventional technologies.

First, variants of the technology can check a quality of data to be used in determining a user or patient's cardiovascular parameters, which can help ensure that the outputs (e.g., the cardiovascular parameters) are reliable and/or accurate. Based on the data quality, the data can be used in the determination or can be recollected. For example, machine learning can be used to assess or characterize a quality of the collected data.

Second, variants of the technology can be operated or operable on a user device. For example, splitting a machine learning model into submodels (e.g., a motion model, a body region contact model, and a placement model) can simplify training of the model, help avoid overfitting or underfitting of the model, enable the models to be run on a user device, and/or otherwise enable the models to be performed or operated on a user device. Additionally, or alternatively, the technology can leverage software and/or hardware enhancements to facilitate, speed up, and/or otherwise run the models.

Third, variants of the technology can increase efficiency of data quality determination. For example, a machine learning model can be efficient enough to output a data quality classification in substantially real time (e.g., concurrently) with data acquisition and/or data quality determination, wherein the real time data quality classification can enable a user device to accumulate high quality data in real time for cardiovascular parameter determination. In variants, the efficiency of data quality determination can be increased by reducing inputs to a data quality model. For example, a body region contact model can take as input (only) total luminescence, total red chroma, and total blue chroma (e.g., no green chroma), which can result in a small (e.g., minimum) amount of data for each video frame (e.g., 3 data values for each image) used to detect finger contact (e.g., contact presence and/or pressure). A placement model can take as input (only) summed luminance across each row and column of an image, which can result in a small (e.g., minimum) amount of data used to detect which portion of the camera lens is covered/uncovered by a user's finger (e.g., detecting finger position and/or finger pressure). In variants, the placement model can correct for edge cases that would go undetected when using only the body region contact model (e.g., a user with their finger covering only the torch). In examples, the models can be combined in parallel (e.g., concurrently evaluated, which can increase overall data quality evaluation speed) and/or in series (e.g., which can decrease computational resources by mitigating unnecessary model evaluation). In variants, the computational speed can be further increased by analyzing a subsample of images from the video segment (e.g., wherein the duration between analyzed frames is shorter than a threshold determined based on user movement speed).

However, further advantages can be provided by the system and method disclosed herein.

### 4. SYSTEM

As shown in FIG. 1A, the system can include a sensor and a computing system. The system can be implemented on and/or distributed between: a user device, a remote computing device (e.g., cloud, server, etc.), care-provider device (e.g., dedicated instrument, care-provider smart phone, etc.), and/or at any suitable device (e.g., an example is shown in FIG. 1B). For example, a user device can include one or more sensors, the computing system, and/or any suitable components. Exemplary user devices include: smart phones, cellular phones, smart watches, laptops, tablets, computers, smart sensors, smart rings, epidermal electronics, smart glasses, head mounted displays, smart necklaces, dedicated and/or custom devices, and/or any suitable user device (e.g., wearable computer) can be used.

The system can function to acquire plethysmogram (PG) datasets, determine a quality of the PG datasets, provide feedback for how to improve the PG datasets, determine a cardiovascular parameter based on the PG datasets, and/or can otherwise function. The system is preferably implemented on (e.g., integrated into) a user device owned or associated with the user, but can be a standalone device, distributed between devices (e.g., a sensor device and a computing system device), and/or can otherwise be implemented or distributed. The system is preferably operable by a user, but can be operable by a healthcare professional (e.g., to measure a patient's data), a caregiver, a support person, and/or by any suitable person to measure a user's (e.g., patient, individual, client, etc.) cardiovascular parameter.

The sensor(s) preferably function to acquire one or more datasets where the datasets can be used to determine, process, evaluate (e.g., determine a quality of), and/or are otherwise related to a cardiovascular parameter. The sensors are preferably integrated into the user device, but can be stand-alone sensors (e.g., wearable sensors, independent sensors, etc.), integrated into a second user device, and/or can otherwise be mounted or located.

The sensors can be hardware or software sensors. For example, a gravity sensor can be implemented as a gravimeter (e.g., a hardware sensor) and/or be determined based on accelerometer (and/or gyroscope) data (e.g., a software sensor). Exemplary sensors include: accelerometers, gyroscopes, gravity sensors (gravimeters), magnetometers (e.g., compasses, hall sensor, etc.), GNSS sensors, environmental sensors (e.g., barometers, thermometers, humidity sensors, etc.), ambient light sensors, image sensors (e.g., cameras), and/or any suitable sensors. An image sensor can optionally include a torch (e.g., camera flash element, lighting element, LED, etc.).

At least one sensor is preferably configured to be arranged relative to a body region of a user (e.g., in contact with the body region, oriented relative to the body region, etc.), but alternatively can be not connected or related to the body region, and/or can be otherwise configured relative to the body region. The body region can be a finger, wrist, arm, neck, chest, ankle, foot, toe, leg, head, face, ear, nose, and/or any other body region. When more than one sensor is used, the body region can contact any sensor, all sensors, a specified sensor, and/or no sensors.

The body region can partially or fully cover a field of view (FOV) of an image sensor, but alternatively can not cover the FOV. The body region preferably covers the image sensor such that the entire FOV of the image sensor is covered by the body region, but alternatively can cover a portion (e.g., threshold portion) of the image sensor FOV or none of the FOV. The threshold extent of FOV coverage can be between 60%-100% of the FOV or any range or value therebetween (e.g., 70%, 80%, 90%, 95%, 98%, 99%, etc.), but can alternatively be less than 60%. The sensor is preferably partially or fully in physical contact with the body region, but alternatively can be a predetermined distance from the body region (e.g., a sensor for ambient light can be not in contact with the body region) or otherwise arranged. The threshold extent of contact coverage can be between 60%-100% of the image sensor (e.g., a lens on the image sensor and/or a torch of the image sensor, a portion of a lens on the image sensor corresponding to the FOV, etc.) or any range or value therebetween (e.g., 70%, 80%, 90%, 95%, 98%, 99%, etc.), but can alternatively be less than 60%. For example, the sensor can be an image sensor including a camera element and a torch, wherein the body region is in contact with both the camera element (e.g., a lens of the camera element) and the torch.

The sensor can have a predetermined pose (e.g., including position and/or orientation) or range of poses relative to the body region, but alternatively can not have a predetermined pose relative to the body region. The orientation of the body region with respect to the sensor can include an angle between a reference axis on the body region (e.g., central axis of a finger) and a reference axis on the sensor (e.g., an axis in the plane of the image sensor lens). The system is preferably agnostic to the orientation of the body region with respect to the sensor, but alternatively the orientation can be within a threshold angle and/or be otherwise arranged. The threshold orientation can be between −180°-180° or any range or value therebetween (e.g., −90°-90°, −45°-45°, −20°-20°, −10°-10°, etc.). A reference point on the body region (e.g., a center of a fingertip) is preferably located within a threshold distance (e.g., in the plane of the image sensor lens) from a center of the sensor (e.g., a center of the image sensor lens), but can be otherwise arranged. The threshold distance can be between 0 mm-10 mm or any range or value therebetween (e.g., 5 mm, 4 mm, 3 mm, 2 mm, 1 mm, etc.), but can alternatively be greater than 10 mm. In specific examples, a threshold distance in a first direction (e.g., y-direction) can be different than a threshold distance in a second direction (e.g., x-direction).

A contact pressure (between the body region and the sensor) is preferably within a threshold pressure range as too light of a pressure can make measurements difficult and too large of a pressure can led to artifacts and inaccurate measurements. The threshold pressure range can include pressure values between 1 oz-50 oz or any range or value therebetween (e.g., 2 oz-15 oz, 3 oz-10 oz, 4 oz-10 oz, etc.), but can alternatively be less than 1 oz or greater than 50 oz. In an illustrative example, the contact pressure is approximately the weight of a smartphone. However, there can be no limits (e.g., only an upper bound, only a lower bound, no bounds) to the contact pressure. The contact pressure can be instructed (e.g., via user instructions displayed on the user device), inferred (e.g., based on FOV coverage, using the placement model, etc.), measured (e.g., using a pressure or force sensor), otherwise determined, and/or uncontrolled.

When more than one sensor is used, each sensor preferably acquires data contemporaneously or simultaneously with the other sensors, but can acquire data sequentially, interdigitated and/or in any order. Each sensor can be synchronized with or asynchronous from other sensors. The sensor rate for a sensor to acquire data can be between 10 Hz-1000 Hz or any range or value therebetween (e.g., 30 Hz-240 Hz, 60 Hz-120 Hz, etc.), but can alternatively be less than 10 Hz or greater than 1000 Hz. In general, each sensor can acquire data at a different sensor rate. In an illustrative example, a sensor used to acquire motion datasets can acquire data at a sensor rate less than a sensor rate from an image sensor (e.g., by half, 60 Hz less, 30 Hz less, etc.). However, the sensor rates can be the same, datasets can be modified (e.g., interpolated, extrapolated, culled, etc.) such that the data rates are the same, and/or the sensors can have any suitable data rates.

The datasets acquired by the sensor(s) can include PG datasets, images (e.g., image sets, intensity, chroma data, etc.), motion datasets (e.g., accelerometer data, gyroscope data, gravity vector, significant motion data, step detector data, magnetometer data, location data, etc.), image subsets (e.g., pixels, super pixels, pixel blocks, pixel rows, pixel columns, pixel sets, features, etc.), temperature datasets, pressure datasets, depth datasets (e.g., associated with images), audio datasets, and/or any suitable datasets. PG datasets are preferably photoplethymogram (PPG) datasets (sometimes referred to as photoelectric plethysmogram), but can additionally or alternatively include strain gauge plethysmograms, impedance plethysmograms, air plethysmograms, water plethysmograms, and/or any suitable plethysmograms or datasets.

Images can be 2D, 3D, and/or have any other set of dimensions. The images can be captured in: RGB, hyperspectral, multispectral, black and white, grayscale, panchromatic, IR, NIR, UV, thermal, and/or any other wavelength. The sensor can acquire images at a frame rate between 10 frames per second (FPS)-1000 FPS or any range or value therebetween (e.g., 30 FPS-1000 FPS, 50 FPS-500 FPS, greater than 60 FPS, greater than 100 FPS, greater than 120 FPS, etc.), but can alternatively acquire images at a frame rate less than 10 FPS or greater than 1000 FPS. The images can optionally be downsampled (e.g., downsampling the frame resolution for input to the data quality module and/or the cardiovascular parameter module), cropped, and/or otherwise processed.

The images can optionally be transformed. In a first example, an image is transformed based on ambient light conditions (e.g., based on ambient light measurement sampled by ambient light sensor). In a specific example, the image is transformed such that the transformed image corresponds to a target ambient light condition (e.g., wherein the target ambient light condition was used during the data quality module training via S500 methods). In a second example, an image acquired using a first sensor (e.g., a new user device make/model) is transformed such that the transformed image corresponds to a target sensor (e.g., a previous user device make/model). In a specific example, the target sensor was used during the data quality module training (e.g., via S500 methods).

One or more images (e.g., each video frame, a subset of video frames, etc.) can be decomposed into one or more channels specific to one or more of: luma and/or luminance (e.g., an amount of light that passes through, is emitted from, and/or is reflected from a particular area), chroma and/or saturation (e.g., brilliance and/or intensity of a color), hue (e.g., dominant wavelength), intensity (e.g., average of the arithmetic mean of the R, G, B channels), and/or any other parameter (e.g., a light scattering parameter including reflection, absorption, etc.).

One or more image attributes can optionally be extracted from one or more images. The image attribute is preferably a characteristic of the image itself, but can additionally or alternatively be a characteristic of the scene or subject depicted within the image. The image attributes can optionally be downsampled (e.g., to reduce data size for input to the data quality module and/or the cardiovascular parameter module). In a specific example, PG data can be an image attribute extracted from one or more images. However, PG data can be determined from other image attributes, from image features, based on light absorption characteristics, and/or otherwise determined.

An image attribute can be extracted from a set of pixels in an image. In a first embodiment, the set of pixels includes all pixels in the image. In a second embodiment, the set of pixels is a subset of the pixels in the image (e.g., an image subregion). In a first example, the subset of pixels corresponds to one or more pixel rows and/or columns (e.g., each row and/or each column, every other row and/or column, one or more rows and/or columns at an edge of the image, etc.). In a second example, the subset of pixels is a pixel block. In a third example, the subset of pixels is a super pixel. In a fourth example, the subset of pixels corresponds to a body region (e.g., a subset of pixels corresponding to a portion of a body region in a FOV of the image sensor and/or in physical contact with the image sensor). In a fifth example, the subset of pixels correspond to pixels within a predetermined image region (e.g., center region, upper right, upper left, upper middle, lower right, lower middle, lower left, right middle, left middle, etc.).

In a first variant, the image attribute can be an aggregate luminance for the set of pixels. Aggregate luminance can be a sum (e.g., total; unweighted, weighted, etc.) of luminance values, average (e.g., unweighted, weighted, etc.) luminance values, and/or any other statistical measure. In a first specific example, total luminance across an entire image (e.g., video frame) can be used to determine a data quality and/or to generate a PG dataset. In a second specific example, the aggregate luminance for one or more subsets of pixels (and/or comparison between the subsets' aggregate luminance) can indicate which portion of the image sensor FOV is covered (e.g., wherein a brighter set of pixels indicates more light leakage from ambient light and/or the torch or flash of the image sensor, which can correspond to less coverage).

In a second variant, the image attribute can be an aggregate chroma for a set of pixels. The aggregate chroma can be a sum of chroma values, average chroma values, and/or any other statistical measure. Chroma values can correspond to red chroma, blue chroma, green chroma, and/or any other hue. In a specific example, image attributes do not include green chroma. In a first example, the chroma can be aggregated across an entire image. In a second example, the chroma can be aggregated for pixel subsets (e.g., a set of rows, a set of columns, pixel blocks, etc.).

In a third variant, the image attribute can be an aggregate intensity for a set of pixels. The aggregate intensity can be a sum of intensity values, average intensity values, and/or any other statistical measure. In a first example, the intensity can be aggregated across an entire image. In a second example, the intensity can be aggregated for pixel subsets (e.g., a set of rows, a set of columns, pixel blocks, etc.).

In a fourth variant, the image attribute can be a color parameter metric for a set of pixels. For example, a model can output the color parameter metric (e.g., multiclass, binary, value, etc.) based on luminance values (and/or any other color parameter values) for all or a subset of pixels in an image. The color parameter metric can represent a pattern of color parameters (e.g., a pattern of luminance values) across the pixels in the image.

In a fifth variant, the image attribute can be a gradient, maximum value, minimum value, location of a maximum and/or minimum value, a percent of image frame, and/or any other frame-level summary for one or more color parameters (e.g., luminance, chroma, intensity, etc.).

In a sixth variant, the image attribute can be an aggregate depth for a set of pixels in an image (e.g., wherein the aggregate depth can be determined from depth values acquired from the image sensor used to acquire the image and/or a separate sensor, using optical flow, stereoscopic methods, photogrammatic methods, etc.).

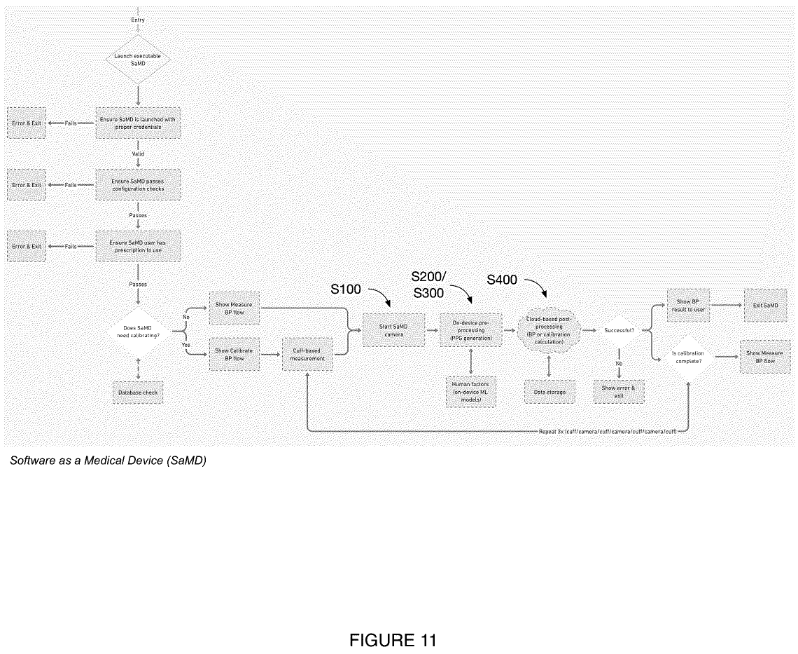

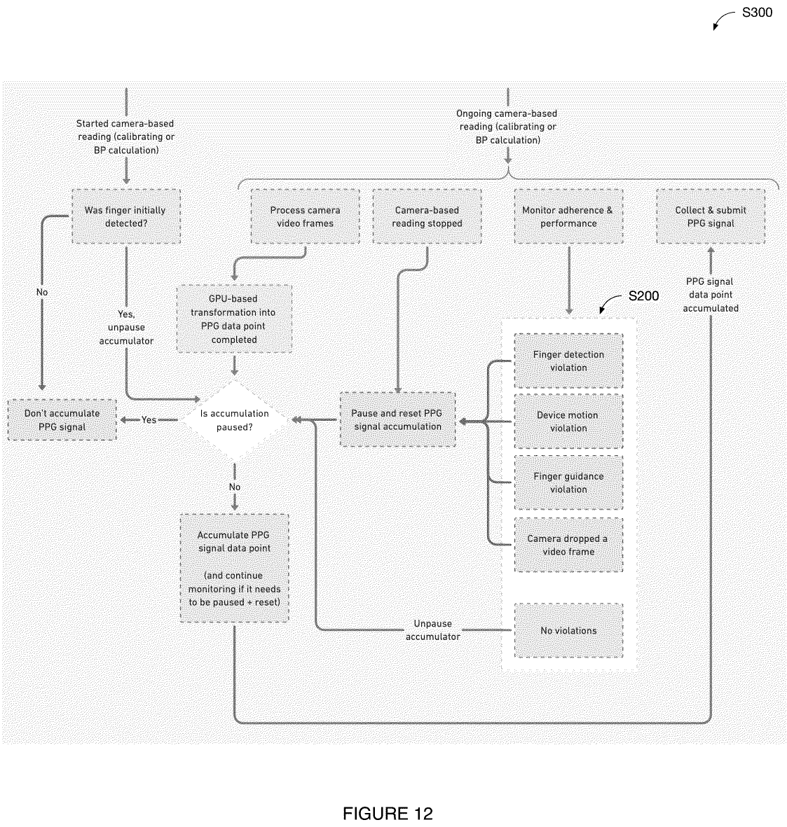

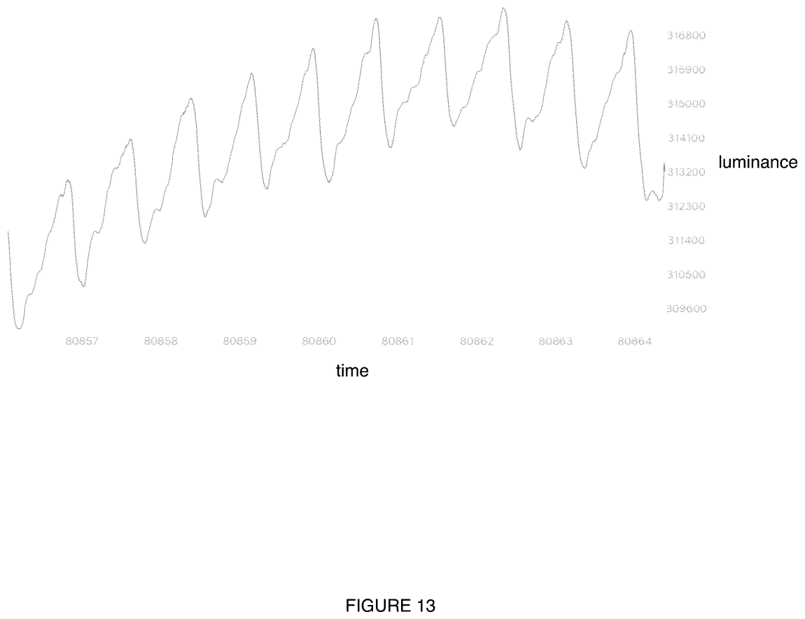

An image attribute can optionally be aggregated across a set of images (e.g., a video). In examples, the image attribute can be individually aggregated for each of a set of images (e.g., an array including a total luminance value for each frame), individually aggregated for a subset of the set of images, aggregated across the entire set of images (e.g., a single luminance value for the entire set of images), aggregated across a subset of frames, and/or otherwise aggregated. The aggregated image attribute can be: a timeseries of image attribute values (e.g., for each successive video frame), a trend (e.g., determined from the timeseries), an statistical measure (e.g., sum, min, max, mean, median, standard deviation, etc.) across the set of images (e.g., averaged attribute value from each image; an attribute determined from an average of the images, etc.), and/or be any other suitable aggregated image attribute. In a first specific example, the aggregated image attributes can include a time series of total luminance (e.g., an array including a total luminance value for each video frame); an example is shown in FIG. 13. In a second specific example, the aggregated image attributes can include a timeseries of total red chroma and/or total blue chroma; an example is shown in FIG. 15. In a third specific example, the aggregated image attributes can include a timeseries of an array of summed luminance values (e.g., luminance summed across each pixel row and each pixel column); an example is shown in FIG. 14.

However, the sensor can be otherwise configured.

The computing system preferably functions to determine the cardiovascular parameter, evaluate a quality of the datasets, process the sensor data, and/or can otherwise function. The computing system can include one or more: general purpose processors (e.g., CPU, GPU, etc.), microprocessors, accelerated processing units (APU), machine learning processors (e.g., deep learning processor, neural processing units, tensor processing units, etc.), and/or any suitable processor(s).

The computing system can include a data quality module, a cardiovascular parameter module, a storage module, and/or any suitable module(s).

The computing system can be local (e.g., integrated into the user device, a stand-alone device, etc.), remote (e.g., a cloud computing device, a server, a remote database, etc.), and/or can be distributed (e.g., between a local and a remote computing system, between one or more local computing systems, etc.). In a first specific example, the data quality module can be implemented locally on a user device (e.g., to leverage the speed of edge computing for rapid data quality analysis and/or minimize the amount of data that needs to be sent to a remote computing system) while all or parts of the cardiovascular parameter module can be implemented on a remote system. In a second specific example, the data quality module and the cardiovascular parameter module can be implemented locally on a user device.

The data quality module preferably functions to evaluate (e.g., determine, assess, etc.) a quality of the datasets (particularly but not exclusively the PG dataset and/or data associated with the PG dataset). Evaluating the quality can include detecting outliers or inliers within a dataset, determining (e.g., estimating, predicting) whether the system (e.g., sensors thereof) was used correctly, detecting motion (or other potential sources of artifacts or inaccuracies) in the data, detecting issues with the sensors (e.g., due to bias, broken or damaged sensors, etc.), and/or otherwise evaluating whether any degradation or inadequacies are present in the data. In a specific example, the data quality module can detect if a user moved during data collection and/or a body region placement of the user on a sensor (e.g., whether the body region covered the sensor, a contact pressure applied, etc.). However, the data quality module can detect any suitable aspects associated with the data quality.

The data quality module is preferably implemented on a user device or other local system, but alternatively can be partially or fully implemented on a remote system.

The data quality module can use one or more of: machine learning (e.g., deep learning, neural network, convolutional neural network, etc.), statistical analysis, regressions, decision trees, thresholding, classification, rules, heuristics, equations (e.g., weighted equations, etc.), selection (e.g., from a library), instance-based methods (e.g., nearest neighbor), regularization methods (e.g., ridge regression), Bayesian methods (e.g., Naïve Bayes, Markov), kernel methods, probability, deterministics, genetic programs, support vectors, and/or leverage any suitable algorithms or methods to assess the data quality. The data quality module can be trained using supervised learning, unsupervised learning, reinforcement learning, semi-supervised learning, and/or in any manner (e.g., via S500 methods).

Inputs to the data quality module can include: sensor data (e.g., images, motion data, etc.), auxiliary sensor data (e.g., images, lighting, audio data, temperature data, pressure data, etc.), information derived from sensor data (e.g., image attributes), historical information (e.g., historic image attributes from data collected from the same or different user during prior measurement sessions), user inputs, user parameters (e.g., user characteristics, height, weight, gender, skin tone, etc.), environmental parameters (e.g., weather, sunny, ambient lighting, situational information, auditory information, temperature information, etc.), sensor and/or user device make/model information (e.g., camera angle, solid angle of reception, type of light sensor, etc.), body region model (e.g., a light scattering model, etc.), light source (e.g., artificial light, natural light, direct light, indirect light, etc.), ambient light intensity, and/or any suitable information. In a first example, the inputs include one or more attributes (e.g., image attributes) extracted from sensor data. In a second example, the inputs include one or more features extracted from sensor data (e.g., features depicted in image, peaks, derivatives, etc.).

Inputs (particularly but not exclusively data) are preferably associated with a time window, but can include all historical data, predetermined historical data, current data, and/or any suitable data. The time window can depend on a target amount of data for determining the cardiovascular parameters (e.g., a threshold length of time), a processor capability, a memory limit, a sensor data rate, a number of data quality modules, and/or on any suitable information. The time window can be between 0.5 s-600 s or any range or value therebetween (e.g., 0.5, 1 s, 2 s, 4 s, 5 s, 8 s, 10 s, 12 s, 15 s, 20 s, 25 s, 50 s, 100 s, etc.), but can alternatively be less than 0.5 s or greater than 600 s. The time window can be a running time window (e.g., a time window can overlap another time window), sliding time window, discrete time windows (e.g., nonoverlapping time windows, nonconsecutive time windows, consecutive time windows, etc.), and/or any suitable time window. The dataset can be contiguous or noncontiguous. The dataset can optionally be a data segment (e.g., corresponding to a time window within a larger time range), wherein multiple data segments can optionally be aggregated (e.g., via S300 methods).

Outputs from the data quality module can include: a data quality, processed data (e.g., data processed to ensure that it achieves a target quality or metric), a flag (e.g., indicative of ‘good’ or ‘bad’ data), instructions to use (or possibly how to use or process) the data, instructions for how to improve the data collection, sensor use information (e.g., contact pressure, degree of coverage, orientation, etc.), a state of the user and/or system (e.g., a motion state, a use state, etc.), and/or any suitable outputs. The data quality can be a score, a classification, a probability (e.g., a probability of a given data quality, a probability of data being used to achieve a target or minimum accuracy or precision cardiovascular parameter, etc.), a quality, instructions, a flag, and/or any suitable output. The data quality can be binary (e.g., good vs bad, sufficient vs insufficient, yes vs no, useable vs unusable, acceptable vs unacceptable, etc.), a score, continuous (e.g., taking on any value such as between 0 to 1, 0 to ∞, 0 to 100, −∞ and ∞, etc.), discrete (e.g., taking on one of a discrete number of possible values, multiclass, etc.), and/or any suitable quality. The data quality can be a quality corresponding to input data and/or any other data. For example, when the input data includes image attributes extracted from sensor data (e.g., a video), the data quality can be a data quality for the input image attributes, for the sensor data, for PG data and/or any other image attributes extracted from the sensor data, and/or any other data. In some instances, the outputs from one or more data quality modules can be combined and/or processed to provide instructions, recommendations, guidance, and/or other information to the user (for example to improve or enhance a data quality for data to be collected).

The data quality can optionally be compared to one or more criteria (e.g., evaluating whether the data quality indicates high or low quality data, acceptable or unacceptable conditions, etc.). A criterion can be a threshold, a value (e.g., the data quality must equal a value), a presence/absence of a flag, and/or any other criterion. When the data quality meets one or more criteria: data can be stored, PG data can be generated (e.g., using images associated with the data quality), a cardiovascular parameter can be determined from PG data associated with the data quality, and/or any other action can be performed. When the data quality does not meet one or more criteria: the user can be guided (e.g., based on the data quality), data associated with the data quality can be rejected (e.g., erased, not stored, etc.), all or parts of the method can be reset and/or restart (e.g., acquiring new data), and/or any other action can be performed.

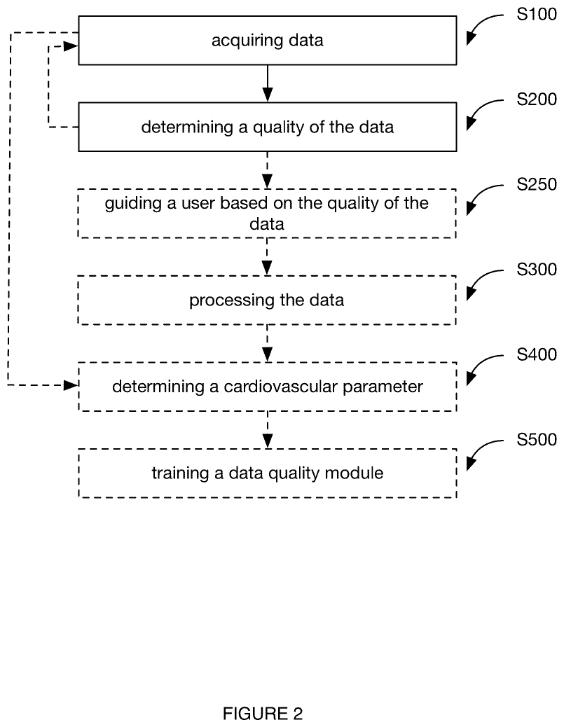

The system can include one or more data quality modules. When the system includes a plurality of data quality modules, the data quality modules can be correlated and/or uncorrelated from one another. Typically, each of the data quality modules uses different inputs, but one or more data quality modules can use the same inputs. Each of the data quality modules can provide the same or different outputs. Data quality modules, models included in a data quality module, and/or outputs thereof (e.g., the data quality, the classification of the PG dataset, etc.) can be combined (e.g., averaged, weighted average, using logical operators, using a set of rules, using voting, etc.), compared, selected from, voted on (e.g., using voting to select a most likely data quality; ranked voting, impartial voting, consensus voting, etc.), can be used separately, and/or can otherwise be used in tandem or isolation. In examples, logical operators used to combine one or more data quality modules and/or outputs therefrom can include: ‘AND’, ‘OR’, ‘XOR’, ‘NAND’, ‘NOR’, ‘XNOR’, ‘IF/THEN’, ‘IF/ELSE’, and/or any other operator. An example is shown in FIG. 3. For example, when one model classifies the PG dataset as having a low quality (e.g., ‘bad’, score less than a threshold, ‘poor’, ‘insufficient’, etc.), then the combined classification of the PG dataset can be low quality (e.g., even if the remaining the data quality modules indicate that the data quality is “good”). In a specific example, the logical operator between multiple data quality module outputs is an AND operator, wherein all data quality modules must output a ‘good’ score (e.g., indicating high quality data) in order for the data quality associated with input images (e.g., associated with PG data extracted from the input images) to be classified ‘good’. However, the data quality modules and/or outputs thereof can otherwise be combined.

Data quality modules can include a motion model, a body region contact model, a placement model, a signal quality model, and/or any other model. Models can be specific to: a user device make and/or model, a sensor (e.g., camera or other image senor, motion sensor, etc.) make and/or model, a specific sensor instance, an environmental parameter, a user parameter, and/or any other parameter.

The motion model can function to determine a motion parameter for the user and/or user device. The motion parameter preferably indicates whether the user and/or the user device is moving (e.g., motion exceeds a threshold speed, motion exceeds a threshold acceleration, motion exceeds a threshold distance, etc.) and/or was moving within a threshold time period. Additionally or alternatively, the motion parameter can indicate whether the user pose and/or user device pose is within a threshold pose range. However, the motion parameter can indicate any metric (e.g., any data quality metric). One or more thresholds defining acceptable and/or unacceptable motion (e.g., wherein acceptable motion corresponds to high quality data and/or wherein unacceptable motion corresponds to low quality data) can optionally be defined (e.g., empirically defined) during model training (e.g., S500), but can additionally or alternatively be predetermined, be otherwise determined, and/or not be used for the motion model.

The motion model can include a classifier, set of thresholds for each input, heuristic, machine learning model (e.g., NN, CNN, DNN, etc.), statistical analysis, regressions, decision trees, rules, equations, selection, instance-based methods, regularization methods, Bayesian methods, kernel methods, probability, deterministics, genetic programs, support vectors, and/or any other model. The motion model is preferably a single model outputting a motion parameter (e.g., a binary classification), but can alternatively be multiple models wherein the motion parameter output is determined from multiple model outputs.

The motion model can receive as inputs: accelerometer data (e.g., in one or more of x/y/z coordinates), gyroscope data (e.g., in one or more of x/y/z coordinates), gravity vector data (e.g., in one or more of x/y/z coordinates), location information, environmental data, and/or any other suitable data (e.g., any other data quality module input data). In an example, the motion model input includes gravity (e.g., xyz vector), acceleration (e.g., xyz vector), rotation (e.g., xyz vector), and attitude (e.g., vector including pitch, yaw, and roll). In a specific example, the motion model input includes only gravity, acceleration, rotation, and attitude. The input data is preferably concurrently sampled with the measurements used for other data quality modules and/or cardiovascular parameter modules, but can alternatively be contemporaneously sampled, asynchronously sampled, and/or otherwise sampled relative to other modules.

The motion model can output the motion parameter, wherein the motion parameter can be a classification (e.g., binary, multiclass, etc.), a score, continuous, discrete, and/or be any other parameter type. In examples, the motion parameter can be associated with: user and/or user device motion, user and/or user device pose (e.g., position and/or orientation), a data quality (e.g., a data quality classification for the input data and/or for a PG dataset associated with the input data), a combination thereof, and/or any other parameter.

In specific examples, the motion model can output a classification of a user or user device motion (e.g., a yes/no classification for whether the user is moving, a yes/no classification for whether the user has moved recently, a good/bad classification for whether the user device is experience acceptable/unacceptable motion, etc.), a value for the user or user device motion, a classification of user and/or user device pose, a classification of a PG dataset (e.g., a PG dataset that was acquired concurrently or contemporaneously with the input data, a PG dataset derived from the input data, etc.), guidance for adjusting (e.g., improving) user and/or user device motion, and/or any suitable output. In a first illustrative example, the motion model can output a binary classification corresponding to ‘acceptable motion’ (e.g., ‘correct motion’) and ‘unacceptable motion’ (e.g., ‘incorrect motion’). In a second illustrative example, the motion model can output a multiclass classification corresponding to specific acceptable and/or unacceptable conditions (e.g., the acceptable and/or unacceptable conditions in S500).

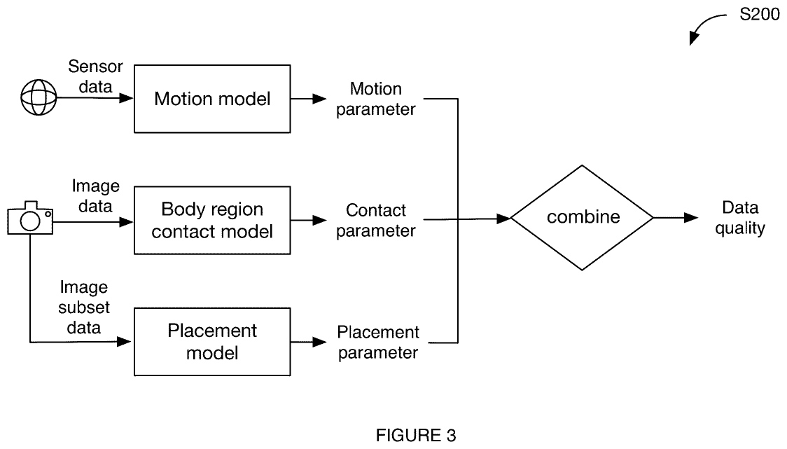

An example is shown in FIG. 4.

However, the motion model can be otherwise configured.

The body region contact model (e.g., a body region detection model) can function to determine a contact parameter for a body region (e.g., a finger) relative to a sensor (e.g., image sensor). The contact parameter preferably indicates whether a body region is in contact with the sensor. Additionally or alternatively, the contact parameter can indicate whether the body region is within a FOV of a sensor (e.g., within a threshold extent of FOV coverage), whether the body region is in contact with the sensor within a threshold extent of contact coverage, whether the body region is in contact with the sensor within a threshold pressure range, and/or whether the body region pose is within a threshold pose range relative to the sensor. However, the contact parameter can indicate any metric (e.g., any data quality metric). One or more thresholds defining acceptable and/or unacceptable body region contact (e.g., wherein acceptable body region contact corresponds to high quality data and/or wherein unacceptable body region contact corresponds to low quality data) can optionally be defined (e.g., empirically defined) during model training (e.g., S500), but can additionally or alternatively be predetermined, be otherwise determined, and/or not be used for the body region contact model.

The body region contact model can include a classifier, set of thresholds for each input, heuristic, machine learning model (e.g., NN, CNN, DNN, etc.), statistical analysis, regressions, decision trees, rules, equations, selection, instance-based methods, regularization methods, Bayesian methods, kernel methods, probability, deterministics, genetic programs, support vectors, and/or any other model.

The body region contact model is preferably a single model outputting a contact parameter (e.g., a binary classification), but can alternatively be multiple models wherein the contact parameter output is determined from multiple model outputs. In a first specific example, one model functions to detect body region contact presence and/or an extent of contact coverage. In a second specific example, one model functions to detect body region contact presence, an extent of contact coverage, a body region pose, and/or a contact pressure. In a third specific example, the body region contact model includes two models, wherein a first model functions to detect body region contact presence and/or an extent of contact coverage, and a second model functions to detect contact pressure and/or body region pose.

The body region contact model can receive as inputs: image attributes, images, depth datasets, other sensor data, and/or any other suitable data (e.g., any other data quality module input data). For example, the body region contact model input can include total luminance, total chroma (e.g., total red, total blue, and/or total green chroma values; only total red and total blue chroma values; etc.), and/or any other image attribute for one or more images. The image attributes can be optionally aggregated across a set of images (e.g., an array of one or more image attribute values for each image; a single value for each image attribute corresponding to the entire set of images; etc.). In an illustrative example, the body region contact model input includes total luminance, total red chroma, and total blue chroma values for each frame of a video. In a specific example, an image sensor can sample a 2 s video at 60 FPS (120 frames), wherein a total luminance, total red chroma, and total blue chroma is determined for each frame (e.g., the input data includes three arrays with dimensions [120×1]). The input data is preferably concurrently sampled with the measurements used for other data quality modules and/or cardiovascular parameter modules, but can alternatively be contemporaneously sampled, asynchronously sampled, and/or otherwise sampled relative to other modules.

The body region contact model can output the contact parameter and optionally a confidence score for the contact parameter, wherein the contact parameter can be a classification (e.g., binary, multiclass, etc.), a score, continuous, discrete, and/or be any other parameter type. In examples, the contact parameter can be associated with: body region contact with the sensor (e.g., contact pressure, contact presence, extent of contact coverage, etc.), body region detection in a sensor FOV (e.g., body region presence, extent of FOV coverage), body region pose relative to the sensor for the body region (e.g., position and/or orientation; only the body region position; etc.), a data quality (e.g., a data quality classification for the input data and/or for a PG dataset associated with the input data), a combination thereof, and/or any other parameter.

In specific examples, the body region contact model can output a classification of a user body region coverage of the sensor (e.g., a presence/absence of the body region within a FOV of an image sensor; presence/absence of body region contact with the sensor; a yes/no classification for whether the body region contact coverage and/or FOV coverage is above a threshold value, etc.), a value for the extent of contact coverage, a classification of a contact pressure (e.g., good/bad or acceptable/unacceptable contact pressure), a value for the contact pressure, a classification of a PG dataset (e.g., a PG dataset that was acquired concurrently or contemporaneously with the input data, a PG dataset derived from the input data, etc.), guidance for adjusting (e.g., improving) body region contact, and/or any suitable output can be generated. In a first illustrative example, the body region contact model can output a binary classification corresponding to ‘body region detected’ and ‘body region not detected’. In a second illustrative example, the body region contact model can output a multiclass classification corresponding to specific acceptable and/or unacceptable conditions (e.g., the acceptable and/or unacceptable conditions in S500).

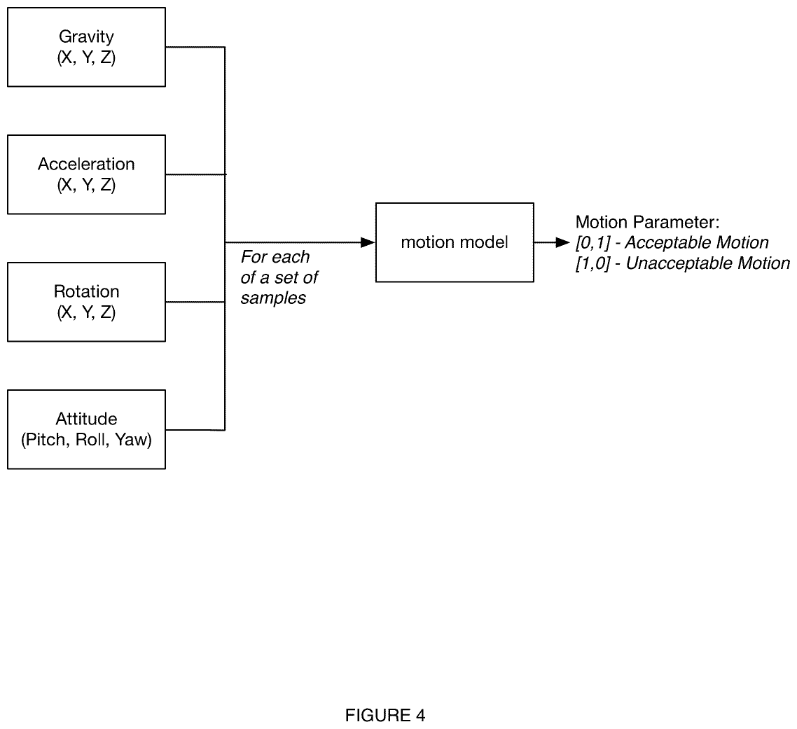

An example is shown in FIG. 5.

However, the body region contact model can be otherwise configured.

The placement model can function to determine a placement parameter (e.g., a pose parameter, a pressure parameter, a contact parameter, etc.) for a body region (e.g., finger) relative to a sensor (e.g., image sensor). The placement parameter preferably indicates which portion of the image sensor FOV is covered by the body region. Additionally or alternatively, the placement parameter can indicate whether the body region is in contact with the sensor within a threshold pressure range, whether the body region placement is within a threshold pose range relative to the sensor (e.g., a threshold distance and/or a threshold orientation relative to the image sensor), whether a body region is within a FOV of a sensor (e.g., within a threshold extent of FOV coverage), and/or whether a body region is in contact with the sensor (e.g., within a threshold extent of contact coverage). However, the placement parameter can indicate any metric (e.g., any data quality metric). One or more thresholds defining acceptable and/or unacceptable body region placement (e.g., wherein acceptable placement corresponds to high quality data and/or wherein unacceptable placement corresponds to low quality data) can optionally be defined (e.g., empirically defined) during model training (e.g., S500), but can additionally or alternatively be predetermined, be otherwise determined, and/or not be used for the placement model.

The placement model can include a classifier, set of thresholds for each input, heuristic, machine learning model (e.g., NN, CNN, DNN, etc.), statistical analysis, regressions, decision trees, rules, equations, selection, instance-based methods, regularization methods, Bayesian methods, kernel methods, probability, deterministics, genetic programs, support vectors, and/or any other model. The placement model is preferably a single model outputting a placement parameter (e.g., a binary classification), but can alternatively be multiple models wherein the placement parameter output is determined from multiple model outputs.

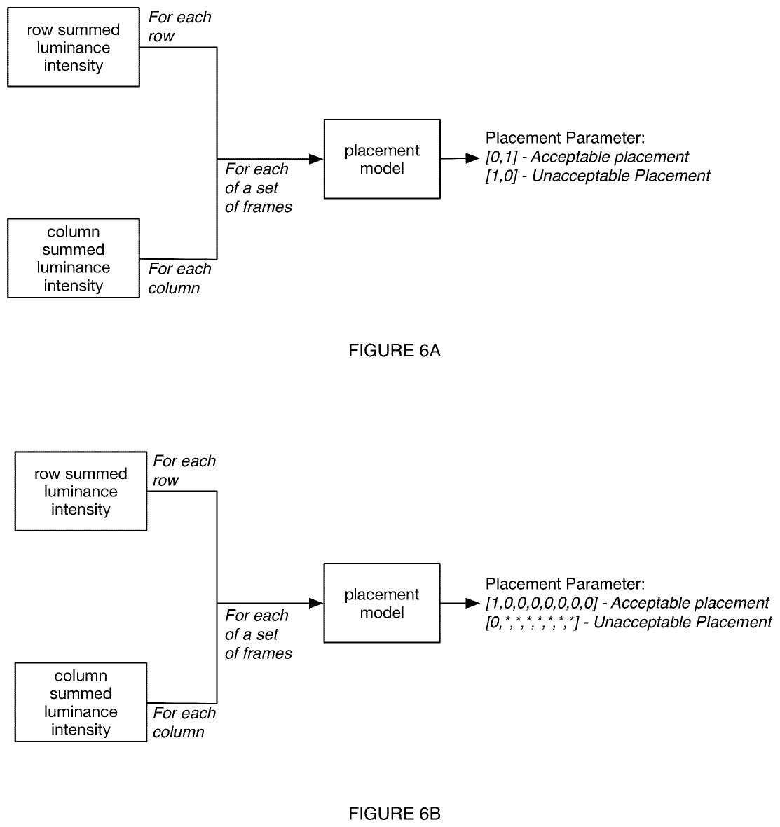

The placement model can receive the same or different inputs as the body region contact model. In examples, the placement model can receive as inputs: image attributes, images, depth datasets, other sensor data, and/or any other suitable data (e.g., any other data quality module input data). For example, the placement model input can include summed luminance across a subset of pixels in an image, summed chroma (e.g., summed red, summed blue, and/or summed green chroma values) across a subset of pixels in an image, and/or any other image attribute for one or more images. The subset of pixels can be distinct image subregions and/or overlapping subregions. In an illustrative example, the placement model input includes an array of summed luminance for each pixel row and/or column of an image (e.g., each row and/or column of the entire image or a portion of the image). The image attributes can be optionally aggregated across a set of images (e.g., an array of one or more image attribute values for each image; a single value for each image attribute corresponding to the entire set of images; etc.). An example is shown in FIG. 7. In a specific example, an image sensor can sample a 2 s video at 120 FPS (120 frames), wherein each frame has a resolution of 1280×720 pixels; a summed luminance is determined for each row (e.g., the input data across the frames includes an array with dimensions [120×1280]) and column (e.g., the input data across the frames includes an array with dimensions [120×720]). The input data is preferably concurrently sampled with the measurements used for other data quality modules and/or cardiovascular parameter modules, but can alternatively be contemporaneously sampled, asynchronously sampled, and/or otherwise sampled relative to other modules.

The placement model can return the same or different outputs as the body region contact model. The placement model can output the placement parameter, wherein the placement parameter can be a classification (e.g., binary, multiclass, etc.), a score, continuous, discrete, and/or be any other parameter type. In examples, the placement parameter can be associated with: body region pose relative to the sensor for the body region (e.g., position and/or orientation; only the body region position; etc.), body region contact with the sensor (e.g., contact pressure, contact presence, extent of contact coverage, etc.), a data quality (e.g., a data quality classification for the input data and/or for a PG dataset associated with the input data), a combination thereof, and/or any other parameter.

In specific examples, the placement model can output a pose of the body region relative to the sensor (e.g., position and/or orientation), a classification of a pose of the body region relative to the sensor (e.g., a yes/no classification for whether the body region pose is placed within a threshold pose range, acceptable/unacceptable pose, a multiclass classification indicating the pose, etc.), a position of the body region relative to the sensor (e.g., a distance from the sensor center), a classification of a position of the body region relative to the sensor (e.g., a yes/no classification for whether the body region is placed within a threshold distance to the sensor center, acceptable/unacceptable position, a multiclass classification indicating the pose, etc.), a classification of a contact pressure (e.g., good/bad or acceptable/unacceptable contact pressure), a value for the contact pressure, classification of a body region coverage of the sensor (e.g., a yes/no classification for whether the body region contact coverage and/or FOV coverage is above a threshold value, etc.), a value for the extent of contact coverage, guidance for adjusting (e.g., improving) placement (e.g., including pose and/or contact pressure) of the body region, instructions for how to adjust (e.g., improve) a PG dataset quality (e.g., via body region pose guidance), a classification of a PG dataset (e.g., a PG dataset that was acquired concurrently or contemporaneously with the input data, a PG dataset derived from the input data, etc.), a confidence score (e.g., for one or more classifications), and/or any suitable output can be generated.

In a first illustrative example, the placement model can output a binary classification corresponding to ‘acceptable body region placement’ and ‘unacceptable body region placement’. In a second illustrative example, the placement model can output a multiclass classification corresponding to specific acceptable and/or unacceptable conditions (e.g., the acceptable and/or unacceptable conditions in S500). For example, the multiclass classification can include: ‘acceptable body region placement’, ‘contact pressure too high’, ‘contact pressure too low’, ‘body region too far down’, ‘body region too far up’, ‘body region too far left’, ‘body region too far right’, ‘body region motion too high’, and/or any other classification.

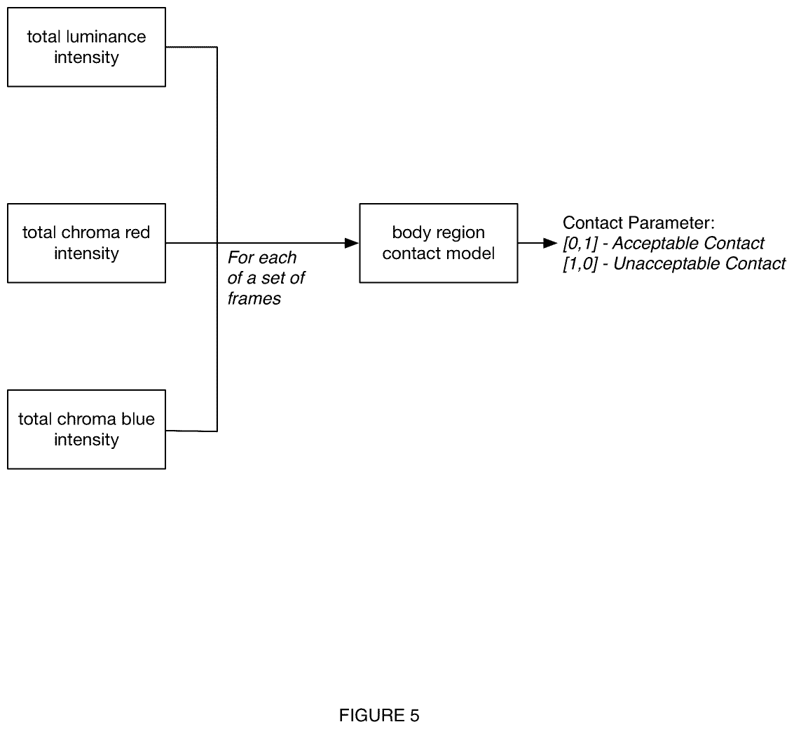

Examples are shown in FIG. 6A, FIG. 6B, and FIG. 6C.

However, the placement model can be otherwise configured.

The signal quality model can function to determine a signal quality parameter for the PG dataset (e.g., after the PG dataset has been classified as ‘high quality’ on the user device based on the motion model, body region contact model, and/or placement model). The signal quality parameter preferably indicates whether the PG signal quality is low (e.g., due to the body region being cold), but can alternatively indicate any other metric (e.g., any data quality metric).

The signal quality model is preferably located on a remote computing system, but can alternatively be located on a local computing system and/or distributed between local and remote computing systems.

The signal quality model can take as input all or portion of PG dataset (e.g., received from the user device), any sensor data, and/or any other suitable data (e.g., any other data quality module input data). The signal quality model can output one or more signal quality parameters, wherein the signal quality parameter can be a classification (e.g., binary, multiclass, etc.), a score, continuous, discrete, and/or be any other parameter type. In examples, the signal quality parameter can be associated with: body region temperature, a data quality (e.g., a data quality classification for the PG dataset), a combination thereof, and/or any other parameter. The signal quality parameter can be determined based on a processed or unprocessed PG dataset (e.g., the raw PG dataset, one or more segments of the PG dataset, a derivative of all or a portion of the PG dataset, a second derivative of all or a portion of the PG dataset, a third derivative of all or a portion of the PG dataset, etc.). In examples, the signal quality parameter can include or be based on: a signal power metric, a correlation metric (e.g., local correlation metric and/or a global correlation metric), a fit metric (e.g., based on a fiducial model fit to the PG dataset), statistical analyses of the PG dataset (e.g., outlier detection), and/or any other metrics. In a specific example, the signal quality model can output a binary classification indicating whether all or a portion of the PG dataset (e.g., at least a threshold number of PG dataset segments) satisfies one or more signal quality criteria (e.g., a signal power criterion, a correlation criterion, a fit criterion, etc.). In specific examples, a signal quality criterion can evaluate whether a signal quality parameter is greater than a threshold, less than a threshold, passes an outlier filter, passes a statistical analysis filter, and/or any other evaluation.

However, the signal quality model can be otherwise configured.

In specific examples, two models can be used (e.g., a body region contact model and a placement model, a motion model and a placement model, a motion model and a body region contact model, etc.), three models can be used (e.g., a body region contact model, a placement model, and a motion model; a motion model and two body region contact models, etc.), more than three models can be used (e.g., duplicate models, additional models such as models that process chroma or color channels separately, etc.), and/or any suitable models can be used. Additionally or alternatively, a single model can be trained that processes the inputs and/or generates the outputs of two or more of the separated models. However, any suitable models can be used.

However, the data quality module can be otherwise configured.

A cardiovascular parameter module preferably functions to determine the cardiovascular parameter. The cardiovascular parameter module can additionally or alternatively function to determine or process (e.g., segment, denoise, etc.) a PG dataset (e.g., from a set of images, as disclosed in U.S. patent application Ser. No. 17/866,185 titled ‘METHOD AND SYSTEM FOR CARDIOVASCULAR DISEASE ASSESSMENT AND MANAGEMENT’ filed on 15 Jul. 2022 which is incorporated in its entirety by this reference, etc.), and/or can otherwise function.

The cardiovascular parameter module can be local, remote, distributed, or otherwise arranged relative to any other system or module. In a first example, one or more inputs are determined locally (e.g., via a user device) and transmitted to a cardiovascular parameter module implemented on a remote computing system. In this example, one or more outputs from the cardiovascular parameter module can optionally be transmitted back to a local system (e.g., the user device). In a second example, the cardiovascular module is implemented locally on a user device or other local system.

The output of the cardiovascular parameter module can be one or more cardiovascular parameters, a processed dataset (e.g., processed PG dataset), and/or any other suitable output. The cardiovascular parameter module can receive as inputs: image attributes for one or more images (e.g., PG data), image features, images, environmental parameters, other sensor data, and/or any other suitable data (e.g., any other data quality module input data). Image features are preferably different from image attributes, but can alternatively be the same as image attributes. All or parts of the input data is preferably the same data and/or extracted from the same data used by one or more data quality modules, but can alternatively not be the same data used by one or more data quality modules. For example, the cardiovascular parameter module input(s) can be derived from all or a subset of a series of images, wherein the same series of images was used to determine inputs for one or more data quality modules. In a specific example, a first set of image features and/or attributes can be extracted from a series of images to be used as input into one or more data quality modules; a second set of image features and/or attributes (e.g., PG data) can be extracted from all or a subset of the series of images (e.g., wherein the subset is determined based on the data quality module output) can be used as input in the cardiovascular parameter module.

The cardiovascular parameter(s) are preferably determined from data (e.g., PG data) that is associated with a high data quality (e.g., as determined by the data quality module(s)), but can be determined using data with a low data quality, and/or any suitable data. In a first variant, an entire sensor data sample is validated by the data quality module (e.g., validated as high data quality), wherein the validated sensor data sample and/or data extracted therefrom (e.g., image attributes and/or image features) can be used as an input into the cardiovascular parameter model. In a second variant, a portion of a sensor data is validated by the data quality module (e.g., a subset of frames in a video, a subset of pixels in one or more frames, etc.). For example, the output of the data quality modules is used to select high quality images, wherein image features and/or image attributes extracted from the high data quality images are used as inputs into the cardiovascular parameter module. In a third variant, the cardiovascular parameter input can be different from the data validated by the data quality module.

The cardiovascular parameters are preferably determined using a time series of PG data (e.g., a times series of multiple high quality PG datasets), but can be determined using any suitable data. For example, a cardiovascular parameter can be determined using PG datasets (or other datasets) that include at least a threshold number of seconds of data. The threshold number of seconds can be between 4 s-600 s or any range or value therebetween (e.g., 5 s, 10 s, 15 s, 20 s, 30 s, 45 s, 60 s, 120 s, 300 s, 600 s, etc.), but can alternatively be less than 4 s or greater than 600 s. The time series of data can be contiguous (e.g., PG data extracted from an uninterrupted segment of a video) or noncontiguous (e.g., PG data extracted from discrete, non-neighboring segments of a video). The time series of data can optionally be accumulated segments of an initial timeseries of data (e.g., accumulated via S300 methods). The segments can correspond to a predetermined length of time, a predetermined data size, a variable length of time, a variable data size, and/or any other parameter. In a first example, segment length is predetermined. The segment length can be between 0.2 s-60 s or any range or value therebetween (e.g., 0.5 s-5 s, 1 s-3 s, 1 s, 2 s, 3 s, etc.), but can alternatively be less than 0.2 s or greater than 60 s. In a second example, segment length is determined based on one or more data quality module outputs (e.g., the segment corresponds to a segment of high data quality; a segment ends when data quality crosses a threshold from ‘good’ to ‘bad’; etc.).

The cardiovascular parameter can be determined using a transformation, using an equation, using a machine learning algorithm, using a particle filter, any method in S400, and/or in any suitable manner.

However, the cardiovascular parameter module can be otherwise configured.

The storage module preferably functions to store the datasets and/or cardiovascular parameters. The storage module can store the datasets and/or cardiovascular parameters can locally and/or remotely. The storage modules can correspond to long-term (e.g., permanent) memory or short-term (e.g., transient) memory. Examples of storage modules include caches, buffers (e.g., image buffers), databases, look-up tables, RAM, ROM, and/or any type of memory. However, the storage module can be otherwise configured.

However, the computing system can be otherwise configured.

### 5. METHOD

As shown in FIG. 2, the method can include acquiring data S100 and determining a quality of the data S200. The method can optionally include guiding a user based on the quality of the data S250, processing the data S300, determining a cardiovascular parameter S400, training a data quality module S500, and/or any suitable steps. All or portions of the method can be performed by one or more components of the system, by a user, and/or by any other suitable system.

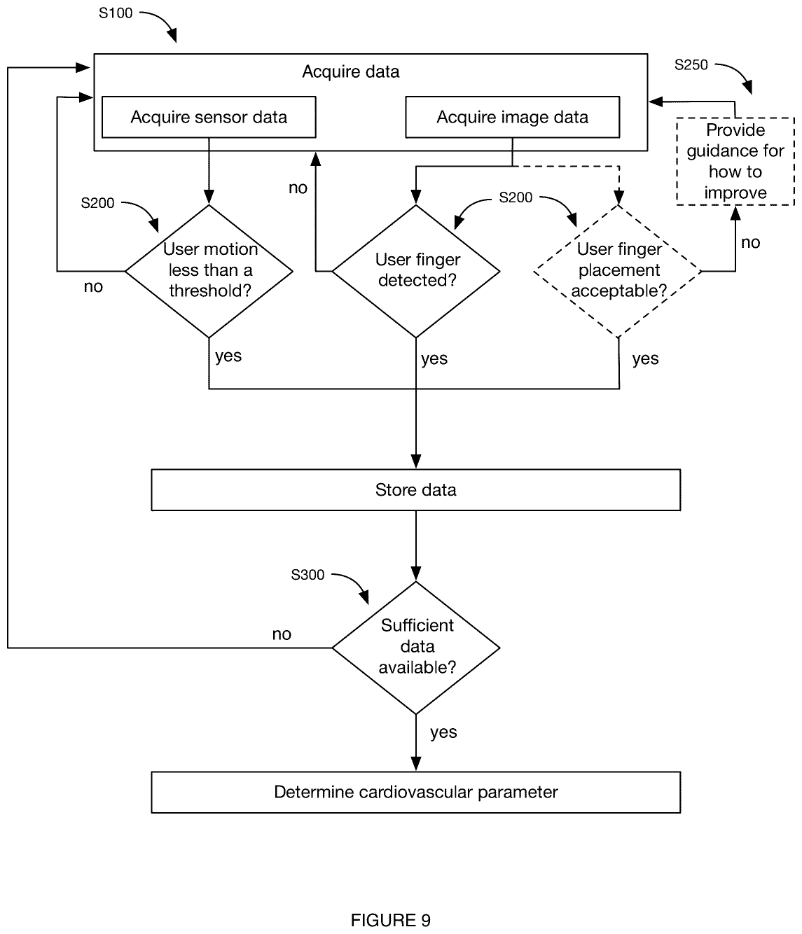

All or portions of the method can be performed automatically (e.g., in response to one or more criteria being met), manually, semi-automatically, and/or otherwise performed. All or portions of the method can be performed after calibration (e.g., with a blood pressure cuff, ECG system, and/or any other calibration system), during calibration, without calibration, and/or at any other time. An example of the method including calibration is shown in FIG. 11. All or portions of the method can be performed in real-time (e.g., data can be processed contemporaneously with and or concurrently with data acquisition), offline (e.g., with a delay or lag between data acquisition and data processing), iteratively, asynchronously, periodically, and/or with any suitable timing. In an example, the method can include acquiring data segments (e.g., video segments), wherein a data quality is determined in real-time for each segment (e.g., substantially immediately after the segment is acquired), and wherein a high quality PG dataset is generated contemporaneously with acquiring the data segments and/or contemporaneously with determining the data quality for the data segments (e.g., accumulating data segments to form the high quality PG dataset as each segment is validated). Different data segments can overlap (e.g., share data, be from overlapping timestamps) or be distinct.

Acquiring data S100 functions to acquire one or more datasets that can be used to determine a dataset quality (e.g., in S200), determine cardiovascular parameters (e.g., in S400), and/or can otherwise be used. S100 can be performed in response to a request, after (e.g., in response to) a user placing a body region on a sensor, after or during calibration, and/or at any other time. S100 is preferably performed using one or more sensors (e.g., to acquire the data), but can be performed by a computing system (e.g., to retrieve one or more datasets from a storage module) and/or by any suitable component.

S100 can include acquiring motion datasets (e.g., datasets associated with and/or that can be used to determine a motion state of a user and/or user device), image datasets, information extracted from image datasets (e.g., image attributes, image features, etc.), PG datasets (e.g., datasets associated with an arterial pressure), environmental datasets (e.g., datasets associated with an environmental property such as ambient light), and/or acquiring any suitable datasets. The PG datasets preferably include and/or are derived from an image set of a body region of a user (e.g., PG datasets can be features or attributes extracted from an image set acquired with a body region of the user in contact with the image sensor and/or optics thereof), but can additionally or alternatively include or be derived from a blood pressure sensor (e.g., blood pressure cuff, sphygmomanometer, etc.), plethysmogram sensor, and/or any suitable data source.

When more than one dataset is acquired, the datasets are preferably acquired contemporaneously and/or simultaneously (e.g., concurrently). However, the datasets can be acquired asynchronously, offline, delayed, and/or with any suitable timing.

Each dataset is preferably continuously acquired (e.g., for the duration of the method, until sufficient data is collected, until a trigger indicating that data acquisition can end, until a data quality changes, until a data quality changes by a threshold amount, until a user ends the data acquisition, until an API or application performing or hosting the method indicates an ending, until a user removes the body region from the sensor, etc.), but can be acquired intermittently, at predetermined times or frequency, at discrete times, and/or with any suitable timing.

Each dataset preferably corresponds to a time window that is at least a threshold number of seconds, but can alternatively be associated with any number of seconds and/or not be associated with a time window. The threshold number of seconds can be between is-600 s or any range or value therebetween (e.g., 2 s, 4 s, 5 s, 8 s, 10 s, 12 s, 15 s, 20 s, 25 s, 50 s, loos, etc.), but can alternatively be less than is or greater than 600 s. The time window can be a running time window, sliding time window, discrete time windows, and/or any suitable time window. The dataset can be contiguous or noncontiguous. The dataset can optionally be a data segment corresponding to the time window (e.g., within a larger time range), wherein multiple data segments can optionally be aggregated (e.g., via S300 methods).

S100 can include processing the datasets. For example, processing the datasets can be performed in and/or include the same or different steps as processing the datasets as discussed below in S300. However, the datasets can be processed in any manner.

S100 can include storing the dataset(s) (e.g., using the storage module). The dataset(s) can be stored indefinitely, for a predetermined amount of time, until a condition is met (e.g., until a data quality has been evaluated, until a cardiovascular parameter has been calculated, until a threshold amount of data with a target quality has been acquired, until attributes or features have been extracted, etc.). Datasets can be stored based on their quality, based on the data type, based on data completeness, and/or based on any suitable criteria. For example, only datasets with a high quality (e.g., meeting a criterion such as a good classification) can be stored. In an illustrative example of storing a dataset, an image buffer is generated while the image sensor is acquiring a video, wherein memory is temporarily allocated for each video frame (e.g., including relevant metadata, wherein metadata can include timestamps, resolutions, etc.). The video frames can then be provided to the data quality module for processing and/or analysis, wherein the image buffer is released back to the image sensor once each video frame's image buffer has been processed by the data quality module (e.g., transformed into luma and chroma values, image features extracted, etc.). However, all datasets can be stored and/or any suitable datasets can be stored based on any suitable criteria.

Determining a quality of the data S200 preferably functions to determine (e.g., assess, evaluate, etc.) a quality of dataset (e.g., acquired in S100). The quality is preferably used to determine whether a dataset can be used to determine a cardiovascular parameter (e.g., in S400, to achieve a target accuracy, to achieve a minimum accuracy, to achieve a target precision, to achieve a minimum precision, etc.), but can additionally or alternatively be used to determine whether to stop or continue data acquisition, and/or can otherwise be used. The quality is preferably a binary classification (e.g., ‘good’ vs ‘bad’, ‘acceptable’ vs ‘unacceptable’, etc.), but can be a continuous value, a nonbinary classification, and/or have any suitable format. S200 can be performed by a data quality module (e.g., of a local or remote computing system) and/or by any suitable component.

S200 is preferably performed on data acquired in S100, but can be performed on any suitable data. S200 is preferably performed on data segments corresponding to time windows, but can be performed on any suitable data. The time windows are preferably smaller than the time windows used to determine the cardiovascular parameter (e.g., in S400) and/or used to process the data (e.g., in S300), but can be the same size as and/or longer than the processed data windows. For example, the length of the data quality time windows can be between 0.5 s-600 s or any range or value therebetween (e.g., 0.5 s, 1 s, 2 s, 4 s, 5 s, 8 s, 10 s, 12 s, 15 s, 20 s, 25 s, 50 s, loos, etc.), but can alternatively be less than 0.5 s or greater than 600 s. The time window can be a running time window, sliding time window, discrete time windows, and/or any suitable time window. The time window (e.g., and the corresponding number of frames in the corresponding data segment) is preferably predetermined, but can alternatively be empirically determined (e.g., how long a human can remain still) and/or otherwise determined (e.g., using ablation analysis to determine the minimum number of frames to accurately determine data quality).

S200 can be performed in parallel or series for different time windows. In an illustrative example, when 10 s of data are desirable for processing or determining a cardiovascular parameter, five (or more) instances of S200 can be performed simultaneously on 2 s segments of the data. In a second illustrative example, as data is acquired (e.g., as new time windows are populated with data), a data quality can be evaluated (e.g., for each 2 s segment of data). In a third illustrative example, a subsequent S200 iteration can be performed (e.g., on a new time window) after a prior S200 iteration (e.g., on a previous time window) failed to produce acceptable quality data. However, S200 can be performed for any suitable time windows and/or with any suitable timing.

S200 can be performed using one or more models (e.g., models in the data quality module). The models can use one or more of: machine learning (e.g., deep learning, neural network, convolutional neural network, etc.), statistical analysis, regressions, decision trees, thresholding, classification, rules, heuristics, equations (e.g., weighted equations, etc.), selection (e.g., from a library), instance-based methods (e.g., nearest neighbor), regularization methods (e.g., ridge regression), Bayesian methods (e.g., Naïve Bayes, Markov), kernel methods, probability, deterministics, genetic programs, support vectors, and/or leverage any suitable algorithms or methods to assess the data quality.

In a specific example, S200 can be performed using a motion model, a body region contact model, and/or a placement model. When a plurality of models is used, each model can be associated with an aspect of the data quality, a data type, an amount of data (e.g., time window duration, sensor reading frequency, etc.), a data quality (e.g., a first model can be used to determine whether data achieves a first quality and a second model can be used to determine whether data achieves a second quality, where the first and second model can use the same or different inputs), and/or can be associated with any suitable data or information.

In a first variant, S200 includes using a motion model to output a data quality. Data acquired via S100 (e.g., raw, aggregated, processed, features extracted from the data, attributes extracted from the data, etc.) can be inputted to the motion model, wherein the motion model outputs a classification. For example, the data can be user device motion sensor data (e.g., gyroscope, accelerometer, and/or gravity vector data). In a first embodiment, the classification can be based on a set of thresholds (e.g., an acceptable motion classification when all thresholds or other conditions are met, an unacceptable motion classification when one or more thresholds or other conditions are not met). In a second embodiment, the classification can be determined (e.g., predicted) by a model trained to predict an acceptable/unacceptable classification based on training data (e.g., sensor data labeled with acceptable/unacceptable classifications).

In a second variant, S200 includes using a body region contact model to output a data quality. Data acquired via S100 (e.g., raw, aggregated, processed, features extracted from the data, attributes extracted from the data, etc.) can be inputted to the body region contact model, wherein the body region contact model outputs a classification. For example, the data acquired via S100 can be a set of images (e.g., a data sample corresponding to a segment of a video), wherein image attributes can be extracted from the set of images and used as inputs for body region contact model. In examples, the image attributes can include total chroma for one or more channels (e.g., total chroma for each of red, blue, and green channels; total chroma for only red and blue channels, etc.), total luminance, and/or any other image attribute. The image attributes can be optionally aggregated across the set of images (e.g., an array of one or more image attribute values for each image; a single value for each image attribute corresponding to the entire set of images; etc.). In a first embodiment, the data quality output (e.g., a classification) is based on set of thresholds (e.g., predetermined thresholds corresponding to acceptable body region contact conditions). In a second embodiment, the data quality output is determined (e.g., predicted) by a body region contact model trained to predict ‘body region detected’ (e.g., associated with one or more acceptable body region contact conditions) or ‘body region not detected’ (e.g., associated with one or more unacceptable body region contact conditions) based on training data including image sets and/or aggregated image attributes labeled with ‘body region detected’ or ‘body region not detected’ (e.g., via S500 methods).

In a third variant, S200 includes using a placement model to output a data quality. Data acquired via S100 (e.g., raw, aggregated, processed, features extracted from the data, attributes extracted from the data, etc.) can be inputted to the placement model, wherein the placement model outputs a classification. For example, the data acquired via S100 can be a set of images, wherein image attributes can be extracted from the set of images and used as inputs for placement model. The set of images can the same set of images or a different set of images as used for the body region contact model. In an example, the image attributes can include luminance (and/or any other channel) summed across one or more image subregions (e.g., aggregate luminance for each row, aggregate luminance for each column, etc.). The image attributes can be optionally aggregated across the set of images (e.g., an array of one or more image attribute values for each image; a single value for each image attribute corresponding to the entire set of images; etc.). In a first embodiment, the data quality output (e.g., a classification) is based on set of thresholds (e.g., predetermined thresholds corresponding to acceptable body region placement conditions). For example, each image subregion can optionally have a different threshold. In a second embodiment, the data quality output is determined (e.g., predicted) by a placement model trained to predict ‘acceptable body region placement’ (e.g., associated with one or more acceptable body region placement conditions) or ‘unacceptable body region placement’ (e.g., associated with one or more unacceptable body region placement conditions) based on training data including image sets and/or aggregated image attributes labeled with ‘acceptable body region placement’ or ‘unacceptable body region placement’ (e.g., via S500 methods). Alternatively, the data quality output is determined by a placement model trained to predict a guidance label (e.g., ‘acceptable finger placement’, ‘finger pressure too high’, ‘finger pressure too low’, ‘finger too far down’, ‘finger too far up’, ‘finger too far left’, ‘finger too far right’, ‘finger motion too high’, etc.) based on training data labeled with the guidance labels.

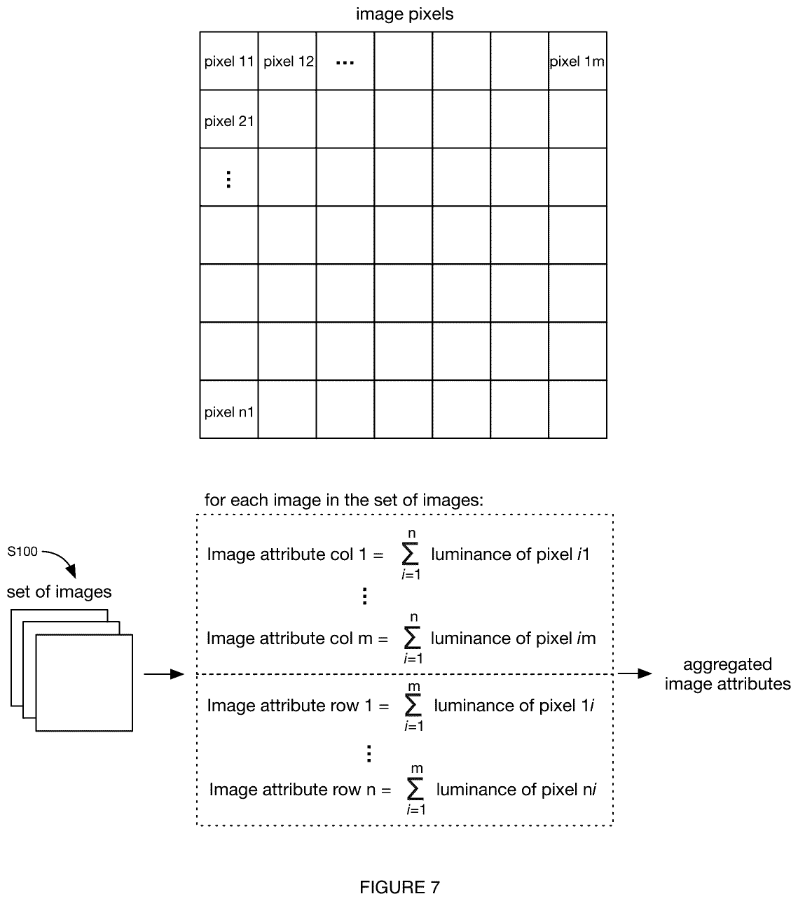

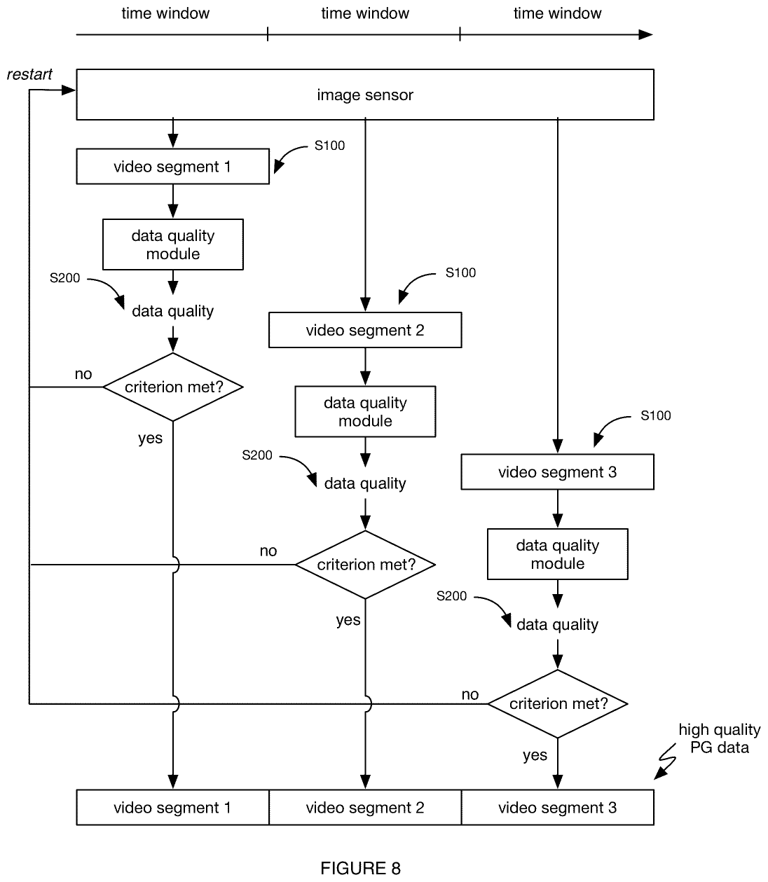

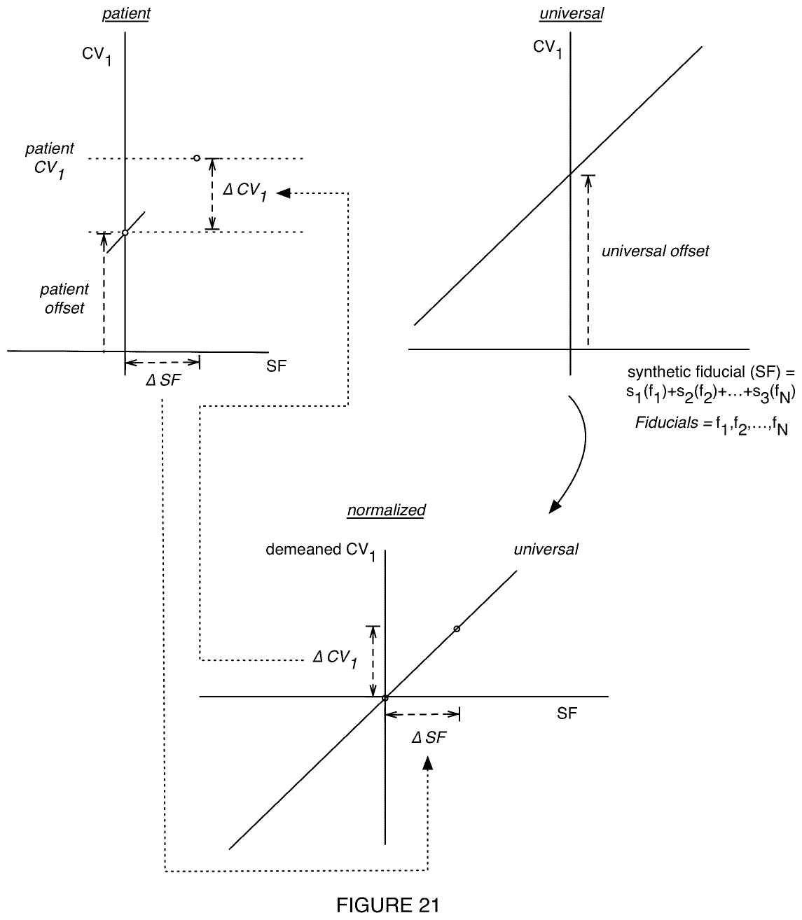

When a plurality of models is used, the data quality can be determined by consensus between models, by voting, as a weighted value (e.g., score), as a probability (e.g., by combining probabilities), using a combining model (e.g., a model that takes the outputs from the previous models and outputs a data quality), using a logical operator, according to a prioritization, and/or can otherwise be determined from the plurality of models (e.g., as described for the data quality module). For example, when any of the models outputs a poor data quality (e.g., a bad classification, an unacceptable classification, a quality less than a threshold, etc.), the data can be poor quality (e.g., example shown in FIG. 9). In a first example, each model is evaluated in series. In this example, when one model outputs a poor data quality, the overall data quality can optionally be classified as poor data quality without evaluating the later models in the series (e.g., which can preserve computational resources). In a second example, each model is evaluated in parallel. In a third example, models can be evaluated in parallel and in series. In a specific example, a PG dataset can be first classified with a first data quality as ‘high quality’ (e.g., on a user device) based on a motion model, a body region contact model, and/or a placement model (e.g., parallel models). In this specific example, the high quality PG dataset can then be classified (e.g., on a remote computing system) as ‘low signal quality’ based on a signal quality model (e.g., in series with the motion model, a body region contact model, and/or a placement model). An example is shown in FIG. 23. However, a data quality can otherwise be determined.

High quality data (e.g., a data quality meeting one or more criteria such as: a ‘good’ or acceptable classification, a score that is at least a threshold, a probability of acceptable cardiovascular parameter calculation exceeds a threshold, etc.) is preferably stored and/or used to determine the cardiovascular parameter (e.g., in S300 or S400 such as after enough high quality data has been acquired). When low (or high) quality data (e.g., ‘bad’ or ‘unacceptable’ classification, a score that is at most a threshold, a probability of acceptable cardiovascular parameter calculation is at most a threshold, etc.) is detected (e.g., identified, labeled, etc.), S100 can be performed again (e.g., restarted), high quality data within a threshold distance (e.g., time) of the low quality data can be excluded (e.g., from S300, from S400, from storage, etc.), data can be processed to improve a quality (e.g., using a transformation that converts the data to a higher data quality), a flag can be issued indicating a data quality (e.g., to be attached, appended, or otherwise associated with a cardiovascular parameter determined using the dataset), instructions (e.g., advice or feedback) for improving data quality can be generated and/or presented to the user (and/or operator), and/or any suitable response can occur. However, a data can be used in any manner based on a data quality comparison to one or more criteria.

The method can optionally include guiding a user based on the quality of the data S250 which can function to instruct the user to adjust one or more conditions based on the data quality (e.g., based on an output of the data quality module). Conditions can include: a user, user device, and/or user body region motion; a body region pose relative to a sensor; body region contact pressure; environmental conditions (e.g., ambient light); and/or any other parameter affecting data quality. The user is preferably guided on the user device, but can alternatively be guided on any other suitable system.

The user can be guided based on a data quality using: look-up models, decision trees, rules, heuristics, selection methods, machine learning, regressions, thresholding, classification, equations, probability or other statistical methods, deterministics, genetic programs, support vectors, instance-based methods, regularization methods, Bayesian methods, kernel methods, and/or any other suitable method. In an example, each data quality output from one or more models (e.g., the placement model) in the data quality module is mapped to a user guidance. In an illustrative example, a placement model output of [1,0,0,0,0,0,0,0] results in no guidance (e.g., acceptable body region placement); [0,1,0,0,0,0,0,0] results in ‘lower body region contact pressure’ guidance; [0,0,1,0,0,0,0,0] results in ‘increase body region contact pressure’ guidance; [0,0,0,1,0,0,0,0] results in ‘move body region up’ guidance; [0,0,0,0,1,0,0,0] results in ‘move body region down’ guidance; [0,0,0,0,0,1,0,0] results in ‘move body region left’ guidance; [0,0,0,0,0,0,1,0] results in ‘move body region right’ guidance; [0,0,0,0,0,0,0,1] results in ‘stop moving body region’ guidance.

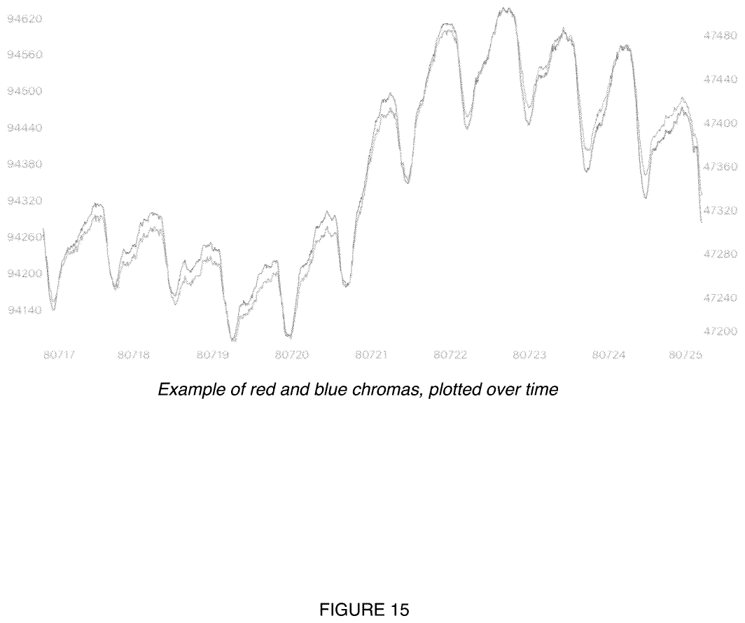

In a first variant, the user can be instructed to decrease motion of the user device in response to a flag outputted from the motion model (e.g., indicating unacceptable conditions). An example is shown in FIG. 17A. In a second variant, the user can be instructed to improve body region contact with the sensor in response to a flag outputted from the body region contact model (e.g., indicating unacceptable conditions). In examples, the user can be instructed to: place the body region on the sensor, adjust positioning of the body region on the sensor, adjust contact pressure, increase blood flow to the body region (e.g., by making a fist), and/or perform any other adjustment. An example is shown in FIG. 17B. In a third variant, the user can be instructed to improve body region placement relative to the sensor (e.g., including pose and/or contact pressure) in response to a flag outputted from the placement model (e.g., indicating unacceptable conditions). In a first example, the user can be instructed to move the body region in a direction (e.g., up, down, left, or right), wherein the direction is based on the placement model output. In an illustrative example, the user is instructed to move their finger to the left when the placement model output indicates the finger is too far to the right of the camera lens center. In a second example, the user can be instructed to adjust contact pressure of the body region on the sensor, wherein the pressure adjustment (e.g., increase vs decrease, the amount of adjustment, etc.) is based on the placement model output. In a fourth variant, the user can be instructed to increase body region temperature (e.g., the body region is too cold) in response to a flag outputted from the signal quality model (e.g., indicating unacceptable signal quality). An example is shown in FIG. 17C. In examples, the user can be instructed to: increase blood flow to the body region, increase temperature of the body region (e.g., by making a fist), and/or perform any other adjustment. An example is shown in FIG. 17C. In a fifth variant, different combinations of data quality module outputs (e.g., classifications) map to different guidance. Additionally or alternatively, a flag from one or more data quality modules can result in discarding the corresponding data sample (e.g., a video acquired via S100 and analyzed via S200) and restarting data acquisition (e.g., all or parts S100), wherein the user can optionally be informed that data acquisition is restarting.

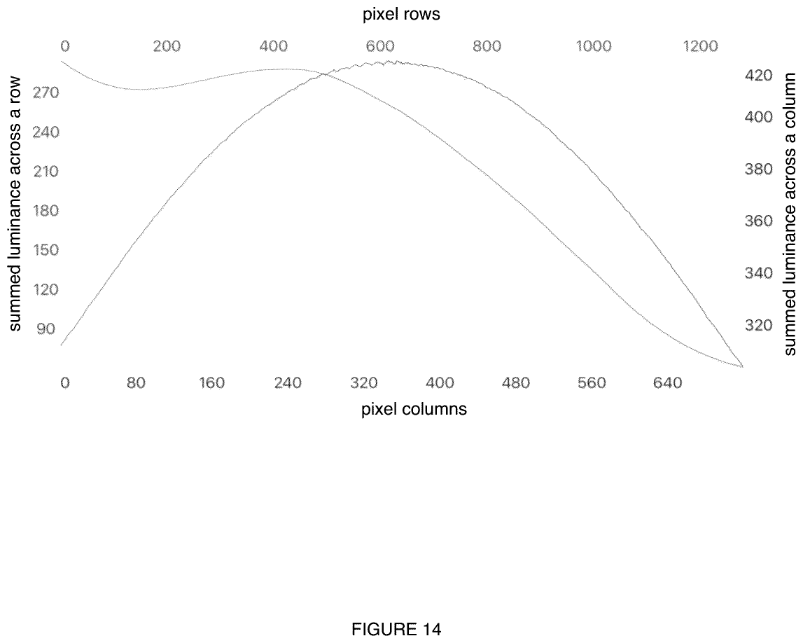

Additionally or alternatively, the user can be guided using a video (e.g., live video) of the body region of the user. An example is shown in FIG. 16A and FIG. 16B.

Additionally or alternatively, S250 can be performed during S100. For example, the user can be guided while acquiring data (e.g., image data, motion data, etc.) to vary a set of conditions (e.g., contact pressure, body region pose including position and/or orientation, user device pose, environmental parameters, etc.). The data quality can be assessed in each of the set of conditions to determine at least one condition associated with data of desired quality. The set of conditions can be a predetermined set of conditions, such that the individual is guided to sequentially vary the conditions; however, the set of conditions can alternatively not be predetermined, such that the individual is able to freely adjust the conditions. S250 can additionally or alternatively include guiding the user to maintain the condition that results in the best data quality.

However, the user can be otherwise guided.

Processing the datasets S300 preferably functions to format and/or analyze the dataset(s) (e.g., to facilitate or enable their use in S400 and/or S500). S300 can be performed by a processing module (e.g., of a local or remote computing system), and/or by any suitable component. S300 can be performed after S100 (e.g., after each segment of data is acquired), after S200 (e.g., after data quality is determined for each segment of data), and/or at any other time. The datasets processed in S300 are preferably data used in (e.g., validated in) S200, but can alternatively be a subset of data used in S200, a superset of data used in S200, and/or entirely different from data used in S200. S300 preferably processes data with high quality (e.g., ‘good’ data), but can process low quality, data without a quality, and/or any suitable quality data.

Examples of processing the datasets can include: aggregating datasets; removing outliers, averaging (e.g., using a moving average) the datasets, converting an image set to PG data (e.g., by averaging or summing intensity of images of the image set, using a transformation, otherwise generating a PG dataset, etc.), resampling the datasets; filtering the datasets; segmenting the datasets (e.g., into heartbeats); denoising the datasets; determining a subset of the datasets to analyze; and/or otherwise processing the datasets.

S300 preferably processes at least a threshold number of seconds worth of data, but can alternatively process any number of seconds worth of data and/or process data not associated with a time window. The threshold number of seconds (e.g., prior to aggregating datasets) can be between 0.5 s-600 s or any range or value therebetween (e.g., 0.5 s, 1 s, 2 s, 4 s, 5 s, 8 s, 10 s, 12 s, 15 s, 20 s, 25 s, 50 s, loos, etc.), but can alternatively be less than 0.5 s or greater than 600 s. Aggregating datasets can optionally include accumulating data segments to generate a threshold amount of data (e.g., a threshold number of seconds worth of data). The threshold number of seconds can be between 4 s-600 s or any range or value therebetween (e.g., 5 s, 8 s, 10 s, 12 s, 15 s, 20 s, 25 s, 50 s, loos, etc.), but can alternatively be less than 4 s or greater than 600 s. The data (e.g., aggregated data) can be contiguous (e.g., PG data extracted from an uninterrupted segment of a video) or noncontiguous (e.g., PG data extracted from discrete, non-neighboring segments of a video).

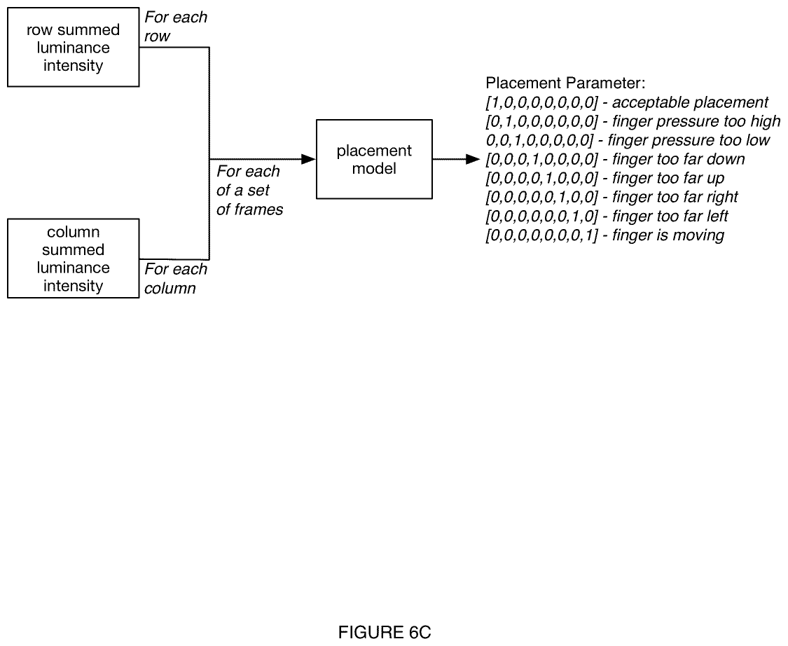

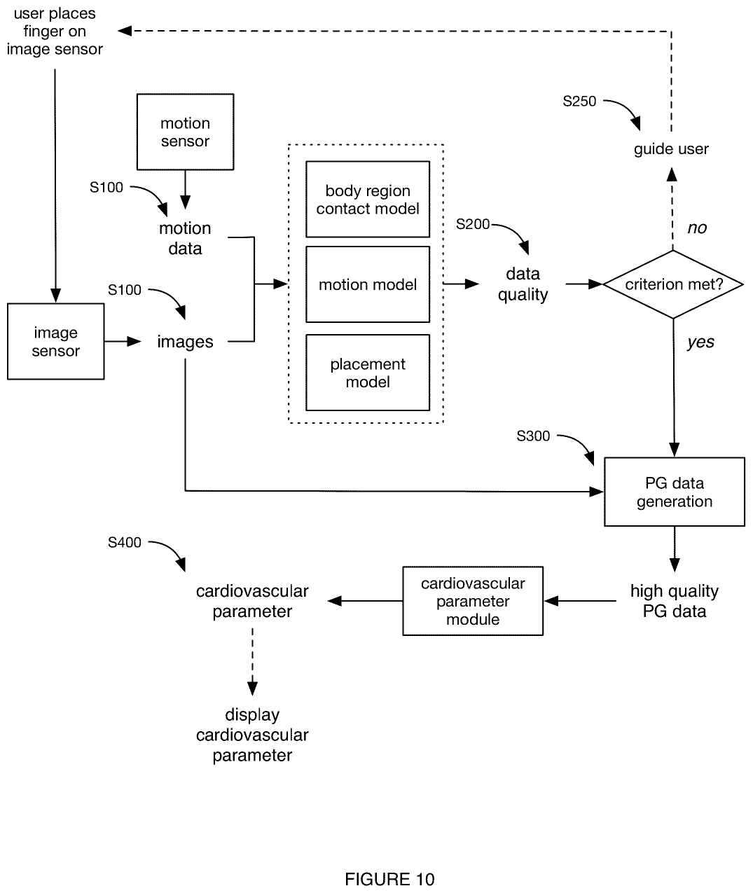

In an example of aggregating datasets, consecutive or nonconsecutive segments of data can be accumulated to generate a timeseries of aggregated data, wherein the length of the timeseries of aggregated data can be substantially equal to the threshold length of time (e.g., as described for data inputs to the cardiovascular parameter module). In an illustrative example, a first segment of data is acquired (e.g., a first video) via S100 methods, wherein data quality associated with the first segment is classified via S200 methods. If the data quality classification is ‘bad’, the first segment is discarded and data accumulation restarts. If the data quality classification is ‘good’, a second segment of data is acquired (e.g., a second video, consecutive with the first video) via S100 methods, wherein data quality associated with the second segment is classified via. S200 methods. If the data quality classification associated with the second segment is ‘good’, the second segment is appended to the first segment to generate an aggregated timeseries. If the data quality classification associated with the second segment is ‘bad’, either: both segments of data can be discarded and data accumulation restarts (e.g., such that the final aggregated timeseries is contiguous); or only the second segment is discarded and the data accumulation method resumes for a new second segment (e.g., such that the final aggregated timeseries is noncontiguous). Subsequent segments can be iteratively appended until the aggregated timeseries reaches a threshold length of time. Examples are shown in FIG. 8 and FIG. 12.

Processing the datasets can be performed, in a first example, in a manner as disclosed in U.S. patent application Ser. No. 17/761,152 titled ‘METHOD AND SYSTEM FOR DETERMINING CARDIOVASCULAR PARAMETERS’ filed on 16 Mar. 2022 which is incorporated in its entirety by this reference. Processing the datasets can be performed, in a second example, as disclosed in U.S. patent application Ser. No. 17/866,185 titled ‘METHOD AND SYSTEM FOR CARDIOVASCULAR DISEASE ASSESSMENT AND MANAGEMENT’ filed on 15 Jul. 2022 which is incorporated in its entirety by this reference. However, processing the datasets can be performed in any manner.

Determining the cardiovascular parameter(s) S400 functions to evaluate, calculate, estimate, and/or otherwise determine the user's cardiovascular parameters from the PG dataset (e.g., processed PG dataset, denoised PG dataset, segmented PG dataset, filtered PG dataset, interpolated PG dataset, raw PG dataset, etc.). S400 can additionally or alternatively function to determine fiducials (and/or any other suitable parameters) associated with the cardiovascular parameters of the individual. The user's cardiovascular parameters are preferably determined using high quality datasets (e.g., high quality PG data), but can be determined using low quality datasets (e.g., with or without reporting an estimated error from using lower quality data, with or without including a flag indicating that potentially faulty data has been used, etc.), using a combination of high and low quality datasets, and/or using any suitable data. S400 is preferably performed using a cardiovascular parameter module (e.g., of a computing system such as a local or remote computing system), but can be performed by any suitable component. The PG dataset is preferably transformed (e.g., using a linear transformation, using a nonlinear transformation, etc.) into the cardiovascular parameter. However, additionally, or alternatively, any suitable dataset can be used (e.g., used to calculate) and/or transformed into the cardiovascular parameter.

Determining the cardiovascular parameter can include analyzing the PG dataset (e.g., an analysis PG dataset). The PG dataset can be analyzed on a per segment basis (e.g., cardiovascular parameters determined for each segment), for the PG dataset as a whole, for an averaged PG dataset, and/or otherwise be analyzed. S400 is preferably performed independently for each segment of the PG dataset; however, S400 can be performed for the entire PG dataset, the analysis of one segment can depend on the results of other segments, and/or any suitable subset of the PG dataset can be analyzed.

The cardiovascular parameter(s) can be determined based on the PG dataset, fiducials, and/or cardiovascular manifold using regression modeling (e.g., linear regression, nonlinear regression, generalized linear model, generalized additive model, etc.), learning (e.g., a trained neural network, a machine-learning algorithm, etc.), an equation, a look-up table, conditional statements, a transformation (e.g., a linear transformation, a non-linear transformation, etc.), and/or determined in any suitable manner.

The transformation (e.g., correlation) between the fiducials and/or the cardiovascular manifold and the cardiovascular parameters is preferably determined based on a calibration dataset (e.g., a calibration dataset such as from a blood pressure cuff, ECG measurements, etc. generated at approximately the same time as the analysis PG dataset; a second PG dataset such as at a different body region of the individual, of a different individual, of the individual in a different activity state, etc.; a calibration dataset including an analysis PG dataset for each individual of a control group with a corresponding measured cardiovascular parameter; etc.); however, the transformation can be determined from a model (e.g., a model of the individual's cardiovascular system, a global model such as one that can apply for any user, etc.), and/or determined in any suitable manner.

In variants, S400 can include: determining fiducials; determining cardiovascular parameters; and storing the cardiovascular parameters. However, S400 can include any suitable processes.

Determining fiducials preferably functions to determine fiducials for the PG dataset (e.g., processed dataset, denoised dataset, segmented dataset, filtered dataset, interpolated dataset, raw dataset, etc.). This preferably occurs before determining the cardiovascular parameters; however, the fiducials can be determined at the same time as and/or after cardiovascular parameter determination. The set of fiducials can depend on the cardiovascular parameters, characteristics of the individual, a supplemental dataset, and/or any suitable information. In some variants, different fiducials can be used for different cardiovascular parameters; however, two or more cardiovascular parameters can be determined from the same set of fiducials.

In a first variant, determining the fiducials can include decomposing the PG dataset (e.g., for each segment in the analysis PG dataset) into any suitable basis function(s). In a specific example, decomposing the PG dataset can include performing a discrete Fourier transform, fast Fourier transform, discrete cosine transform, Hankel transform, polynomial decomposition, Rayleigh, wavelet, and/or any suitable decomposition and/or transformation on the PG dataset. The fiducials can be one or more of the decomposition weights, phases, and/or any suitable output(s) of the decomposition. However, the fiducials can be determined from the PG dataset in any suitable manner.

In a second variant, determining the fiducials can include fitting the PG dataset to a predetermined functional form. The functional form can include radial basis functions (e.g., gaussians), Lorentzians, exponentials, super-gaussians, Lévy distributions, hyperbolic secants, polynomials, convolutions, linear and/or nonlinear combinations of functions, and/or any suitable function(s). The fitting can be constrained or unconstrained. In a first specific example, a linear combination of 5 constrained gaussians (e.g., based on user's cardiovascular state and/or phase) can be used to fit each segment of the PG data. In a second specific example, a linear combination of 4 gaussians can be fit to each segment of the PG data. The 4 gaussians can represent: a direct arterial pressure model, two reflected arterial pressure models, and a background model (e.g., where the background is a slow moving gaussian for error correction). However, any other number of gaussians, representing any other suitable biological parameter, can be fit (e.g., concurrently or serially) to one or more segments of the PG data.

The functional form can be fit to the PG dataset based on: a loss between the functional form and the PG dataset, a loss between derivatives of the functional form and derivatives of the PG dataset (e.g., first derivative, second derivative, third derivative, a weighted combination of derivatives, etc.), and/or any other fitting methods. In an illustrative example, a linear combination of gaussians are simultaneously fit to a segment of the PG data to minimize loss between the first, second, and third derivative of the linear combination of gaussians relative to the first, second, and third derivative of the PG data segment, respectively. The fitting can be multi-stage or single-stage. In a specific example of multi-stage fitting, the first fitting stage includes determining a timing parameter (e.g., spacing between gaussians, frequency, center position and/or any other model location, ordinal, etc.) of each gaussian in a linear combination of gaussians by minimizing loss between the first and/or second derivative of the linear combination of gaussians relative to the first and/or second derivatives of the PG data segment, respectively. The second fitting stage includes determining an amplitude parameter (e.g., the amplitude, a parameter in the gaussian function that influences the amplitude, a parameter based on the amplitude, etc.) of each gaussian in the linear combination by minimizing loss between the third derivative of the linear combination of gaussians relative to the third derivative of the PG data segment. In this second stage, the timing parameter for each gaussian can be substantially constrained.

However, any suitable fit can be performed.

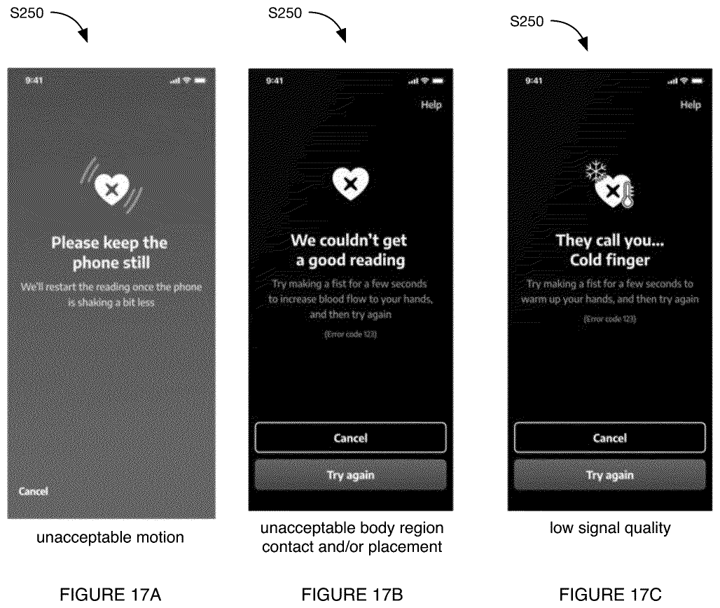

In this variant, the fiducials are preferably one or more of the fit parameters (e.g., full width at half max (FWHM), center position, location, ordinal, amplitude, frequency, spacing, any timing parameter, any amplitude parameter, etc.); however, the fiducials can include statistical order information (e.g., mean, variance, skew, etc.) and/or any suitable information. An example is shown in FIG. 19.

Determining the cardiovascular parameters preferably functions to determine the cardiovascular state (e.g., set of cardiovascular parameter values) for the user. The cardiovascular parameters can be determined based on the fiducials (e.g., for a single segment; for the entire PG dataset, wherein corresponding fiducials are aggregated across the segments; etc.), based on the cardiovascular manifold, and/or otherwise be determined. This preferably determines cardiovascular parameters relating to each segment of the PG dataset (e.g., each heartbeat); however, this can determine a single cardiovascular parameter value for the entire PG dataset (e.g., a mean, variance, range, etc.), a single cardiovascular parameter, and/or any suitable information. This preferably occurs before storing the cardiovascular parameters; however, S436 can occur simultaneously with and/or after storing the cardiovascular parameters.

In a first variant, the cardiovascular parameters can be determined by applying a fiducial transformation to the set of fiducials. The fiducial transformation can be determined from a calibration dataset (e.g., wherein a set of fiducial transforms for different individuals are determined by multiplying the cardiovascular parameters by the inverse matrix of the respective fiducials), based on a model (e.g., a model of the individual, a model of human anatomy, a physical model, etc.), generated using machine learning (e.g., a neural network), generated from a manifold (e.g., relating fiducial value sets with cardiovascular parameter value sets), based on a fit (e.g., least squares fit, nonlinear least squares fit, generalized linear model, generalized additive model, etc.), and/or be otherwise determined. The fiducial transformation can be a universal transformation, be specific to a given cardiovascular parameter or combination thereof, be specific to the individual's parameters (e.g., age, demographic, comorbidities, biomarkers, medications, estimated or measured physiological state, etc.), be specific to the individual, be specific to the measurement context (e.g., time of day, ambient temperature, etc.), or be otherwise generic or specific. The fiducial transformation can be the average, median, most accurate (e.g., lowest residuals, lowest error, etc.), based on a subset of the control group (e.g., a subset of the control group with one or more characteristics similar to or matching the individual's characteristics), selected based on voting, selected by a neural network, randomly selected, and/or otherwise determined from the calibration dataset. The fiducial transformation can be normalized, wherein the fiducial values and/or the cardiovascular parameter values used to determine the transformation are demeaned and/or otherwise modified.

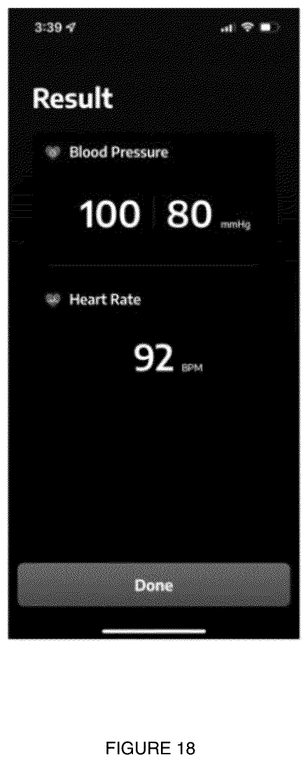

The fiducial transformation can be a linear or nonlinear transformation. In an example, the fiducial transformation is a linear transformation of a synthetic fiducial, wherein the synthetic fiducial is a combination (e.g., linear combination, nonlinear combination, etc.) of the set of fiducials. In this example, the transformation can be determined based on a generalized additive model fit to a calibration dataset including cardiovascular parameters and a set of fiducial values corresponding to each cardiovascular parameter (e.g., where the link function of the generalized additive model is the transformation of the synthetic fiducial, where the predictor of the generalized additive model is the synthetic fiducial). An example is shown in FIG. 20. In an illustrative example, determining cardiovascular parameters can include: calculating a synthetic fiducial from the set of fiducials (e.g., using a weighted sum of the fiducials, etc.); and determining a relationship (e.g., linear relationship) between the synthetic fiducial and the cardiovascular parameter. This can be used to determine the universal relationship, manifold, or model (e.g., reference relationship); an individual's relationship, manifold, or model; and/or any other relationship, manifold, or model. However, the fiducial transformation can be otherwise applied.

Each cardiovascular parameter can be associated with a different fiducial transformation and/or one or more cardiovascular parameters can be associated with the same fiducial transformation (e.g., two or more cardiovascular parameters can be correlated or covariate). In a specific example of the first variant, the cardiovascular parameters can be determined according to:

AT=B

where A corresponds to the set of fiducials, T corresponds to the fiducial transformation, and B corresponds to the cardiovascular parameter(s).

In a specific example, the method includes: determining the fiducial transformation for an individual, and determining the cardiovascular parameter value(s) for the individual based on a subsequent cardiovascular measurement and the fiducial transformation. The fiducial transformation is preferably determined from a set of calibration data sampled from the individual, which can include: fiducials extracted from calibration cardiovascular measurements (e.g., PG data, plethysmogram data) (A), and calibration cardiovascular parameter measurements (e.g., blood pressure, O₂ levels, etc.; measurements of the cardiovascular parameter to be determined) (B). The fiducial transformation (T) for the individual is determined from AT=B. T is subsequently used to determine the cardiovascular parameter values for fiducials extracted from subsequently-sampled cardiovascular measurements.

In a second variant, the cardiovascular parameters can be determined based on where the individual is on the individual's cardiovascular manifold, a manifold transformation from the individual's cardiovascular manifold to a universal cardiovascular manifold, and optionally a mapping transformation from the individual's position on the universal cardiovascular manifold to the cardiovascular parameter values. The cardiovascular parameter can additionally or alternatively depend on a change in where the individual is on the cardiovascular manifold (e.g., a change in fiducial values, a change in a cardiovascular parameter, etc.), the individual's effective location on the universal cardiovascular manifold (e.g., a normalized universal cardiovascular manifold), the change in the individual's effective location on the universal cardiovascular manifold, and/or otherwise depend on the individual's relationship to the cardiovascular manifold. The universal cardiovascular manifold can be determined from the calibration dataset, determined from a model, generated using machine learning (e.g., a neural network), and/or be otherwise determined. The universal cardiovascular manifold can be an average of, include extrema of, be learned from (e.g., using machine learning algorithm to determine), be selected from, and/or otherwise be determined based on the calibration dataset. The universal cardiovascular manifold preferably maps values for one or more fiducials to values for cardiovascular parameters, but can be otherwise constructed. The universal cardiovascular manifold preferably encompasses at least a majority of the population's possible fiducial values and/or cardiovascular parameter values, but can encompass any other suitable swath of the population. The universal cardiovascular manifold can be specific to one or more cardiovascular parameters (e.g., the system can include different universal manifolds for blood pressure and oxygen levels), but can alternatively encompass multiple or all cardiovascular parameters of interest. The manifold transformation can include one or more affine transformation (e.g., any combination of one or more: translation, scaling, homothety, similarity transformation, reflection, rotation, and shear mapping) and/or any suitable transformation. In an illustrative example of the second variant, the individual's cardiovascular phase can be determined and aligning (e.g., using a transformation) the individual's cardiovascular phase to a universal cardiovascular phase (e.g., associated with a universal cardiovascular manifold), where a relationship between the universal cardiovascular phase and the cardiovascular parameters is known.

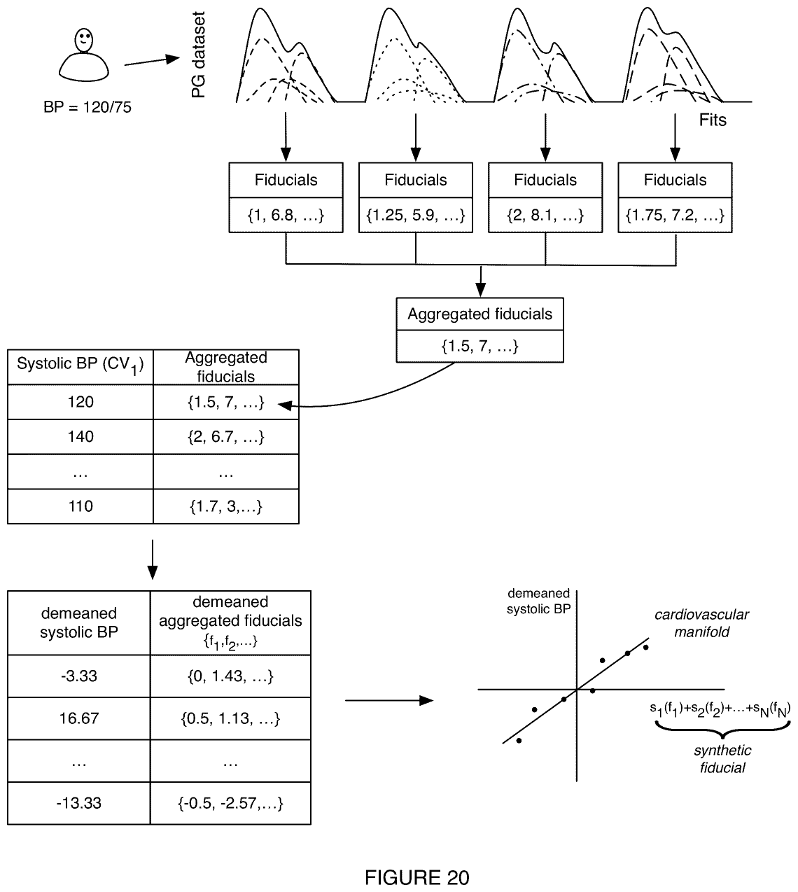

In a first specific example, the method includes: generating the universal manifold from population calibration data, generating an individual manifold from an individual's calibration data, and determining a transformation between the individual manifold and the universal manifold. The universal manifold is preferably a finite domain and encompasses all (or a majority of) perturbations and corresponding cardiovascular parameter values (e.g., responses), but can encompass any other suitable space. The universal manifold preferably relates combinations of fiducials (with different values) with values for different cardiovascular parameters (e.g., relating one or more reference sets of fiducials and one or more reference cardiovascular parameters), but can relate other variables. The individual calibration data preferably includes cardiovascular measurements (e.g., PG data, plethysmogram data) corresponding to cardiovascular parameter measurements (e.g., blood pressure), but can include other data. The population calibration data preferably includes data similar to the individual calibration data, but across multiple individuals (E.g., in one or more physiological states). The transformation can be: calculated (e.g., as an equation, as constants, as a matrix, etc.), estimated, or otherwise determined. The transformation preferably represents a transformation between the individual and universal manifolds, but can additionally or alternatively represent a mapping of the fiducial position on the universal manifold (e.g., the specific set of fiducial values, transformed into the universal domain) to the cardiovascular parameter values (e.g., in the universal domain). Alternatively, the method can apply a second transformation, transforming the universal-transformed fiducial values to the cardiovascular parameter values (e.g., in the universal domain). The transformation(s) are subsequently applied to the fiducials extracted from subsequent cardiovascular measurements from the individual to determine the individual's cardiovascular parameter values. The transformation can optionally be between normalized manifolds, wherein a normalized manifold can include a relationship between cardiovascular parameters and fiducials determined based on demeaned cardiovascular parameters (e.g., subtracting a cardiovascular parameter offset, wherein the cardiovascular parameter offset is defined as the average of the cardiovascular parameters) and demeaned fiducials (e.g., wherein a fiducial offset is subtracted from the synthetic fiducials; wherein a fiducial offset is subtracted from values for each fiducial, etc.); an example is shown in FIG. 22.

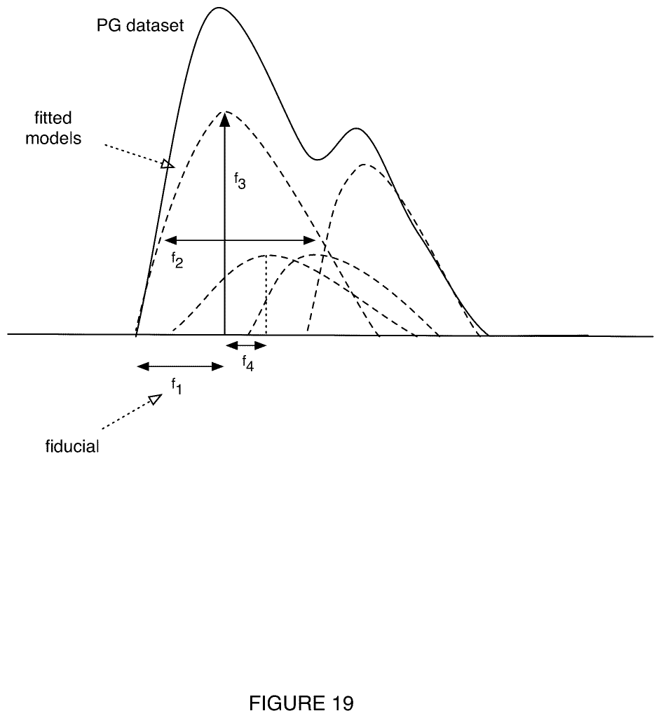

In a second specific example, the method includes: generating the universal manifold from population calibration data, determining a set of offsets for an individual manifold based on an individual's calibration data, determining a change in fiducial values for the individual, determining a cardiovascular parameter change based on the normalized universal manifold and the set of offsets, and calculating the cardiovascular parameter for the individual based on the cardiovascular parameter change. The universal manifold (e.g., reference relationship between one or more reference sets of fiducials and one or more reference cardiovascular parameters) is preferably normalized with respect to a baseline (e.g., a mean cardiovascular parameter and a mean set of fiducials and/or synthetic fiducial), but can be non-normalized and/or otherwise processed. The baseline can be determined using (e.g., averaging) measurements recorded during a rest state of one or more individuals, using a set of measurements recorded across a set of cardiovascular states for one or more individuals, and/or using measurements recorded during any other state. The set of offsets for the individual manifold (e.g., individual relationship) preferably includes one or more fiducial offsets (e.g., wherein the fiducial offset can be the average of the synthetic fiducials, the average values for each fiducial, etc.) and/or a cardiovascular parameter offset (e.g., the average of the cardiovascular parameters). The set of offsets can be determined based on a single calibration datapoint (e.g., while the individual is at rest) and/or multiple calibration datapoints. A change in fiducial values for the individual can be determined based on a PG dataset (e.g., a non-calibration dataset), or otherwise determined. The change can be relative to the fiducial offset and/or relative to another fiducial reference. The corresponding cardiovascular parameter change can be determined based on the (normalized) universal manifold prescribing a relationship between changes in fiducials (e.g., individual fiducials, synthetic fiducials, etc.) and changes in the cardiovascular parameter. The relationship can be a fiducial transformation (e.g., as previously described for a universal cardiovascular manifold), can be based on a fiducial transformation (e.g., the slope of a linear transformation between a synthetic fiducial and cardiovascular parameter), can be a relationship (e.g., a 1:1 mapping) between fiducials (e.g., individual fiducials and/or fiducial sets) and cardiovascular parameter measurements (e.g., individual measurements and/or sets of measurements; measured for one or more individuals), and/or can be otherwise defined. The cardiovascular parameter for the individual can be calculated by summing: the cardiovascular parameter change, the cardiovascular parameter offset, and/or a cardiovascular parameter reference (e.g., a cardiovascular parameter corresponding to the fiducial reference). An example is shown in FIG. 21. Additionally or alternatively, the individual's cardiovascular parameter value can be determined by calculating a universal fiducial value corresponding to the individual's fiducial value (e.g., based on the fiducial change and the fiducial offset), and identifying the universal cardiovascular parameter value on the universal manifold corresponding to the universal fiducial value. The universal cardiovascular parameter value can optionally be corrected by the individual's cardiovascular parameter offset. However, the cardiovascular parameter can be otherwise determined.

Embodiments of determining cardiovascular parameters can include determining a cardiovascular manifold for the individual. For example, an individual's cardiovascular manifold can correspond to a surface relating the individual's heart function, nervous system, and vessel changes. In a specific example, a cardiovascular manifold can map fiducial values to corresponding cardiovascular parameter values and nervous system parameter values (e.g., parasympathetic tone, sympathetic tone, etc.). However, the cardiovascular manifold can additionally or alternatively depend on the individual's endocrine system, immune system, digestive system, renal system, and/or any suitable systems of the body. The cardiovascular manifold can additionally or alternatively be a volume, a line, and/or otherwise be represented by any suitable shape. The individual's cardiovascular manifold is preferably substantially constant (e.g., slowly varies such as does not differ day-to-day, week-to-week, month-to-month, year-to-year, etc.) across the individual's lifespan. As such, an individual's cardiovascular manifold can be stored to be accessed at and used for analyzing the individual's cardiovascular parameters at a later time. However, an individual's cardiovascular manifold can be variable and/or change considerably (e.g., as a result of significant blood loss, as a side effect of medication, etc.) and/or have any other characteristic over time.

In some variants, the cardiovascular manifold can correspond to and/or be derived from the predetermined functional form (e.g., from the third variant of fiducial determination). However, the cardiovascular manifold can be otherwise related to and/or not related to the fiducials.

The cardiovascular manifold preferably corresponds to a hyperplane, but can additionally or alternatively correspond to a trigonometric manifold, a sigmoidal manifold, hypersurface, higher-order manifold, and/or be described by any suitable topological space.

For example, determining the cardiovascular manifold for the individual can include fitting each of a plurality of segments of a PG dataset (e.g., segmented dataset, processed dataset, subset of the dataset, etc.) to a plurality of gaussian functions such as,

$$\hat{f} ( t ) = {\sum }_{i}^{N} {p}_{{a}_{i}} ( < φ > ) e \frac{{( {p}_{{b}_{i}} ( < φ > ) - t )}^{2}}{{p}_{{c}_{i}} ( < φ > )}$$

Where \hat{f}(t) is the segment of the PG dataset, t is time, N is the total number of functions being fit, i is the index for each function of the fit; a,b, and c are fit parameters, and pₓ_{ᵢ} are functions of the cardiovascular phase <φ> where the fit parameters are constrained to values of pₓ_{ᵢ}. The constraining functions can be the same or different for each fit parameter. The constraining functions are preferably continuously differentiable, but can be continuously differentiable over a predetermined time window and/or not be continuously differentiable. Examples of constraining functions include: constants, linear terms, polynomial functions, trigonometric functions, exponential functions, radical functions, rational functions, combinations thereof, and/or any suitable functions.

In a third variant, determining the cardiovascular parameters can include determining the cardiovascular parameters based on the supplemental data. For example, the fiducial transformation and/or manifold transformation can be modified based on the supplemental data (such as to account for a known bias or offset related to an individual's gender or race). Examples of supplemental dataset can include: characteristics of the individual (e.g., height, weight, age, gender, race, ethnicity, etc.), medication history of the individual (and/or the individual's family), activity level (e.g., recent activity, historical activity, etc.) of the individual, medical concerns, healthcare profession data (e.g., data from a healthcare professional of the individual), and/or any suitable supplemental dataset.

In a fourth variant, the cardiovascular parameters can be determined in more than one manner. For example, the cardiovascular parameters can be determined according to two or more of the above variants. In the fourth variant, the individual cardiovascular parameters can be the average cardiovascular parameter, the most probable cardiovascular parameters, selected based on voting, the most extreme cardiovascular parameter (e.g., highest, lowest, etc.), depend on previously determined cardiovascular parameters, and/or otherwise be selected.

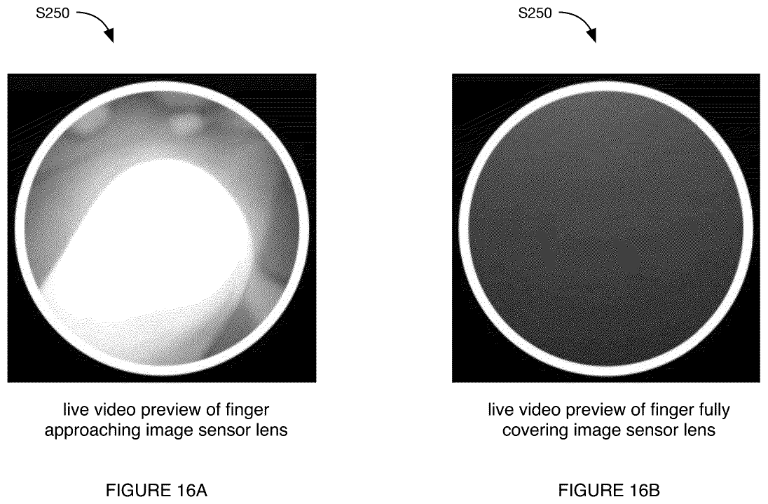

The cardiovascular parameter can optionally be: presented to the user (e.g., displayed at the user device; example shown in FIG. 18), provided to a care provider and/or guardian, used to determine a health assessment of the user (e.g., an assessment of cardiovascular disease such as hypertension, atherosclerosis, narrow of blood vessels, arterial damage, etc.), used to calibrate the cardiovascular parameter module (e.g., when compared to a cardiovascular parameter determined via a blood pressure cuff and/or any other system), and/or otherwise used. Additionally or alternatively, communication between the user and a healthcare provider can be initiated (e.g., automatically initiated) and/or otherwise facilitated based on the cardiovascular parameter, a treatment can be administered (e.g., automatically administered) based on the cardiovascular parameter, a treatment plan can be determined (e.g., automatically determined) based on the cardiovascular parameter, and/or the cardiovascular parameter can be otherwise used.

The cardiovascular parameter can be determined, in a first example, in a manner as disclosed in U.S. patent application Ser. No. 17/711,897 titled ‘METHOD AND SYSTEM FOR DETERMINING CARDIOVASCULAR PARAMETERS’ filed on 1 Apr. 2022 which is incorporated in its entirety by this reference. The cardiovascular parameter can be determined, in a second example, in a manner as disclosed in U.S. patent application Ser. No. 17/761,152 titled ‘METHOD AND SYSTEM FOR DETERMINING CARDIOVASCULAR PARAMETERS’ filed 16 Mar. 2022, which is incorporated in its entirety by this reference. The cardiovascular parameter can be determined, in a third example, in a manner as disclosed in U.S. patent application Ser. No. 17/588,080 titled ‘METHOD AND SYSTEM FOR ACQUIRING DATA FOR ASSESSMENT OF CARDIOVASCULAR DISEASE’ filed 28 Jan. 2022, which is incorporated in its entirety by this reference.

However, the cardiovascular parameter(s) can otherwise be determined.

Training a data quality module S500 functions to train one or more models in the data quality module (e.g., wherein the trained models can be implemented locally on the user device). S500 can be performed prior to: S100, S200, S300, and/or S400; and/or at any other time.

When more than one model is used (e.g., in a single data quality module, across multiple data quality modules, etc.), each model is preferably independently trained, but alternatively can be dependently trained. The same training data can be used to train different models and/or different training data can be used to train the models. For example, the same training data can be to train (e.g., independently train) a body region contact model and a placement model.

Training a data quality module can include: acquiring training data (e.g., via S100) with a set of training users under a first set of conditions (e.g., acceptable conditions, corresponding to one or more acceptable labels) and under a second set of conditions (e.g., unacceptable conditions, corresponding to one or more unacceptable labels), wherein the data quality module (e.g., a model in the data quality module) is trained to predict a label based on the training data (e.g., attributes extracted from the training data). The training data can optionally include overlapping time windows of data (e.g., to increase the amount of training data). The training data preferably includes data segments with the same size (e.g., same number of frames) as used in S200, but can alternatively be data of any size. The data segments preferably include the same type of data as that used in S200, but can additionally or alternatively include more or less data.

The labels are preferably binary (e.g., ‘acceptable’ or ‘unacceptable’), but can alternatively be multiclass, a value (e.g., discrete, continuous, etc.), and/or any other label. In an example of multiclass labels, the labels can indicate a specific acceptable or unacceptable condition. In an illustrative example, the labels can be body region pose labels: ‘too far left,’ ‘too far right,’ ‘too far up,’ ‘too far down,’ and/or ‘acceptable body region position.’ In a second illustrative example the labels can be body region contact pressure labels: “pressure too low,” “pressure too high,” and/or “acceptable pressure.” However, the labels can be otherwise configured. The labels can be: manually assigned, assigned based on the instructions given to the training user, determined using a secondary model, and/or otherwise determined.

In a first variant, the sets of conditions (e.g., acceptable and unacceptable conditions) are predetermined conditions. For example, acceptable and unacceptable conditions can be determined based on thresholds associated with the sensor. In a second variant, the sets of conditions can be empirically determined (e.g., during training, after training, during model testing, based on user testing, etc.). When more than one model is used, each model can be trained using the same or different sets of conditions. Acceptable and/or unacceptable conditions can optionally include multiple user devices (e.g., multiple makes and models), multiple environmental conditions (e.g., ambient light conditions), multiple user parameters, and/or any other parameters.

In an example of training a motion model, acceptable conditions can include: the user remaining seated and still; minimizing user device and/or user (e.g., body, arm, hand, and/or finger) movements during the measurement period (e.g., small device movement, device movement below a threshold motion, etc.); and/or any other conditions that facilitate high data quality. In specific examples, acceptable conditions can include: alternative user wrist poses (e.g., wherein the user device pose is based on the user wrist pose), slowly rotating and/or adjusting the user wrist, slight forearm movement and/or adjustment (e.g., up or down), slight user and/or user device bounce, slight user and/or user device movement due to breathing, talking and/or yelling, and/or any other acceptable pose and/or movement conditions. Unacceptable conditions can include: the user not remaining seated and/or still; the user and/or user device moving during the measurement period beyond a reasonable amount (e.g., beyond a threshold linear acceleration, angular acceleration, jerk, etc.); and/or any other condition that can lower data quality. In specific examples, unacceptable conditions can include: shaking the user device, rolling and/or rotating the user device, tapping the user device, lifting the body region on and off the sensor, swinging the user arm, raising and lowering the user arm, bouncing the user arm and/or hand, walking, running, squatting, spinning, jumping, going up and/or down stairs, getting up and/or sitting down, shaking (e.g., the user and/or user device), and/or any other unacceptable pose and/or movement conditions.

In an example of training a body region contact model, acceptable conditions can include: proper body region pose (position and/or orientation) relative to the sensor, proper contact pressure between the body region and the sensor, proper movement of the body region and/or user device (e.g., below a threshold motion), and/or any other conditions that facilitate high data quality. In a first specific example, acceptable conditions can include multiple body region orientations relative to the sensor (e.g., 0°, 45°, 90°, 135°, 180°, 225°, 270°, 315°, any number of degrees in the plane of the image sensor lens, etc.). In a second specific example, acceptable conditions can include a contact pressure 1 oz-50 oz or any range or value therebetween (e.g., 2 oz-15 oz, 3 oz-10 oz, 4 oz-10 oz, the weight of the user device, etc.), but can alternatively include a contact pressure less than 1 oz or greater than 50 oz. Unacceptable conditions can include: improper body region pose relative to the sensor, improper contact pressure between the body region and the sensor, improper movement of the body region and/or user device (e.g., above a threshold motion), and/or any other conditions that can lower data quality. In a first specific example, unacceptable conditions include contact pressure too soft (e.g., hovering; below a first threshold contact pressure value) or too hard (e.g., squishing; above a second threshold contact pressure value). The first contact pressure threshold value can be between 1 oz-5 oz or any range or value therebetween, but can be less than 1 oz or greater than 5 oz. The second contact pressure threshold value can be between 5 oz-50 oz or any range or value therebetween, but can be less than 5 oz or greater than 50 oz. In a second specific example, the body region can be askew from covering the center of the sensor (e.g., too far in any direction, including left, right, up, down, any diagonal, etc.). The body region (e.g., the center of the body region) can be greater than a threshold value askew (in a given direction), wherein the threshold value askew can be between 1 mm-10 mm or any range or value therebetween (e.g., 1 mm, 2 mm, 3 mm, 4 mm, 5 mm, 6 mm, 7 mm, 8 mm, 9 mm, 10 mm, etc.), but can alternatively be less than 1 mm or greater than 10 mm. In other specific examples, unacceptable conditions include: body region movement (e.g., tapping finger on and off the image sensor; tapping the image sensor with the body region to mimic the appearance of heart beats in terms of light intensity changes and/or otherwise moving the finger; etc.), a foreign material or other obstruction between the body region and the sensor (e.g., Band-Aid™ or other bandage, paper, adhesive, clothing or other fabric, etc.), any other user body region (e.g., head, fingernail, etc.) on the sensor that is not a proper body region for the sensor (e.g., finger), a foreign material contacting the sensor instead of the body region (e.g., static and/or with movement; materials can include colored paper, a table, carpet, etc.), lighting (e.g., constant exposure to various lighting conditions), and/or any other unacceptable conditions.

In an example of training a placement model, acceptable conditions can be proper body region pose (position and/or orientation) relative to the sensor, proper contact pressure between the body region and the sensor, proper movement of the body region and/or user device (e.g., below a threshold motion), and/or any other conditions that facilitate high data quality. The acceptable conditions for placement model training are preferably the same as body region contact model and/or motion model acceptable conditions, but can alternatively be different than the body region contact model and/or motion model acceptable conditions. Unacceptable conditions can include: improper body region pose relative to the sensor, improper contact pressure between the body region and the sensor, improper movement of the body region and/or user device (e.g., above a threshold motion), and/or any other conditions that can lower low data quality. In a first specific example, the body region can be askew from covering the center of the sensor (i.e. too far in any direction, including left, right, up, down, any diagonal, etc.). The body region (e.g., the center of the body region) can be greater than a threshold value askew (in a given direction), wherein the threshold value askew can be between 1 mm-10 mm or any range or value therebetween (e.g., 1 mm, 2 mm, 3 mm, 4 mm, 5 mm, 6 mm, 7 mm, 8 mm, 9 mm, 10 mm, etc.), but can alternatively be less than 1 mm or greater than 10 mm. In a second specific example, unacceptable conditions include contact pressure too soft (e.g., hovering; below a first threshold contact pressure value) or too hard (e.g., squishing; above a second threshold contact pressure value). The first contact pressure threshold value can be between 1 oz-5 oz or any range or value therebetween, but can be less than 1 oz or greater than 5 oz. The second contact pressure threshold value can be between 5 oz-50 oz or any range or value therebetween, but can be less than 5 oz or greater than 50 oz. In other specific examples, unacceptable conditions can include: user (e.g., body region) and/or user device movement (e.g., not enough device movement for the motion model to detect; tapping and/or any other movement) no body region contact with the sensor (e.g., sensor exposed to open air, sensor contact with a variety of materials with and/or without movement, etc.) and/or any other unacceptable conditions (e.g., used for the motion model and/or the body region contact model).

The data quality module can optionally be trained using synthetic training data. For example, synthetic training data for a target user device (e.g., a target make and/or model) can be generated using models (e.g., physical models) of the target user device (e.g., based on non-synthetic training data for an initial user device and a physical model of the initial user device).

However, the data quality module and/or models therein can be otherwise trained.

In an example, the method can include: using an image sensor, sampling a set of images of a body region of a user; determining a PG dataset based on the set of images; using a trained model, determining a placement of the body region relative to the image sensor based on a set of attributes extracted from the set of images; processing the PG dataset in response to detecting that a set of criteria for the placement of the body region are satisfied; and determining a cardiovascular parameter based on all or apportion of the PG dataset. In a specific example, detecting that the set of criteria for the placement of the body region are satisfied can include: detecting contact between the body region and the image sensor, detecting an acceptable placement of the body region on the image sensor, detecting an acceptable contact pressure between the body region and the image sensor, detecting an acceptable level of body region motion, and/or any other criteria.

In an example, processing the PG dataset can include determining a signal quality for all or a portion of the PG dataset (e.g., using the signal quality model). In a specific example, the method can include: in response to detecting that the signal quality satisfies one or more signal quality criteria, determining a cardiovascular parameter based on the PG dataset; and, optionally, in response to detecting that the signal quality does not satisfy one or more signal quality criteria (e.g., the same or different criteria), guiding the user (e.g., to increase or otherwise adjust a temperature of the body region, to retry the data collection, etc.).

In a specific example, processing the PG dataset can include: segmenting the PG dataset into segments (e.g., corresponding to heart beats); for each of the segments, determining a signal quality for the segment; and determining a subset of the segments associated with a signal quality that satisfies one or more signal quality criteria. A cardiovascular parameter can optionally be determined based on the subset of segments satisfying the criteria (e.g., determining the cardiovascular parameter based on fiducial model(s) fit to the subset of segments). In a specific example, the cardiovascular parameter can be determined in response to detecting that greater than a threshold number of segments (e.g., at least: 5 segments, 10 segments, 12 segments, 15 segments, etc.) are associated with a signal quality that satisfies the signal quality criterion. In another specific example, a user can be guided in response to detecting that less than a threshold number of segments (e.g., the same or a different threshold number of segments) are associated with a signal quality that satisfies one or more signal quality criteria.

In examples, the signal quality criteria can include a signal power criterion, a correlation criterion, a fit criterion, a combination thereof, and/or any other criterion. In a first specific example, the signal quality for a segment can include a signal power metric, wherein the signal quality for the segment satisfies the signal quality criterion when the signal power metric is greater than a threshold. In a second specific example, the signal quality for a segment can include a local correlation metric and/or a global correlation metric (e.g., determined using a second derivative of the segment), wherein the signal quality for the segment satisfies the signal quality criterion when the local correlation metric is greater than a first threshold and/or the global correlation metric is greater than a second threshold. In a third specific example, a fiducial model can be fit to a segment (e.g., fit to the segment, to a first derivative of the segment, to a second derivative of the segment, and/or any other processed or unprocessed PG data), wherein the signal quality for the segment can be determined based on a loss for the fitted fiducial model. In a fourth specific example, a fiducial model can be fit to a segment, wherein the signal quality for the segment can be determined based on fit parameters for the fiducial model and optionally fit parameters for a fiducial model fit to one or more adjacent segments (e.g., the two adjacent segments).

### 6. ILLUSTRATIVE EXAMPLES

The system and/or method can use all or portions of a software design as described in U.S. Provisional Application No. 63/419,189 filed 25 Oct. 2022, which is incorporated in its entirety by this reference.

### Illustrative Example 1

In a first illustrative example, the system and/or method can use all or portions of a software design as described below.

1. Introduction

1.1. Acronyms and Abbreviations

| Acronym or | |
|---|---|
| Abbreviation | Description |
| API | Application Programming Interface |
| App | Application |
| AWS | Amazon Web Services |
| BP | Blood Pressure |
| BPM | Beats Per Minute |
| Cuff | ISO 81060 compliant blood pressure device or via |
| | auscultation |
| FPS | Frames Per Second |
| GPU | Graphics Processing Unit |
| HCP | Healthcare Professional |
| HF | Human Factors |
| HIPAA | Health Insurance Portability and Accountability Act of 1996 |
| HR | Heart Rate (aka Pulse Rate) |
| HTTP | Hypertext Transfer Protocol |
| HTTPS | Hypertext Transfer Protocol Secure |
| Hz | Hertz (e.g., N per second) |
| IFU | Instructions For Use |
| iOS | Apple's iOS Operating System |
| ISO | International Organization for Standardization |
| JSON | JavaScript Object Notation |
| JWT | JSON Web Token |
| MD5 | Message-Digest Algorithm |
| ML | Machine Learning |
| mmHg | Millimeters of Mercury |
| MPS | Metal Performance Shaders |
| OS | Operating System |
| PPG | Photoplethysmogram |
| RGB | Red-Green-Blue |
| S3 | Amazon Simple Storage Service |
| SaMD | Software as a Medical Device |
| SDD | Software Detailed Design |
| SDK | Software Development Kit |
| SSL | Secure Sockets Layer |
| TLS | Transport Layer Security |
| UI | User Interface |
| URL | Uniform Resource Locator |
| UX | User Experience |
| Y′CbCr | Luma (Y) and chroma (Cb for chroma-blue, and Cr for |
| | chroma-red) |

1.2 Definitions

| Term | Definition |
|---|---|
| Accelerometer | Hardware which measures the rate of change of velocity |
| Access Token | An authentication token, such as a JWT token |
| Accumulated window | The period of time over which data is accumulated |
| Accumulator | A mechanism by which data is accumulated, or recorded, |
| | over time |
| Aperture | A variable opening/space through which light passes in |
| | order to reach a camera's sensor |
| App | A software application which can be executed (run) on a |
| | Mobile Device |
| App Store | An app available on iOS which enables users to download |
| | apps, such as those embedding the BP Monitor |
| Application | A programmatic interface containing a set of functions |
| Programming Interface | which allow access to a separate service, system, or |
| | module |
| Bearer Token | A type of access token, typically used during network |
| | requests to authenticate the request |
| Binary Classifier | A classifier which categorizes elements into two groups, |
| | e.g., success/failure |
| Biometrics | The measurement and/or analysis of a person's physical |
| | and/or physiological characteristics |
| Blood pressure | The force of circulating blood on the walls of the arteries. |
| | Blood pressure is taken using two measurements: systolic |
| | (measured when the heart beats, when blood pressure is at |
| | its highest) and diastolic (measured between heart beats, |
| | when blood pressure is at its lowest) |
| Buffer | A temporary data store, typically in memory |
| Calibration | A set of features, derived from a sequence of cuff-based |
| | and camera-based readings, used to subsequently |
| | calculate a blood pressure |
| Calibration procedure | A procedure performed by the BP Monitor SDK which |
| | calculates a calibration |
| Camera-based reading | A measurement taken using the camera on a mobile |
| | device, such as during a calibration procedure or blood |
| | pressure measurement, using the BP Monitor SDK |
| Chroma | A representation of a video's color, often as a red and blue |
| | channel separate from the luma (black-and-white) portion |
| | of a color space |
| Chroma subsampling | A type of compression that reduces the color information in |
| | a signal in favor of luminance data |
| Cloud | A remote server or collection of servers, such as BP Cloud |
| Color depth | The number of bits used to define the color channels (red, |
| | green, blue) for each pixel in an image |
| Crash Report | A collection of data that includes information about the |
| | environment of the software when a crash occurs |
| Cuff-based reading | Blood pressure measurement taken with an ISO 81060 |
| | compliant blood pressure device or via auscultation |
| Device Motion | A measure of how much a device is moving in space (e.g., |
| | acceleration, gravity, yaw, pitch) |
| Diastolic BP | The minimum blood pressure, between two heart beats |
| | when the heart is relaxed and fills with blood |
| Enum | A defined grouping that has a fixed set of related values |
| Exposure | The amount of light which has reached a camera's sensor, |
| | primarily dependent upon aperture, ISO, and shutter speed |
| | settings |
| Finger Detection | An on-device machine learning model which detects if a |
| | person's finger is on the camera lens, as a binary classifier |
| Finger Guidance | An on-device machine learning model which detects where |
| | a person's finger is on the camera lens, providing guidance |
| | for corrections as needed |
| Focal Point | The focus, or area of interest, a camera is set to focus |
| | clearly on |
| Frame Rate | The rate at which a camera's video output is buffered, in |
| | units of frames-per-second (FPS) |
| Graphics Processing | A specialized computing processor designed to accelerate |
| Unit | graphics rendering and transformation |
| Gyroscope | Hardware used for measuring or maintaining orientation |
| | and angular velocity |
| Human Factors | Conditions of how people use technology, involving the |
| | interaction of human abilities, expectations, and limitations, |
| | with a system design |
| Image Integral | An algorithm for quickly and efficiently generating the sum |
| | of values in a rectangular area, specifically across an |
| | image's resolution size |
| iOS | Operating system which runs on Apple smartphones and |
| | Mobile Devices |
| ISO | A camera's sensitivity to light |
| Jail break/Jail broken/ | A mobile device which has had a subset of its OS-level |
| Rooted | security controls broken |
| JSON Web Token | An access token, per RFC 7519, for representing claims |
| | securely between two parties |
| JWT Claim | Pieces of information asserted about a subject encoded |
| | within a JWT, such as their identifier |
| Kernel | A function executed on a GPU, such as for processing |
| | video data into a PPG signal |
| Keychain | An encrypted key-value storage system built into iOS |
| Luma | A representation of a video frame's brightness and |
| | intensity, derived from the achromatic (black and white) |
| | portion of a color space |
| Luminance | A representation of the light intensity of a video frame's |
| | brightness and intensity, derived from the luma portion of a |
| | color space |
| Machine Learning | A methodology of using algorithms and statistical models to |
| | analyze and draw inferences from patterns in data |
| MD5 Hash | A cryptographic function producing a 128-bit hash value of |
| | an input, often used as a checksum to verify the integrity |
| | and immutability of data |
| Metal | An Apple iOS framework for directly accessing a mobile |
| | device's graphics processing unit (GPU) and performing |
| | image processing tasks |
| Mobile Device | A commercially off-the-shelf (COTS) computing platform |
| | that is handheld in nature |
| Notification/Local | An alert banner shown on a user's mobile device, typically |
| Notification | on the mobile device lock screen |
| Parent App | A software application which embeds and executes the BP |
| | Monitor SDK |
| Photoplethysmogram | An optically-obtained plethysmogram that can be used to |
| (PPG) | detect blood volume changes in peripheral circulation |
| Prescription | A mechanism by which users are prescribed use of the BP |
| | Monitor, including of a short code the user types in during |
| | setup |
| Pulse rate/Heart rate | Number of times the heart beats within a certain time |
| | period |
| Resolution | The total number of pixels in a given video frame, typically |
| | given as a width and height |
| BP Cloud | BP Cloud interfaces with the BP Monitor SDK installed on |
| | user Mobile Devices to facilitate blood pressure |
| | measurement sessions and to support other BP Monitor |
| | SDK related functionalities |
| BP Monitor | The collective system of software (inclusive of SDK and BP |
| | Cloud) which enables a PPG to be converted into a blood |
| | pressure measurement |
| BP Monitor SDK | An embedded software package designed to run on user |
| | Mobile Devices that captures a PPG and provides a blood |
| | pressure measurement to the user |
| RSA | An algorithm for public-key cryptography |
| SHA256 | A cryptographic hash function that outputs a value that is |
| | 256 bits long |
| Shutter speed | The length of time a camera's aperture is kept open for, in |
| | order to let light pass through |
| Software Development | A software executable which can be securely embedded |
| Kit (SDK) | into an app and executed |
| Systolic BP | The maximum blood pressure experienced during |
| | contraction of the heart |
| Torch/Flash | The light source for a camera. Torch indicates a |
| | continuously enabled light source, such as for video, |
| | whereas a flash is used temporarily for photos |
| User | The person using the SaMD |
| User Experience | The overall experience of an end user with a device, |
| | product, system, design, or workflow |
| User Interface | A graphical interface through which an end user may |
| | interact with a product or device, often governing the |
| | underlying user experience |
| Video Frame | An individual image frame within a contiguous stream of |
| | video data |
| White balance | An adjustment of the intensities of an image or video's |
| | colors in order to remove unnatural or unwanted colors |
| Xcode | Apple's integrated development environment for macOS, |
| | used to develop software for iOS and mobile devices |
| Y′CbCr | A family of color spaces used in digital video and images, |
| | denoting the luma (Y) and chroma (Cb for chroma-blue, |
| | and Cr for chroma-red) values of the color space |

2. System Overview

2.1 System Components

The BP Monitor can include two subcomponents: Pre-processing: BP Monitor SDK, designed to run on a user's iPhone device and convert video frames into a PPG signal; and Post-processing: BP Cloud, interfaces with mobile SDK to convert a PPG signal into a blood pressure calculation or calibration.

The system is designed to facilitate collection and analysis of PPG data, derived from camera-based video collection with the user's finger placed on the camera, illuminated by the smartphone's torch (light).

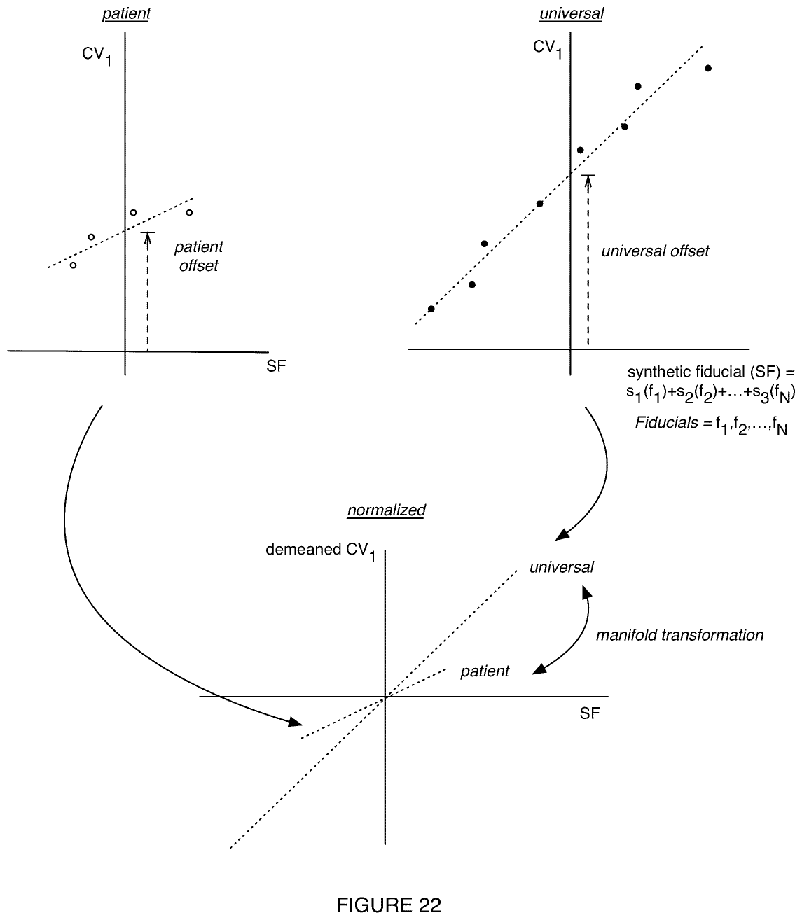

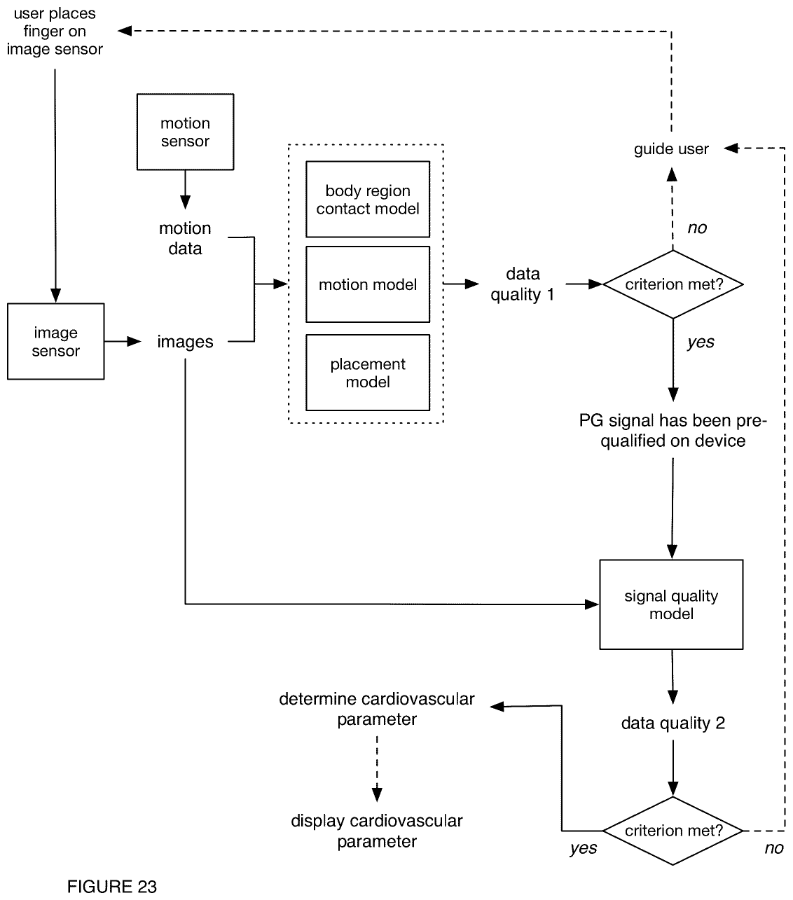

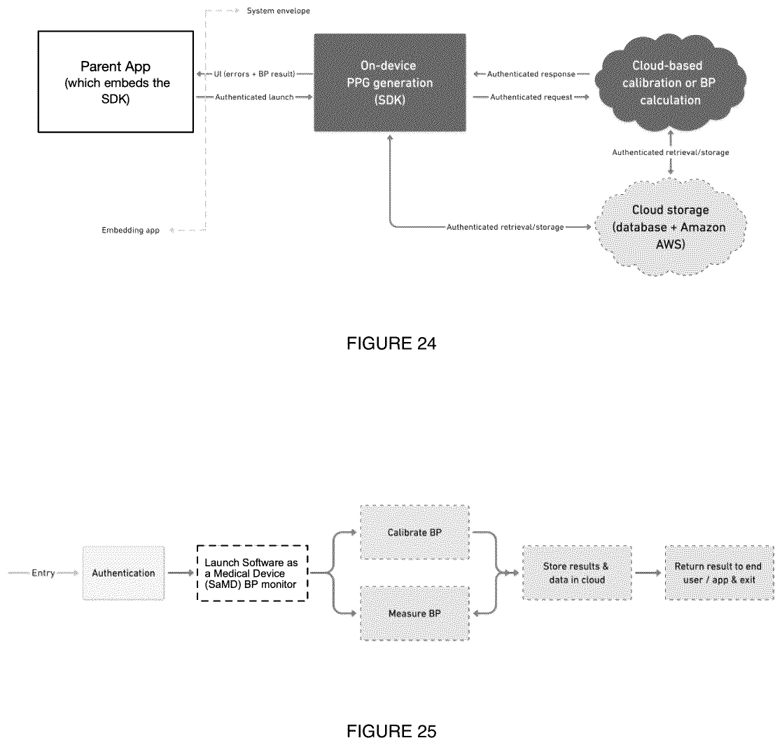

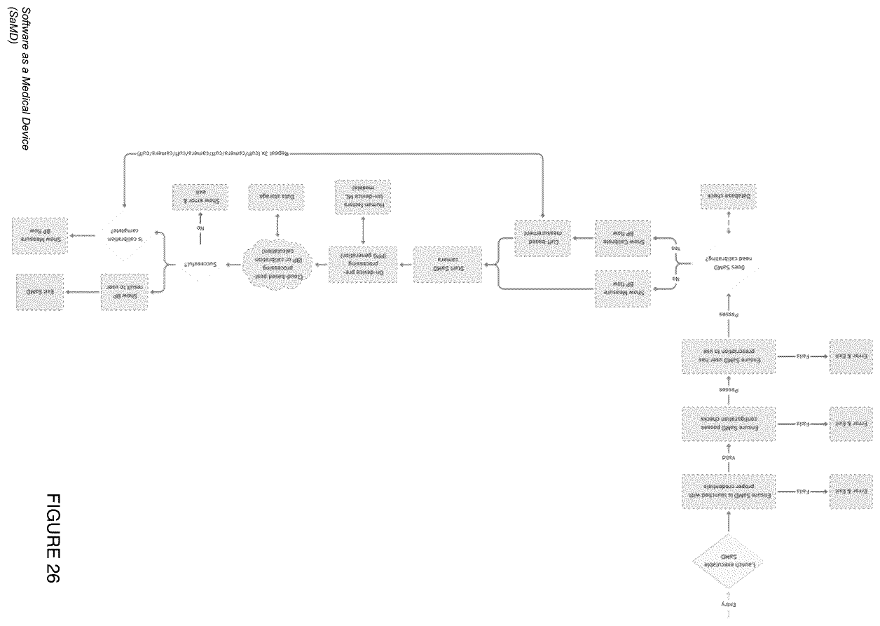

Examples are shown in FIG. 24, FIG. 25, FIG. 26, and FIG. 27.

3. PPG Generation

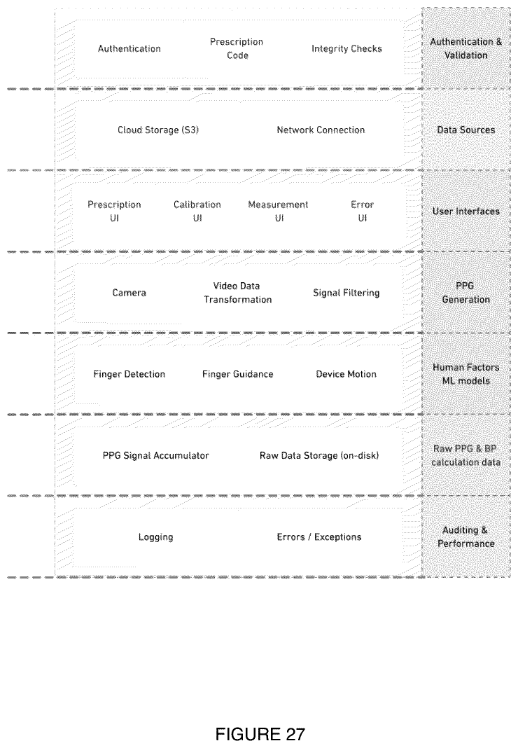

The primary objective of the SDK is to generate a PPG signal of sufficient quality that can be used in either the BP Calibration procedure or BP Calculation. There are controls in place at each step of the generation process to validate on-device quality, in addition to advanced PPG signal quality checks within BP Cloud. An example of a PPG generation flow diagram is shown in FIG. 28.

3.1 Mobile Device's Camera

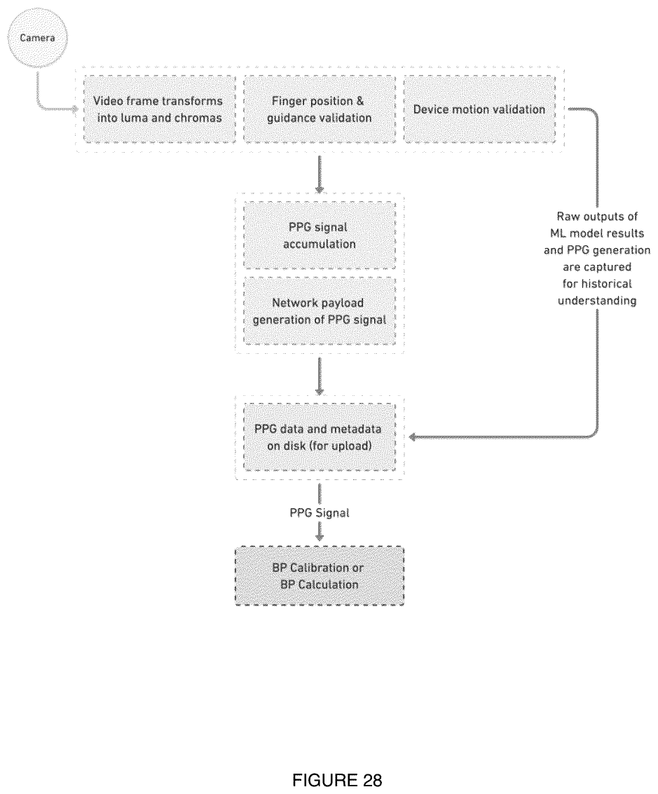

The entry step of the PPG generation step is live, high-speed video capture from the mobile device's digital camera. The camera is configured to generate uncompressed video frames with an emphasis on signal quality and the aperture, shutter speed, light sensitivity (ISO), and white balance values that best enable it. An example is shown in FIG. 29.

3.1.1 Camera Configuration

In an example, the high-level camera configuration steps include: Find required camera lens (ultra-wide angle, rear-facing); Set the output to discard late video frames; Set video orientation to portrait mode; Set video resolution to 1280 pixels in width by 720 pixels in height; Set the pixel format to capture luminance and chroma information across the full operating range of the camera (i.e. a value of kCVPixelFormatType_420YpCbCr8BiPlanarFullRange); Set the frame rate to 120 image frames captured per second; Set camera lens focal point to nearest point and lock the focus (e.g., disable autofocus); Set and lock video output white balance gains to unity (maximum) across all of the color channels (red, green, blue) to ensure data capture without color bias; Set video exposure to 1/120 and light sensitivity (ISO) to the maximum supported ISO value; Delegate video output buffers to a background queue; Create observers for key camera functionality and performance monitoring; Start video capture and turn on torch/flash and set its intensity value to be between 90% and 100% of maximum possible intensity.

The configuration values are implemented with assertions on each to ensure they are properly set.

The rear-facing, ultra-wide angle camera lens is specified to enable a wide viewing angle of the user's finger once placed on the camera lens and offers usability and comfort to the user in terms of hand placement and grip on their mobile device.

The pixel format can determine the color range and output format of the resulting video output; in this case it describes an image with a bi-planar component Y′CbCr, 8-bit color depth, 4:2:0 chroma subsampling, and full-range color (luma=[0,255]chroma=[1,255]). Chroma subsampling can be used to aide with performance.

3.2 Luma and Chroma Features

As was previously stated in the camera section, each video frame has an accompanying image buffer which is decomposed into two planes: luma and chroma. The following section will describe how these planes are transformed from an image buffer into multiple features: 1) Summed overall luminance of each video frame over time: describes a PPG signal; used during BP Calibration and BP Calculation as well as within the Finger Detection module; 2) Summed row-column luminance of each video frame over time: describes brightness of the video frame image for each individual row and column within the frame's resolution size; used within the Finger Guidance module; 3) Summed overall chroma red and blue values of each video frame over time: describes the red and blue color intensities, individually, of the entire video frame image; used within the Finger Detection module.

3.2.1 Luma

Once each video frame is generated by the mobile device camera, the next step in PPG signal generation is to transform the image data from the video frame into the feature required for BP Calibration and BP Calculation: the summed intensity of luma.

3.2.1.1 Summed Overall Luminance of Each Video Frame Over Time

Luminance is of direct importance to PPG signal generation since it denotes the overall brightness of each pixel within the video frame's image; when all luminance values within the entire video frame image are summed, we arrive at the summed luminance intensity for that specific video frame, or point in time, for the PPG signal. Therefore, each summed luminance intensity value—for each video frame-represents a contiguous point within the PPG signal time-series dataset and the overall dataset represents the Summed overall luminance of each video frame over time, also known as a PPG signal. This process is described further in Image Integral below.

Plotted over time (x-axis), with the y-axis being the luminance intensity, a PPG signal is visualized. This is a reflectance PPG signal, where the transmitted light and received light are on same side of the tissue being illuminated. Notably the reflected luminance intensity reduces as the blood pulse flows through the arteries, due to increased density of the pulse.

3.2.1.2 Summed Row-Column Luminance of Each Video Frame Over Time

In addition to the overall luminance intensity, or PPG signal, another useful feature is the individual row-column sums of the luminance intensity for each video frame. This process is described further in Row-Column Image Reduce below. Briefly, luminance intensity information in each row in the image matrix is summed along the columns to generate an array of summed values, called [RowLuminanceIntensitySum]. A similar process is repeated for each column to generate an array of summed intensities, called [RowLuminanceIntensitySum]. These row and column sum arrays are computed for each video frame.

This feature helps describe which portion of the camera lens is potentially covered and uncovered by a user's finger, with exceptionally bright areas potentially indicating light leakage from the torch/flash. The more light leakage, the greater likelihood of a user's finger being off center and the need to encourage the user to recenter their finger placement.

3.2.2 Chroma

Whereas luma is achromatic (black and white), chroma values provide the red and blue color information for a video frame's image and are useful in detecting and guiding a user's finger towards the best placement and pressure on the mobile device camera lens.

3.2.2.1 Summed overall chroma red and blue values of each video frame over time

Similar to a PPG signal, all pixels within each channel of the red/blue chroma plane of a given video frame can be summed to arrive at an overall chroma red/blue intensity for each video frame over time; this summation process is described further in Image Integral below. These features help describe the overall color information for each red or blue channel at a given point in time and are useful alongside luminance within the Finger Detection module to better understand if a user's finger is currently on the camera lens or not. As an example, is the color and intensity change indicative of blood pulsing through a finger over time, or is it more indicative of open air or some other non-compliant or inanimate object?

3.3 GPU Transformations

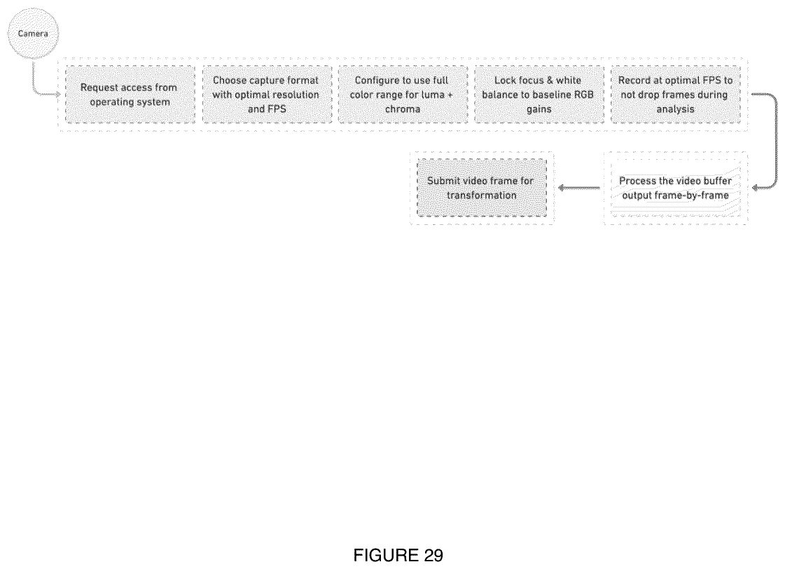

In order to maintain a highly performant PPG signal generation process, the GPU is leveraged to perform image processing in real-time while the mobile device's camera is live streaming raw video output to memory. An example Transformation flow is shown in FIG. 30.

3.3.1 Image Integral

An image integral is the sum of all values in the image frame. In this case, the values being summed are the luminance or chroma red/blue intensities, respectively, in each video frame.

3.3.2 Row-Column Image Reduce

Row-column image reducing functions perform summations of each unique row and column of the image's resolution. For example, the video frames captured by the SDK have a resolution of 1280×720 pixels thus the resulting Row-Column Image Reduce operations will contain an array of 1280 rows [RowIntensitySum] and an array of 720 columns [ColumnIntensitySum] for each video frame.

3.4 Human Factors Controls

This section will describe a systems-level overview of how Human Factors (HF) controls have been implemented on-device, from a risk-based approach.

3.4.1 Human Factors System Overview

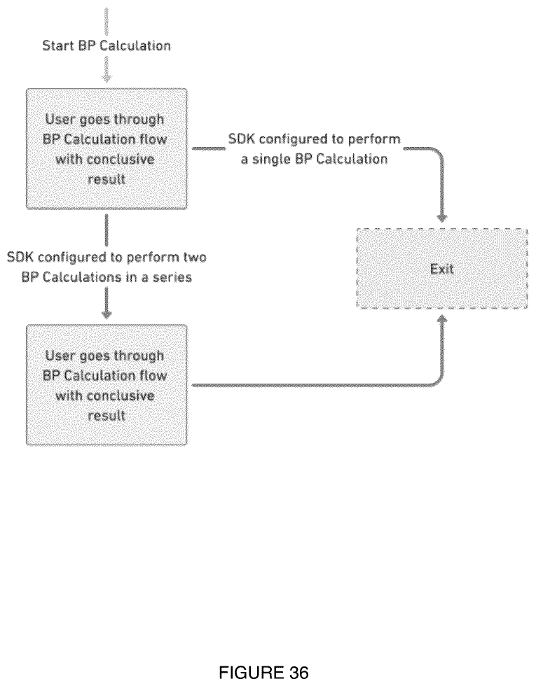

An example of a human factors flow: camera-based reading is shown in FIG. 37.

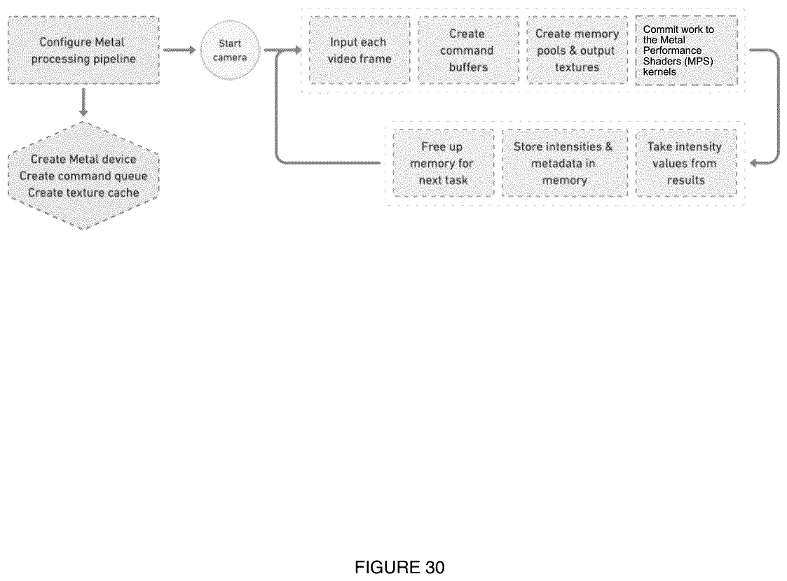

An example of a human factors flow: cuff-based reading is shown in FIG. 31.

3.4.1.1 Placing a Finger on the Camera

As mobile device users are not naturally accustomed to placing their finger on the device's camera lens, it's important to actively guide users in real-time towards proper use of the BP Monitor in multiple ways, beyond them reading the Instructions for Use.

The SDK incorporates UI prompts for proper positioning of the person's body and arm level, and when the camera-based reading begins the user is presented with a live preview of the camera video stream in order to understand which camera to place their finger on. Showing a live video preview results in faster, appropriate finger placement and higher success rates in using the SDK. This is especially true in mobile devices with multiple backward-facing cameras.

Once the user sees the live video preview, they can immediately see which direction the camera is pointing at (e.g., the rear-facing camera is on) and can then quickly align their finger to cover the live video preview while gripping the phone in a very natural position-likely the one they already hold the mobile device with.

Throughout the entirety of the camera-based reading, on-device machine learning models are continuously checking if the user's finger is placed correctly on the lens. In the initial state, the live video preview is shown until the user's finger is initially detected; thereafter, if the user's finger is undetected, the user is shown a resolvable error UI prompt and asked to readjust their finger placement in order to continue the reading. In this way, the user understands when the reading starts and how to correct a finger placement issue if one were to arise. If the user's finger cannot be initially detected for 30 seconds, the SDK will automatically cancel the reading and either allow the user to try again when they're ready, or to cancel the session.

3.4.1.2 Achieving High-Quality PPG Signals

On-device machine learning models are used to pre-qualify the PPG signal in real-time as it's being generated. The On-Device Machine Learning Models section goes into further detail, however of note at a human factors level is that these models help in the following ways: 1) Reset and pause PPG signal accumulation when the ML models have detected undesirable conditions (e.g., device is moving or finger is not placed properly); 2) Automatically restart PPG signal accumulation once the ML models have detected conditions are desirable again; 3) Provide real-time feedback to the user as soon as the SDK detects an issue, better enabling the user to resolve the issue quickly with contextual guidance from the SDK; 4) Allow the SDK to automatically cancel a camera-based reading if the user cannot resolve an issue after 20 seconds of displaying the error (such as the device moving too much for too long from their hands trembling)

3.4.1.3 Providing Feedback for Low Signal Quality

In addition to the on-device machine learning models, the BP Cloud has additional diagnostic checks built-in to help guide the user towards intended use. One of these human factor checks occurs when PPG signals are submitted to BP Cloud for a BP Calibration or BP Calculation, wherein that service will return an error to the SDK if it determines the user potentially has a cold finger due to a low signal quality issue.

3.4.2 Human Factors: Cuff-based Readings

During a calibration procedure, the user is required to input a cuff-based or auscultation-based blood pressure reading using the mobile device keypad. The following human factors checks are integrated to assist with accurate input.

3.4.2.1 Pauses After Occlusive Pressure

As is discussed in the Calibration Procedure section of this document, the SDK ensures the user's arm has time to normalize after occlusive pressure is applied and released following a cuff-based reading. This takes the form of a 60-second countdown timer which prevents the user from continuing with the calibration procedure until the requisite time has elapsed.

3.4.2.2 Range Checks

These range checks will not allow the user to continue to the next screen until they have been corrected: Systolic blood pressure (inclusively between 70 and 200 mmHg); Diastolic blood pressure (inclusively between 45 and 120 mmHg); Pulse rate (inclusively between 20 and 200 beats per minute); Critically high systolic or diastolic blood pressure (greater than or equal to 300 mmHg); Systolic and diastolic blood pressure values appear to be swapped (e.g., user input a diastolic value which was greater in value than the systolic value).

3.4.2.3 User Verification of Manual Cuff-Based Inputs

Additionally, the user is shown a verification UI prompt where they are required to verify the manually input cuff-based blood pressure readings against the source of those values. See Human Factors Flow: Cuff-based Readings for this flow.

This check will allow the user to go back and edit the values if the user finds them to be incorrectly input; additionally, the user can always recalibrate the device at any point using new cuff-based values.

3.4.2.4 Elapsed Time Between Cuff-based Readings

The SDK enforces a maximum allowable user dwell time of ten (1o) minutes between sequential cuff-based and completed PPG readings during the same calibration procedure. If the user exceeds this time interval, they are prompted with an informative error and the calibration procedure is automatically cancelled by the SDK. Rationale for the ten-minute maximum dwell time is discussed in the Calibration Procedure section.

3.5 PPG Signal Accumulation

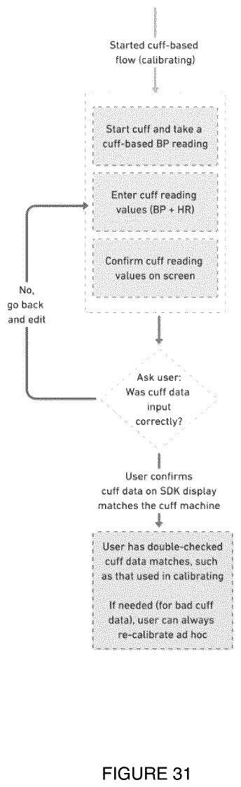

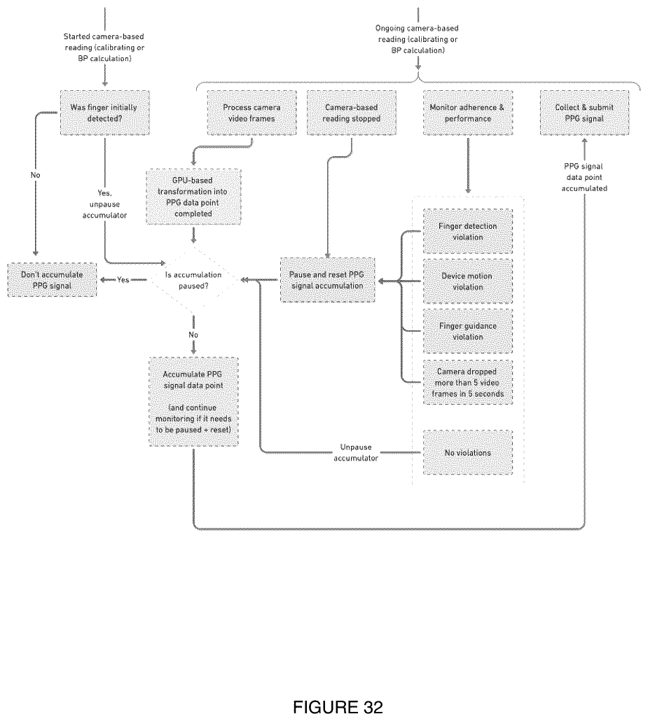

The SDK utilizes an accumulator to capture prequalified, individual PPG data points into memory (i.e., after GPU transformation of a video frame, while not experiencing any human factor violations or exceeding camera frame drop limits). Once the accumulator has captured the requisite data for the camera-based reading scenario (BP Calibration or BP Calculation), it submits the accumulated PPG signal to the BP Cloud for further processing. An example of accumulation start/reset flow is shown in FIG. 32. An example of accumulation collect and submit flow is shown in FIG. 33.

3.5.1 Accumulation Scenarios

There are two PPG signal accumulation scenarios the SDK is configurable for: 1) BP Calibration: 30 second accumulated windows; 2) BP Calculation: 15 second accumulated windows.

When the appropriate amount of PPG data is recorded, the accumulated window is sent via a network request to BP Cloud for further processing. The accumulated window is maintained in memory on the mobile device in case BP Cloud returns an error to the SDK that the PPG signal did not contain enough information to perform the request (e.g., not enough valid heart beats in the accumulated window). If this occurs, the SDK will acquire and append additional data into the existing accumulated window in 15 second increments and re-submit the request to BP Cloud to attempt again. The SDK will submit to BP Cloud up to a maximum of 3 times, incrementally adding to the accumulated window each time, within the same reading session. If at that point BP Cloud still does not have enough information to either calibrate the user or to calculate their BP, the SDK will prompt the user with an error and allow the user to retry the camera-based reading in its entirety.

Of note, the signal accumulator calculates the number of seconds accumulated based upon the video frame metadata itself, e.g., the time delta in seconds between the oldest and newest accumulated video frames where the time is taken from the camera's timestamp for a given video frame

4 User Experiences

As described in Human Factors and other sections, the SDK has implemented a robust set of user interfaces and experiences in order to guide the user towards proper use of the BP Monitor.

4.1 Calibration Procedure

Prior to being able to calculate their blood pressure using the BP Monitor, the user can first calibrate it using an ISO 81060 compliant (e.g., cuff-based) blood pressure monitor or auscultation.

After calibrating the BP Monitor, the user can measure their blood pressure with it for a period of 24 hours, after which time the monitor will prevent the user from taking further blood pressure readings until the monitor is recalibrated.

Procedure

The calibration procedure can include a bracketed series of measurements, with pauses after cuff-based measurements to allow time for the user's arm to normalize after an occlusive pressure was applied. Since a camera-based reading does not occlude the person's blood flow, there is no pause after other than to help instruct the user on the procedure's progress.

In an example, the calibration procedure is as follows: Cuff-based reading; 60-second pause; Camera-based reading; Cuff-based reading; 60-second pause; Camera-based reading; Cuff-based reading; 60-second pause; Camera-based reading; Cuff-based reading.

Completion of this procedure results in a total of 4 cuff-based readings and 3 camera-based readings, all of which are submitted to the BP Cloud where additional checks are performed on the submitted data prior to performing a BP Calibration calculation.

Use of Opposite Arms

After performing a cuff-based reading which applies an occlusive pressure to their arm, the user is instructed to utilize their other arm-which did not receive an occlusive pressure—to perform the subsequent camera-based reading. In the scenario where a user does not follow this instruction, the SDK also enforces a 60-second pause in the calibration procedure after a cuff-based reading to allow the user's arm to normalize after an occlusive pressure is applied and release

Elapsed Time Between Cuff-based Readings

Per ISO/DIS 81060-3 Non-invasive sphygmomanometers—Part 3: Clinical investigation of continuous automated measurement type (Draft), the bracketed assessments used for cuff validation limit the time sensitivity of blood pressure changes using cuffs; i.e., cuffs validated in accordance with ISO 81060-2:2018 Non-invasive sphygmomanometers—Part 2: Clinical investigation of intermittent automated measurement type are sensitive to blood pressure changes beyond 10 minutes because the bracketed assessments used to validate blood pressure cuffs take approximately 10 minutes.

Using this reasoning, that a cuff-based measure is consistent for a maximum of 10 minutes, the SDK enforces an equivalent maximum dwell time of 10 minutes allowed between a cuff-based reading and corresponding camera-based reading. After completing a given cuff-based reading, a countdown timer is started with a set value of 10 minutes. If the countdown timer expires without the user having completed the corresponding camera reading in the series, the SDK will automatically cancel the calibration procedure and prompt the user with an informative error.

Once the user has understood the 10-minute limit, they are able to retry the calibration procedure, or cancel the overall procedure and exit the SDK if they choose to.

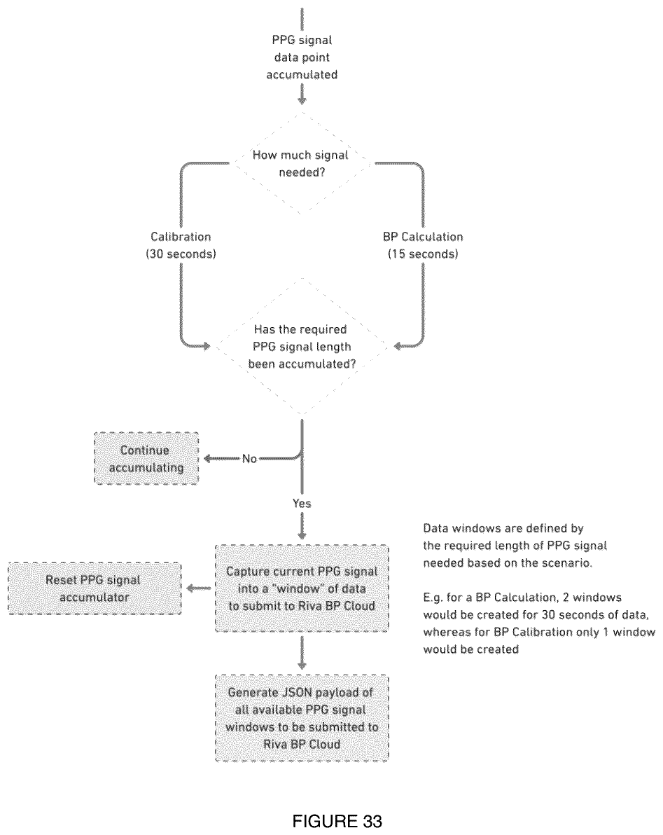

An example of calibration procedure flow is shown in FIG. 34.

4.2 Blood Pressure Calculation

After calibrating the BP Monitor using an ISO 81060 compliant (e.g., cuff-based) blood pressure monitor or auscultation, the user can measure their blood pressure with it for a period of 24 hours, after which time the monitor will prevent the user from taking further blood pressure readings until the monitor is recalibrated.

Per the Indications for Use, the BP Monitor uses the optical signal (photoplethysmogram; PPG) from a fingertip placed on a smartphone torch and camera and calculates changes in blood pressure using the wave shape changes in the PPG.

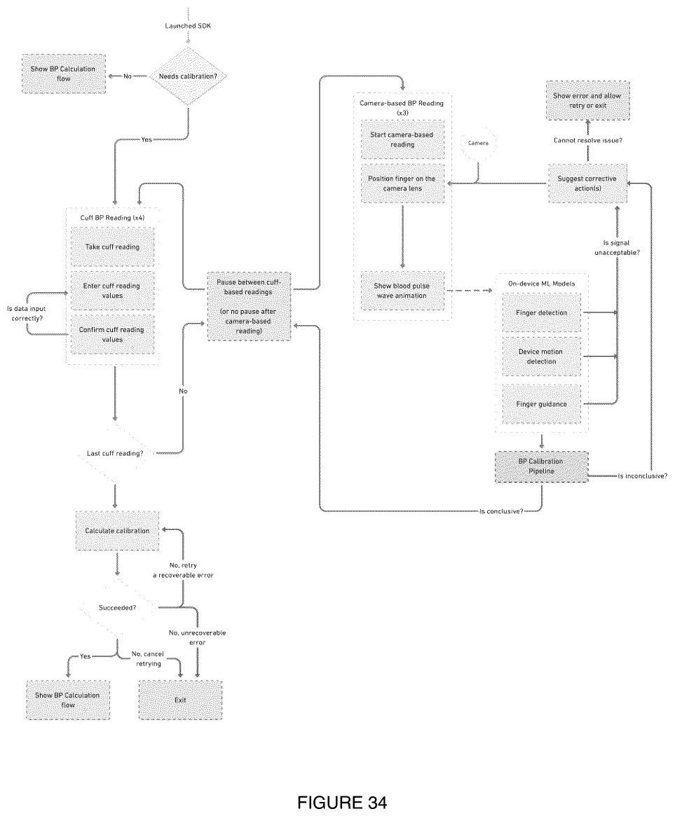

An example of BP calculation flow is shown in FIG. 35.

Pulse Rate

In addition to calculating systolic and diastolic blood pressures, the BP Monitor also calculates the user's pulse rate (colloquially termed as heart rate, HR) and displays that alongside their blood pressure after a conclusive BP Calculation.

Number of BP Calculations in a Series

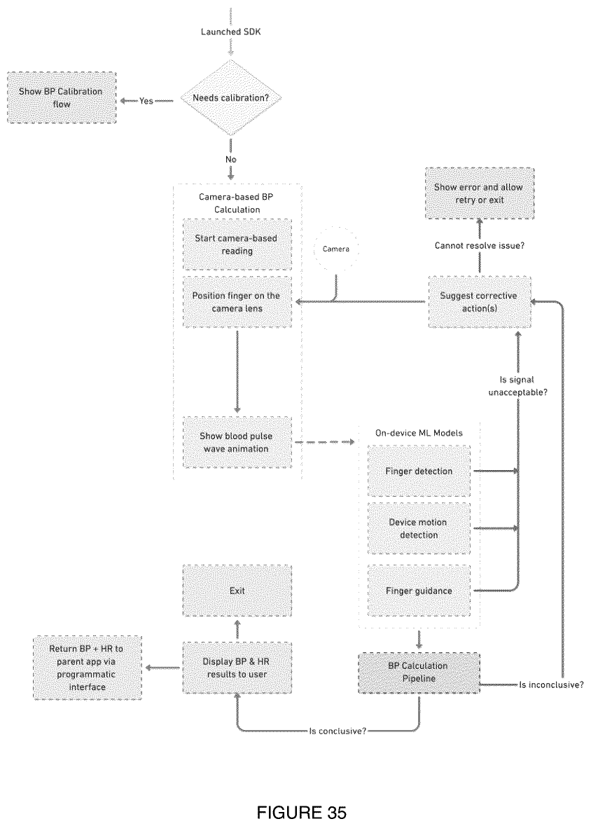

The SDK can be programmatically configured to perform up to two (2) camera-based readings back-to-back within a measurement session, each capturing distinct PPG signals and displaying a distinct result of blood pressure and heart rate. Unless configured otherwise, the SDK defaults into only performing one camera-based reading within a given measurement session. A very short pause may be shown between the readings, just to aid the user in understanding that another camera-based reading will be performed next. An example is shown in FIG. 36.

5 Errors

The BP Monitor has a robust error handling system, with many informative error screens displayable to the user in order to help them best understand what occurred and how to self-correct as many issues as possible.

5.1 Calibration Errors

Elapsed Time Between Cuff-based Readings: As described in the Calibration section of this document, if the user dwells for too long between a completed cuff-based and a completed camera-based reading during a calibration, they are shown an error and can restart the calibration procedure when convenient.

Very High Cuff-based Input Value: As described in the Human Factors section of this document, if the user manually inputs a very high systolic or diastolic value during a cuff-based reading as part of a calibration, they are shown an error.

Could Not Calculate Calibration: If after receiving requisite data from the SDK, the BP Cloud is not able to calculate a calibration from the user, the user is shown an error. If the SDK determines the error is recoverable and can be retried (e.g., no internet connection), it enables the user to retry; if the error is unrecoverable, the SDK will exit after, and the user can perform another calibration procedure at their convenience.

Calibration Expired During Ongoing Camera-based Reading: If the user's calibration expires during an ongoing camera-based reading, the BP Cloud will return an error to the SDK and the SDK will display that error to the user. The user will be required to perform a calibration procedure prior to taking any further camera-based blood pressure readings.

Calibration Expired Upon Launch: If the user launches the SDK with an expired calibration, the SDK will display an error to the user. The user will be required to perform a calibration procedure prior to taking any further camera-based blood pressure readings.

5.2 Blood Pressure Errors

Inconclusive Blood Pressure Calculation: If after receiving requisite PPG data from the SDK, the BP Cloud is not able to calculate a blood pressure for the user (e.g., lack of sufficient heart beats or low signal quality), the SDK will automatically stop the camera-based reading and show an error to the user. The user can retry taking another camera-based blood pressure reading at their convenience.

5.3 Camera-based Reading Errors

Note: this section contains errors that apply generically to camera-based readings which can occur within both BP Calibration and BP Calculation flows.

Cold Finger: If after receiving requisite PPG signal data from the SDK, the BP Cloud may determine the PPG signal quality to be low, possibly from the user's hand and/or finger being cold; if this occurs, the SDK will automatically stop the camera-based reading and the user is shown an error. The user can retry the camera-based reading at their convenience.

SDK Made Inactive by the User: If the SDK is made inactive by the user, such as by backgrounding the Parent App or receiving a callback from the OS that the app was made inactive in other ways (e.g., showing the OS notifications center over-top of the SDK), the SDK will automatically stop the camera-based reading and show an error to the user. The user can retry the camera-based reading at their convenience.

User Received/Answered/Was on Active Phone Call: While monitoring the OS phone call notifications, if the SDK determines the user received/answered/was on an active phone call on the mobile device, the SDK will automatically stop the camera-based reading and show an error to the user. The user can retry the camera-based reading at their convenience.

Camera Access Not Authorized: If the user has not granted authorization for the SDK to access the mobile device camera, the SDK will automatically stop the camera-based reading and show an error to the user. The user can retry the camera-based reading at their convenience after granting access in the mobile device settings.

Camera Interrupted By OS: If the OS interrupts the camera for an unspecified reason (e.g., another app utilizing shared resources), the SDK will automatically stop the camera-based reading and show an error to the user. The user can retry the camera-based reading at their convenience.

Camera Torch Turned Off: If the mobile device's camera torch is turned off by the OS due to elevated system pressure during an ongoing camera-based reading, the SDK will automatically stop the camera-based reading and show an error to the user. The user can retry the camera-based reading at their convenience.

Camera Configuration Errors: If the mobile device camera's configuration cannot be set or maintained, the SDK will automatically stop the camera-based reading and show an error to the user. The user can retry the camera-based reading at their convenience. The following conditions will generate configuration errors: Camera configuration failed; Camera experiences lower-level error; Camera shuts down due to elevated operating system pressure; Torch cannot be enabled or disabled; Torch level decreases below 0.9 out of a maximum of 1.0

5.4 Human Factors Errors

Device Moving Too Much: If the mobile device is moving too much (and the SDK is not currently experiencing other errors), the SDK will show a temporary, resolvable error to the user. Once the device motion is deemed acceptable, the error will be automatically hidden.

Finger Not Initially Detected: After the camera-based reading starts, the SDK will attempt to detect the user's finger for up to 30 seconds. If their finger is not detected after that time has elapsed, the SDK will automatically stop the camera-based reading and show an error to the user. The user can retry the camera-based reading at their convenience.

Finger Not Detected (After Initial Detection): After the camera-based reading starts, the SDK will attempt to detect the user's finger. If their finger is initially detected and starts accumulating a PPG signal, but then the finger is no longer detected thereafter (such as the user removing their finger from the camera lens), the SDK will show a temporary, resolvable error to the user. Once the finger is detected, the error will be automatically hidden.

Resolvable Error Is Not Resolved After 20 Seconds: After the camera-based reading starts, the SDK could display resolvable errors, for example the device's motion is unacceptable or finger is not detected. Once a resolvable error is shown, the SDK will pause and purge signal accumulation, and give the user 20 seconds to resolve the error. If the user does not resolve the error within the allotted time, the SDK will automatically stop the camera-based reading and show an error to the user. If the user resolves the error within the allotted time, it will resume the camera-based reading as long as no other resolvable errors have been enqueued.

6 On-Device Machine Learning Models

6.1 Introduction

The on-device Machine Learning (ML) models assist in the proper use of the SDK by the user and help to mitigate Human Factors (HF) risks while improving the user experience. The goal of the collective set of models is to ensure minimal device motion (Device Motion model) and the proper finger placement (Finger Detection and Guidance models) in order to capture a high-quality PPG signal from the user prior to making a network call to the BP Cloud service for BP Calibration or BP Calculation.

6.2 Device Motion

6.2.1 Purpose

The purpose of the Device Motion model is to flag improper device and/or user motion that would lead to an incorrect or suboptimal PPG capture by the user. Classification decisions from this model are used to alter the user experience flow in the BP Monitor SDK by notifying the user so they can adjust their body position and/or device motion to complete an accurate PPG capture.

6.2.2 User Experience

During a BP Calibration or BP Calculation process the SDK makes use of on-device motion sensors to measure the motion of the device during the BP Calibration/BP Calculation process. (See Technical—Inputs for more details).

Motion sensor data is sampled in 2-second windows and at the end of each window the on-device Device Motion Model is called to classify the aggregated data. There are 2 categories of motion in which the ML model serves as a binary classifier: correct motion, and incorrect motion. (See sections below for detailed description of each). If the Device Motion ML model cannot be instantiated, an error is displayed, and the user is prevented from further use during that SDK session.

Over the course of a BP Calibration/BP Calculation as the SDK accumulates a PPG signal, there are numerous device motion windows of motion sensor data that are captured and classified. The classification window for motion data is less than the overall signal accumulated window for user PPG data in order to proactively warn the user of motion that may affect the quality of the acquired PPG signal.

6.2.2.1 Correct Motion

The range and speed of correct device motion by the user during a PPG capture can be empirically defined through bench testing of the model.

The flow of a correct motion measurement can include:

1) The user places their finger on the proper mobile device camera to start the PPG capture.

2) The user remains seated and still while minimizing device/arm/hand/body movements during the measurement period. The user may perform one or more of the following minor movements that would not affect the accuracy of the PPG capture: 1) Device Orientation: Alternative wrist positions; Device Movement: Slowly rotate/adjust wrist, Slight forearm movement/adjustment (up/down), Slight bounce, Slight movement due to breathing and/or talking/yelling

3) Once the analysis has completed successfully the user is shown a success screen with more information.

Correct classifications from the Device Motion model for the course of the measurement will result in an uninterrupted measurement flow without any additional notifications to the user.

6.2.2.2 Incorrect Motion

The range and speed of incorrect device motion by the user during a PPG capture can be empirically defined through bench testing of the model.

The flow of an incorrect motion measurement can include:
1) The user places their finger on the proper mobile device camera to start the PPG capture (See Finger Detection and Finger Guidance sections for more details)
2) The user does not remain seated and/or still and moves their device/arm/hand/body during the measurement period beyond a reasonable amount. The user may perform one or more of the following prohibited movements that would affect the accuracy of the PPG capture: Shaking the device, Rolling/rotating the device, Tapping the device, Lifting finger on/off the camera, Swinging arm, Raising/lowering arm, Bouncing arm/hand, Walking, Running, Squatting, Spinning, Jumping, Going up/down stairs, Getting up/sitting down, Shaking.
3) The analysis is interrupted and the user is notified of their incorrect motion behavior. They are reminded to remain seated and still and the measurement starts over again.

6.2.2.3 UI/UX

Incorrect classification from the Device Motion model for any measurement window will result in an interruption to the measurement process, and the following high-level actions by the SDK: Reset the measurement PPG accumulator; Notify the user to remain still.

6.2.3 Technical Specification

6.2.3.1 Input

The movement of the user/device is captured via a number of on-device motion sensors sampled at 60 Hz (sample/second) and classified over an accumulated 2-second window of measurements for a total of 120 samples per classification. There are 12 motion sensor input channels for 4 different categories of motion sensing: Gravity (X,Y,Z) (e.g., Float32 with a 2-dimensional shape of [120×3]); Acceleration (X,Y,Z) (e.g., Float32 with a 2-dimensional shape of [120×3]); Rotation (X,Y,Z) (e.g., Float32 with a 2-dimensional shape of [120×3]); Attitude (Pitch, Roll, Yaw) (e.g., Float32 with a 2-dimensional shape of [120×3]).

6.2.3.2 Output

The weight for each enum case will be given as a percentage, with the overall weight of all enum values for a given prediction adding up to 1.0. The position with the maximum weight shall be taken as the prediction. For example, [0.25, 75] is considered a Correct Motion prediction with 75% confidence.

The output of the model is a motion decision vector with the following one-hot encoding: Correct—Measurement process can proceed with no motion objection (e.g., [0,1]—Correct Motion); Incorrect—Measurement process should be interrupted with a motion objection (e.g., [1, 0]—Incorrect Motion).

6.3 Multi-modal Finger Prediction

The following sections will describe Finger Detection and Finger Guidance in more detail, however the SDK can utilize the combined outputs of both of those modules to arrive at an overall prediction for the same 2-second window of data.

In a specific example, in order for a finger to be determined acceptable on the mobile device camera lens, the following combined outputs must be true (e.g., an AND relationship): Finger Detection result of Finger Detected; Finger Guidance result of Ideal Placement.

6.4 Finger Detection

6.4.1 Purpose

The purpose of the Finger Detection model is to flag if the user has proper finger placement on the mobile device's camera in order to complete an accurate PPG capture. Classification decisions from this model are used to alter the user experience flow in the BP Monitor SDK by notifying the user so they can adjust their posture and/or finger position to complete an accurate PPG capture.

6.4.2 User Experience

During a BP Calibration or BP Calculation process the SDK makes use of the mobile camera to measure the position of the user's finger during the BP Calibration/BP Calculation process. (See Technical—Inputs for more details).

Camera data is sampled in 2-second windows and at the end of each window the on-device Finger Detection Model is called to classify the aggregated data. There are 2 categories of finger placement in which the ML model serves as a binary classifier: correct placement (aka finger detected), and incorrect placement (aka finger not detected). (See sections below for detailed description of each).

If the finger detection ML model cannot be instantiated, an error is displayed and the user is prevented from further use during that SDK session.

Over the course of a BP Calibration/BP Calculation as the SDK accumulates a PPG signal, there are numerous finger detection windows of camera data that are captured and classified. The classification window for camera data is less than the overall signal accumulated window for user PPG data in order to proactively warn the user that a finger is not properly detected and that it is preventing the acquisition of the measurement PPG signal.

6.4.2.1 Correct Placement

The position and pressure for correct finger placement by the user during a PPG capture has been empirically defined through bench testing of the model.

The flow of a finger placement measurement can include: 1) The user is instructed to place their finger on the proper mobile device camera to start the PPG capture. 2) The user remains seated and still while minimizing device/arm/hand/body movements during the measurement period. 3) The user attempts to take an ideal PPG capture with the following combination of allowed finger placement and environmental variations: Finger Orientation (grip dependent), angled with phone (0, 45, 90, 135, 180, 225, 270, 315 degrees); Pressure; Ideal finger pressure (approx. weight of phone)

Once the analysis has completed successfully the user is shown a success screen with more information.

Correct classifications from the Finger Detection model for the course of the measurement will result in an uninterrupted measurement flow without any additional notifications to the user.

6.4.2.2 Incorrect Placement

The position and pressure of incorrect finger placement by the user during a PPG capture has been empirically defined through bench testing of the model.

The flow of an incorrect finger placement measurement can include: 1) The user is instructed to place their finger on the proper mobile device camera to start the PPG capture. 2) The user remains seated and still while minimizing device/arm/hand/body movements during the measurement period. 3) The user attempts to take an ideal PPG capture with the following prohibited finger placement and environmental variations:

With Finger: a) Pressure—Too soft (hover), Too hard (squish); b) Movement: Tapping (finger on-off camera); c) Alignment—Askew from covering center camera (i.e., too far left/right/top/bottom including diagonal); d) Obstruction—foreign material in-between: Band-Aid, Paper, Clothing/fabric

Without Finger: a) Other Body Parts (head, fingernail, etc.); b) Foreign Material (e.g., colored paper, table, carpet, etc.) static and with movement (e.g., trained once for all people since it is not dependent on individual physiology); c) Movement —Tapping camera with finger without phone movement (to mimic the appearance of heart beats in terms of light intensity changes) d) Lighting—Constant exposure to lighting conditions.

4) The analysis is interrupted and the user is notified their finger has not been detected in the proper orientation to record an accurate measurement. The Finger Guidance model can also be used to guide how the user should adjust their finger position to restart the measurement process.

6.4.2.3 UI/UX

Incorrect classification from the Finger Detection model for any measurement window will result in an interruption to the measurement process, as shown, and the following high-level actions by the SDK: Reset the measurement PPG accumulator; Notify the user to check their finger placement on the camera.

6.4.3 Technical Specification

6.4.3.1 Input

A stream of video frames is captured from the mobile device's camera-using a set of verified device-specific camera settings (resolution, frame rate, ISO, exposure, etc.), as reported in the Mobile Device's Camera section-over an accumulated 2-second window of measurements at 120 frames-per-second for a total of 240 samples per classification. There are 3 video input channels: Luminance Intensity—The sum total luminance of a video frame's pixels (e.g., Float32 with a 2-dimensional shape of [240×1]); Chroma Red Intensity—The sum total red chroma portion of a video frame's pixels. (e.g., Float32 with a 2-dimensional shape of [240×1]); Chroma Blue Intensity—The sum total blue chroma portion of a video's frame pixels (e.g., Float32 with a 2-dimensional shape of [240×1]);

6.4.3.2 Output

The weight for each enum case will be given as a percentage, with the overall weight of all enum values for a given prediction adding up to 1.0. The position with the maximum weight shall be taken as the prediction. For example, [0.25, 0.75] is considered a Finger Detected prediction with 75% confidence.

The output of the model is a finger-detection decision vector with the following one-hot encoding: Correct—Measurement process can proceed with a finger properly detected on the camera (e.g., [0,1]—Finger Detected); Incorrect—Measurement process should be interrupted with a finger not detected objection (e.g., [1,0]—Finger Not Detected).

6.5 Finger Guidance

6.5.1 Purpose

The purpose of the Finger Guidance model is to flag if the user has proper finger placement on the mobile device's camera in order to complete an accurate PPG capture. Classification decisions from this model are used to alter the user experience flow in the BP Monitor SDK by notifying the user so they can adjust their posture and/or finger position to complete an accurate PPG capture.

6.5.2 User Experience

During a BP Calibration or BP Calculation process the SDK makes use of the mobile device camera to measure the position of the user's finger during the BP Calibration/BP Calculation process. (See Technical—Inputs for more details).

Measurement camera data is sampled in 2-second windows and at the end of each window the on-device Finger Guidance model is called to classify the aggregated data. There are 2 categories of finger placement in which the ML model serves as a binary classifier: correct placement (aka finger detected), and incorrect placement (aka finger not detected). (See sections below for detailed description of each). If the finger guidance ML model cannot be instantiated, an error is displayed, and the user is prevented from further use during that SDK session.

Over the course of a BP Calibration/BP Calculation as the SDK accumulates a PPG signal, there are numerous finger detection windows of camera data that are captured and classified. The classification window for camera data is less than the overall signal accumulated window for user PPG data in order to proactively warn the user that a finger is not properly detected and that it is preventing the acquisition of the measurement PPG signal.

6.5.2.1 Correct Placement

The position and pressure for correct finger placement by the user during a PPG capture has been empirically defined through bench testing of the model.

Correct classifications from the Finger Guidance model for the course of the measurement will result in an uninterrupted measurement flow without any additional notifications to the user.

The flow of a finger placement measurement can include: The user is instructed to place their finger on the proper mobile device camera to start the PPG capture; The user remains seated and still while minimizing device/arm/hand/body movements during the measurement period; The user attempts to take an ideal PPG capture with finger placement and environmental variations as previously outlined in Finger Detection and Device Motion sections; Finger and measurement signal is properly detected and PPG capture begins; Once the analysis has completed successfully the user is shown a success screen with more information.

6.5.2.1 Incorrect Placement

The position and pressure of incorrect finger placement by the user during a PPG capture has been empirically defined through bench testing of the model.

The flow of an incorrect finger placement measurement can include: 1) The user is instructed to place their finger on the proper mobile device camera to start the PPG capture. 2) The user remains seated and still while minimizing device/arm/hand/body movements during the measurement period. 3) The user attempts to take an ideal PPG capture with one of the following non-ideal finger placements:

a) Finger. Placement: (Slide Up) Too far below camera; (Slide Down) Too far above camera; (Slide Left) Too far to the right of the camera (with the device's front facing the person); (Slide Right) Too far to the left of the camera (with the device's front facing the person); Movement—not enough device movement for Device Motion model to detect (e.g., Tapping).

b) No Finger. Open air (with and without movement); Variety of surfaces.

4) The analysis is interrupted, and the user is notified their finger has not been detected in the proper orientation to record an accurate measurement. The user is offered guidance on how to adjust their current measurement finger position and a new measurement is started.

6.5.2.3 UI/UX

Incorrect classification from the Finger Guidance model for any measurement window will result in an interruption to the measurement process, as shown, and the following high-level actions by the SDK: Reset the measurement PPG accumulator; Notify the user to check their finger placement on the camera.

6.5.3 Technical Specification

6.5.3.1 Input

The finger position of the user is captured via an unfiltered stream of video frames captured from the mobile device's camera-using a set of verified device-specific camera settings (resolution, frame rate, ISO, exposure, etc.), as reported in the Mobile Device's Camera section-over an accumulated 2-second window of measurements at 120 frames-per-second for a total of 240 samples per classification. There are 2 video input channels, recorded with the device camera in a portrait orientation: Row Luminance Intensity—sum over each row of a video frame's pixels, representing the height of the video frame image buffer (e.g., Float32 with a 2-dimensional shape of [240×1280]); Column Luminance Intensity—sum over each column of a video frame's pixels, representing the width of the video frame image buffer (e.g., Float32 with a 2-dimensional shape of [240×720]).

6.5.3.2 Output

The one-hot output of the model is a finger-guidance decision vector with the following encoding and descriptive guidance for the user: Correct—Measurement process can proceed with a finger properly detected on the camera (e.g., [1,0,0,0,0,0,0,0]—Ideal Placement—No Guidance); Incorrect—Measurement process should be interrupted and the user offered guidance to adjust their finger placement and restart measurement (e.g., [0,1,0,0,0,0,0,0]—Decrease Finger Pressure—Finger is on camera but with too much pressure; [0,0,1,0,0,0,0,0]—Increase Finger Pressure—Finger is hovering over camera without enough pressure; [0,0,0,1,0,0,0,0]-Shift Finger Up—Finger is not centered (top of lens exposed); [0,0,0,0,1,0,0,0]—Shift Finger Down—Finger is not centered (bottom of lens exposed); [0,0,0,0,0,1,0,0]—Shift Finger Left—Finger is not centered (left-side of lens exposed); [0,0,0,0,0,0,1,0]—Shift Finger Right—Finger is not centered (right-side of lens exposed); [0,0,0,0,0,0,0,1]—Stop Moving Finger—Finger is Sliding/Rolling (Up/Down/Left/Right) or tapping on camera).

Despite the multi-class output of this model, the Finger Guidance model can be used as a binary classifier with the output No Guidance (Ideal Placement) as the Correct placement indicator and Stop Moving Finger as the Incorrect placement indicator.

The weight for each enum case will be given as a percentage, with the overall weight of all enum values for a given prediction adding up to 1.0. The position with the maximum weight shall be taken as the prediction. For example, [0.75, 0, 0, 0.20, 0, 0, 0.05, 0] is considered an Ideal Placement prediction with 75% confidence.

### Illustrative Example 2

In a second illustrative example, the system and/or method can use all or portions of a software design as described below.

1. Introduction

Acronyms and Abbreviations

| Acronym or | |
|---|---|
| Abbreviation | Description |
| ACL | Access Control List |
| AES | Advanced Encryption Standard |
| API | Application Programming Interface |
| AWS | Amazon Web Services |
| BP | Blood Pressure |
| BPM | Beats Per Minute |
| BPW | Blood Pulse Waveform |
| CIDR | Classless Interdomain Routing |
| CVE | Common Vulnerabilities and Exposures |
| DBP | Diastolic Blood Pressure |
| DNS | Domain Name Server |
| EC2 | Elastic Compute Cloud |
| ECOD | Empirical-Cumulative-distribution-based Outlier |
| | Detection |
| GHA | GitHub Actions |
| HR | Heart Rate |
| HTTP | Hypertext Transport Protocol |
| HTTPS/TLS | Hypertext Transport Protocol Secure over Transport Layer |
| | Security |
| IAM | Identity and Access Management |
| IIR | Infinite Input Response |
| IP | Internet Protocol |
| JSON | JavaScript Object Notation |
| JWT | JSON Web Token |
| KMS | Key Management Service |
| MA | Moving Average |
| MDM | Mobile Device Management |
| MFA | Multi-Factor Authentication |
| ML | Machine Learning |
| NAT | Network Address Translation |
| OIDC | OpenID Connect |
| PHI | Protected/Personal Health Information |
| PII | Personal Identifiable Information |
| PP | Pulse Pressure |
| PPG | Photoplethysmogram |
| QC | Quality Check |
| RDS | Relational Database Service |
| REST | Representational State Transfer |
| S3 | Simple Storage Service |
| SaaS | Software as a Service |
| SaMD | Software as a Medical Device |
| SAML | Secure Assertion Markup Language |
| SBP | Systolic Blood Pressure |
| SDK | Software Development Kit |
| SIEM | Security Information and Event Management |
| S-G | Savitzky-Golay |
| SMS | Short Message Service |
| SSH | Secure Shell |
| SSL | Secure Socket Layer |
| SSO | Single Sign-On |
| URL | Uniform Resource Locator |
| VPC | Virtual Private Cloud |
| VPN | Virtual Private Network |
| WAF | Web Application Firewall |
| YAML | Yet Another Markup Language (File Format) |

### Definitions

| Term | Definition |
|---|---|
| 3rd Party Admin | The role assigned to a 3rd Party Developer when |
| | administering their users and prescriptions. |
| 3rd Party API | A backend service the 3rd Party Developer can implement |
| | for administration and authentication. It interfaces with |
| | BP Cloud. |
| 3rd Party Developer | A software developer who will embed BP Monitoritor into |
| | their mobile device app. |
| Access Token | An authentication token, such as a JWT token. |
| Admin JWT | An access token which is created for a 3rd party Developer |
| | or Application via the exchange of an admin client |
| | identifier and secret. |
| Autho | An authentication and identity verification software |
| | provider. |
| Autho Actions | Autho lambda functions used to customize capabilities |
| | executed during the Autho authorization process. |
| Autho API | API used to access Autho's identity functionality and |
| | protocols. |
| Autho Application | An entity domain object which provides authentication |
| | and authorization configuration values for BP Cloud |
| | Applications and Customer Tenants segmentations. |
| AWS API Gateway (also | An AWS service that accepts API calls and routes them to |
| referred to as API Gateway) | the backend services. |
| AWS Availability Zone (also | A discrete AWS datacenter with redundant power, |
| referred to as Availability | networking, and connectivity within an AWS region. |
| Zone) | |
| AWS CloudTrail (also | An AWS service that monitors and records account activity |
| referred to as CloudTrail) | across the AWS infrastructure. |
| AWS CloudWatch (also | An AWS service that collects monitoring and operational |
| referred to as CloudWatch) | data in the form of logs, metrics, and events. |
| AWS Config | An AWS service that enables the assessment, audit, and |
| | evaluation of the configurations of AWS resources. |
| AWS EKS (also referred to as | An AWS service that provides a managed container service |
| EKS) | and runs Kubernetes applications. |
| AWS Fleet Manager | A subcomponent of AWS Systems Manager that provides |
| | centralized server management processes. |
| AWS GuardDuty (also | An AWS service that continuously monitors all AWS |
| referred to as GuardDuty) | accounts and workloads for malicious activity and delivers |
| | detailed security findings for visibility and remediation. |
| AWS IAM (also referred to as | An AWS service that provides identity and access control |
| IAM) | mechanisms for AWS resources. |
| AWS Lambda (also referred | An AWS service that provides a serverless event driven |
| to as Lambda) | compute platform. |
| AWS Management Account | An AWS account that is used to create and manage an |
| (also referred to as | AWS Organization and the Organization's AWS Member |
| Management Account) | Accounts. |
| AWS Organization | An AWS service that enables the central management and |
| | governance of the entire AWS infrastructure and accounts. |
| AWS Patch Manager | A subcomponent of AWS Systems Manager that automates |
| | the process of scanning and patching compute instances. |
| AWS Private Link (also | An AWS infrastructure component that provides a secure |
| referred to as Private Link) | VPC network connection for AWS services such as API |
| | Gateway. |
| BP-API | A backend service-part of BP Cloud which interfaces with |
| | the BP Monitoritor SDK-which performs specific |
| | functions like authentication, prescription code |
| | verification, etc. and is an interface to other backend |
| | services and database(s). |
| Customer Tenant | A logical separation used to isolate data between different |
| | Customers. |
| OAuth2 | An industry-standard protocol for authorization. |
| On-Call | The ability to be contacted in order to provide a |
| | professional service if necessary. |
| OpenID Connect (also | An open authentication protocol that works on top of |
| referred to as OIDC) | oauth 2.0 framework. |
| PagerDuty | An incident response, management, and resolution |
| | platform for information technology. Provides On-Call |
| | functionality. |
| Parent App | A software application which embeds and executes the BP |
| | Monitoritor SDK. |
| Photoplethysmogram (PPG) | An optically obtained plethysmogram that can be used to |
| | detect blood volume changes in peripheral circulation. |
| Plethysmogram | A measurement of changes in parts of the body. |
| Pod | The smallest execution unit in Kubernetes that contains |
| | one or more applications. |
| Pod Service Account | A permissions configuration for a Pod that provides the |
| | processes with an identity. Also used for Pod |
| | authentication purposes. |
| Postgres (also referred to as | An AWS service that provides managed instances of |
| RDS Postgres) | Postgres, a SQL database used by BP Cloud. |
| BP Cloud | BP Cloud interfaces with the BP Monitoritor SDK installed |
| | on user Mobile Devices to facilitate blood pressure |
| | measurement sessions and to support other BP |
| | Monitoritor SDK related functionalities. |
| BP Monitor | The collective system of software (inclusive of SDK and BP |
| | Cloud) which enables a PPG to be converted into a blood |
| | pressure measurement. |
| BP Monitor SDK (also | An embedded software package designed to run on user |
| referred to as “the SDK”) | Mobile Devices that captures a PPG and provides a blood |
| | pressure measurement to the user. |
| Root Certificate Authority | Primary certificate authority in a certificate authority chain |
| (also referred to as Root CA) | of trust. |
| SDK User | A user of a Parent App, which embeds the SDK. |
| SDK User JWT | The authentication token used to identify SDK Users. |

2.1 System Components

The BP Monitor can optionally include two subcomponents: Pre-processing: BP Monitor SDK, designed to run on a user's iPhone device and convert video frames into a PPG signal; Post-processing: BP Cloud, interfaces with the SDK to create a blood pressure calculation or calibration from the PPG signal.

2.2 Use Cases

BP Cloud, the primary focus of this document, is a collection of backend services to support the calibration, calculation, and collection of PPG signals. This section describes the Use Cases that BP Cloud implements.

The Parent App is a mobile application managed by the 3rd Party Developer, (“The Customer”), which can integrate with BP Monitor SDK to take BP readings. The SDK is embedded as part of the Parent App.

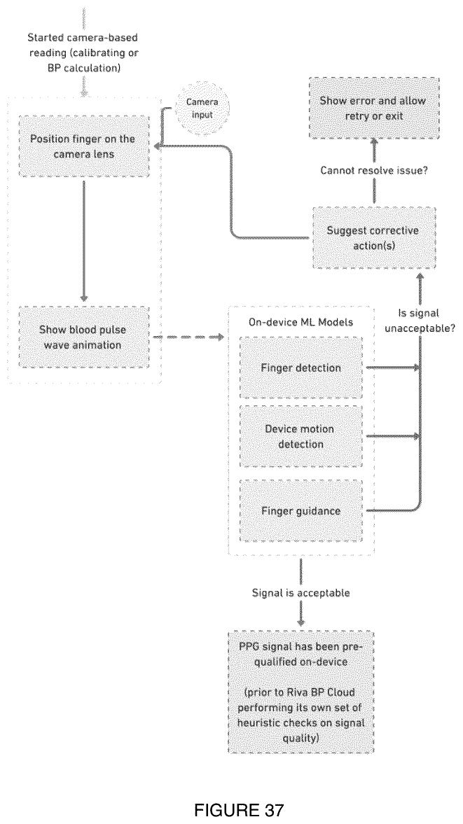

An example flow is shown in FIG. 38.

3 System Architecture

3.1 Calculation and Calibration Lambdas

There are two Python-based endpoints hosted on AWS's event-driven, serverless compute Lambda platform and deployed with other supporting services that are used by the SDK to calibrate and calculate user's BP.

Each endpoint shares several common components that are documented followed by more endpoint-specific details for the BP calibration endpoint and the BP calculation endpoint.

3-1-1 BP Calibration Lambda

The purpose of BP Calibration process is to establish a calibration for the user's systolic (SBP) and diastolic (SBP) blood pressures from which changes can be calculated with the BP Calculation process. On startup the mobile client determines if a valid BP calibration exists for the user. If there is a valid and unexpired calibration for the user, they are allowed to take a camera-based BP reading. If not, the mobile client notifies the user and guides them through a calibration flow to establish a valid and unexpired calibration.

BP Calibration involves taking a series of bracketed measurements with the SDK, specifically: 4 reference (cuff-based) measurements entered by the user and 3 camera-based measurements already collected by the SDK using the BP Calculation Lambda in calibration mode.

After these measurements are taken and validated the mobile client calls the BP Calibration endpoint via its RESTful API to initiate the calibration process.

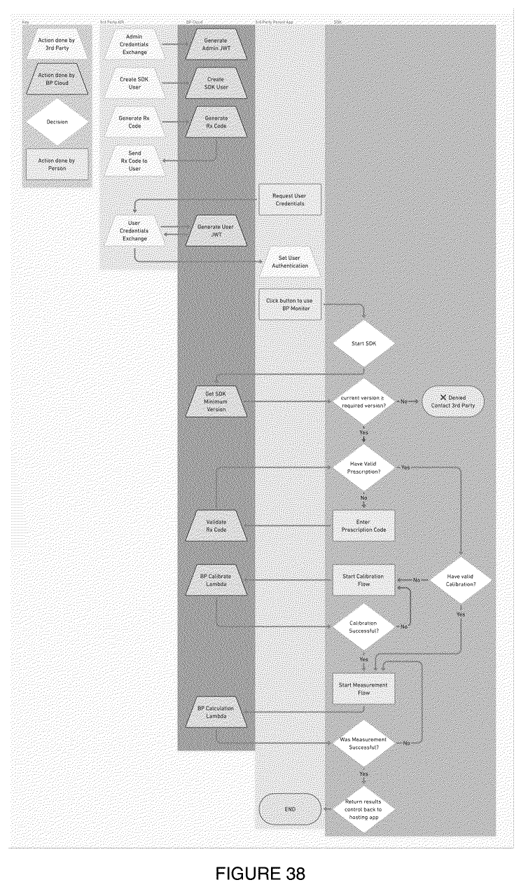

Example BP Calibration Lambda components are shown in FIG. 39.

3.1.1.1 BP Calibration Process

Upon routing of a BP Calibration endpoint request the BP Calibration process begins and can include the following sequential processing steps as detailed in the subsections that follow:
1. List cuff and camera measurements from the bracketed assessment from BP-API for the requested calibrationReadingId.
2. Load camera measurement JSON PPG payload files from S3.
3. Check the cuff measurements with:
a. Range checks of SBP/DBP/PP values and
b. Variance of the cuff measurements vs the population BP Variance distribution model.
4. Calculate the BP calibration using the captured cuff and camera measurements.
5. Save the calculated BP calibration with BP-API.

3.1.1.1.1 Check Cuff Measurements

Prior to processing the user-entered cuff measurements each measurement is checked to ensure that it is a reasonable range of values. Values outside this range are considered a human or calibration-measurement error and will produce an error response without further calibration processing. The cuff measurement checks can include: SBP/DBP/PP cuff measurement range checks; and Paired cuff measurement population variance distribution checks.

3.1-1.1.1.1 Check Cuff Measurement Range

Prior to checking and filtering cuff measurements based on paired BP measurement variance the BP Calibration process checks if the recorded SBP and DBP cuff values and calculated PP value from these measurements is within an acceptable range. These are the same range checks for SBP/DBP/PP as is performed post-calculation by the BP Calculation process.

If SBP/DBP/PP range checks for any cuff measurements are violated, then an appropriate error is returned.

3.1.1.1.1.2 Check Paired Cuff Measurement Variance

Pairs of cuff measurements are generated from the 4 cuff measurements that make up the bracketed assessment, i.e. {(1,2), (2,3), (3,4)}.

The Population BP Variance Priors distribution model that is calculated as part of the BP Model training is loaded from S3. If there is an error loading this model the calibration process ends and an error is returned.

If successfully loaded the Population BP Variance Priors distribution model is applied to the difference between each pair of measurement's SBP, DBP, and PP values. This is based on the z-score of the distribution model and if any value of a pair fails a check of a z score>2 then the whole cuff measurement pair is removed from the calibration process. If all 3 pairs are removed, then a BP Calibration cannot be calculated, and the appropriate error is returned.

3.1.1.1.2 Calculate BP Calibration

With the SBP and DBP values of the calibrations cuff measurements and the processed PPG payloads of the camera-based measurements the process of calculating the user's BP calibration is as follows:

Compile a JSON dictionary with the key modelParams with the following fields: Compile a list of valid cuff measurements (i.e., cuff measurements that pass Population BP Variance Priors checks) SBP/DBP values; Compile a list of cuff measurements that failed Population BP Variance Priors checks (for debug); Calculate model-specific camera-based calibration parameters: wave_params (various dictionaries of internal signal quality and debug fields from PPG signal processing, beat segmentation, beat fitting, and filtering) and roots (various dictionaries of internal beat-fit fiducials used in conjunction with the user's BP calibration and BP model to calculate BP for this calculation).

3.1.2. BP Calculation Lambda

The purpose of BP Calculation process is to calculate the user's BP via signal processing of the user's PPG reading as recorded by the mobile device's camera and a previously establish BP calibration as calculated by the BP Calibration process. Before a BP calculation is performed the mobile client captures and validates, through on-device Quality Checks (QCs) the PPG signal from the user. Only after a signal of sufficient quality and duration is captured is the BP Calculation Lambda called by the SDK to initiate the calculation process.

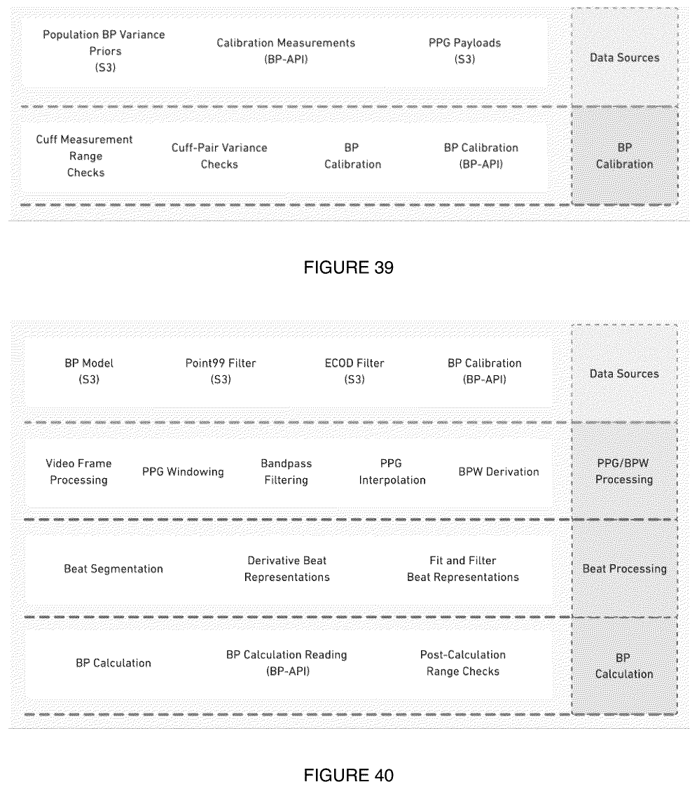

Example BP Calculation Lambda components are shown in FIG. 40.

3.1.2.1 BP Calculation Process

Upon routing of a BP Calculation endpoint request the BP Calculation process begins and can include the following sequential processing steps as detailed in the subsections that follow:
1. Load artifacts (models, filters) needed for BP calculation from S3.
2. Extract and process the recorded PPG signal(s) from the calculation Request to create a pre-filtered BPW signal.
3. Detect and segment heart beats on the BPW signal.
4. Detrend segmented beats and calculate beat signal power
5. Calculate the derivative representations of each detrended beat.
6. Filter based on signal power and beat correlation checks and fit derivative beats.
7. Load the user's BP calibration
8. Calculate the BP of remaining (valid) beats.
9. Store BP calculation with BP-API.
10. Perform post-calculation BP checks.

After successfully performing the post-calculation BP checks the BP Calculation Lambda's execution end with a Response returned to the SDK caller.

3.1.2.1.1 Load Artifacts (Model & Filters)

Artifacts can be loaded to calculate a BP measurement (e.g., BP Model —Main BP calculation model; Point99 Beat Filter—Statistical model of the 99^{th} percentile of expected beat-fit parameters used for beat-fit filtering; ECOD Filter—Beat filter model based on Empirical-Cumulative-Distribution-based Outlier Detection (ECOD) algorithm).

3.1.2.1.2 Extract and Process PPG

Multiple 15-sec PPG windows of signals can accompany a Request per the SDK design. As such the videoFrames key of the Request can have multiple window_<N> keys.

The first step in the PPG processing is to concatenate these windows of data to form a single PPG signal that will be used for BP calculation. Since on-device quality check models cause the data to be segmented in time the BP calculation pipeline will concatenate the PPG signals of all the provided windows: Subtract all i+1 signal window times by window i's ending time; Append the shifted PPG videoFrames of window i+1 to window I; Repeat until all videoFrames windows have been processed to create a signal representative PPG.

The single representative PPG signal is then further processed as follows: Invert the PPG to create a BPW representation of the signal (e.g., Note: This is done since low light levels in the camera recording represent high-pressure and high light levels in the camera recording represent low-pressure and it is the expected representation of beats for the BP model); Interpolate the PPG signal to 120 Hz; The BPW is filtered with a Butterworth IIR filter for the range of 0.5 Hz to 10 Hz.

The output of this processing step is a single, filtered BPW signal that is ready for beat segmentation.

3.1.2.1.3 Segment Beats

The band-pass filtered and BPW representation of the measurement PPG individual heart beats are detected and segmented using the following algorithm using slow and fast-moving averages: Calculate slow MA by convolving the band-passed PPG (sampled at 120 Hz) using window of 200 samples; Calculate the fast MA by convolving the band-passed filter using a window of 10 samples; Where the amplitude of the fast MA exceeds 3 times the amplitude of the slow MA indicates a potential beat start.

Returns error if no beats are detected during the beat segmentation processing.

3.1.2.1.4 Detrend Segmented Beats

Each segmented beat can be detrended using the following process: A slope calculated between the first element in the beat and the last element in the beat; The first element is set to zero; Each incremental element is detrended by subtracting the value of the slope at that elements point in time.

3.1.2.1.5 Check Detrend Beat Signal Power

Each detrended beat is checked for signal power using the following process: Elements are selected if their intensity is above two thresholds (4000 and 5000); The sum of all elements above each of these thresholds is calculated; Beats are checked for adequate raw intensity power by verifying that the sum of the two thresholds exceed according to the following equation: lower_threshold_auc+10*upper_threshold_auc>=100; Beats that do not pass this check are marked and ignored during blood pressure calculation.

If at least a threshold number of beats (e.g., 10, 12, 15, etc.) do not pass these checks, then no further processing is performed and an error is returned (e.g., returns error if fewer than a threshold number of beats (e.g., 10, 12, 15, etc.) valid beats remain after all filtering after beat power, correlation, and fit checks).

3.1.2.1.6 Calculate Derivate Beat Representations

The following derivative beat generation process is performed for each of the detected heartbeats:
1. A Savitzky-Golay (S-G) filter is used to filter data and generate derivative waveforms.
2. The raw time series data is filtered in its entirety
3. The filtered params are as follows:
   a. Twenty-one (21) sample moving window
   b. Third-order (cubic) basis functions
4. The first, second, and third derivatives are provided analytically by the S-G filtering process.
5. Each derivative is interpolated on a normalized time scale going from 0 to 1 and resampled to include 240 samples.
6. Each derivative is scaled using the dynamic range of the first derivative.
7. Further the second and third derivatives are scaled by the number of points in the time series (i.e., 240)
8. The derivative waveforms are segmented into beats using the beat markers found in Step (2)
9. Trapezoidal integration of the first derivative is used to recreate a de-meaned representation of the original beat waveform

3.1.2.1.7 Correlation Checks of the Second-derivative Beat Waveforms

The second derivative waveform for each beat is used to determine global and local correlation of all beats in the PPG signal and beat neighbors. Beats are deemed valid if their local correlation (to near neighbor beats) is high as well as their correlation with beats generally in the PPG signal.

3.1.2.1.8 Fit and Filter Derivative Beats

Each beat and its derivative representation are fit to BP model through independent BP Calculation beat-fit lambda calls. The output of the beat-fitting process is processed (fit) beats that are ready to be filtered.

The first filtering step is to confirm adequate fitting. This is done by checking the loss value of the objective function <=20.

The remaining filtering processes for beats can include 2 filtering steps. Filter processing is done over windows of 3 consecutive beats. These windows of beats from the Beat Segmentation step are created prior to filtering. Those 3-beat windows are then filtered by: Point99 Filtering—The various fiducial values of the beat fit process are checked for outliers (Z score>2) based on the trained population distribution; and Empirical cumulative distribution functions for outlier detection (ECOD) Filtering—Similar to Point99 filtering with a trained filter from the training dataset that is sensitive to the relationship between parameters.

If at least one 3-beat window is not filtered, then the BP calculation processing continues. Otherwise a filtering error is returned to the SDK as documented in the following subsection.

In a specific example, after all filtering checks a minimum of at least a threshold number of beats (e.g., 10, 12, etc.) is required to calculate average blood pressure change. Otherwise, a filtering error is returned to the SDK.

Returns error if fewer than the threshold number of valid beats (e.g., 10, 12, etc.) remain after all filtering and checks.

Returns error if there are errors compiling the fit and beat check arrays as part of the beat fitting process.

3.1.2.1.9 Calculate BP

Each 3-beat window that is not filtered by the Fit and Filter Derivate Beats process will have the SBP and DBP values estimated with the following BP calculation by applying the linear BP model loaded on startup to each of the parameters of the derivative representation of the fitted beats.

The average of each passed 3-beat window calculation constitutes the final systolic and diastolic BP calculation reading recorded in the BP-API and returned to the SDK caller in the Response.

### Illustrative Example 3

In a third illustrative example, the system and/or method can use all or portions of models described below.

1. Introduction

1.1 Acronyms and Abbreviations

| Acronym or | |
|---|---|
| Abbreviation | Description |
| API | Application Programming Interface |
| AWS | Amazon Web Services |
| BP | Blood Pressure |
| CNN | Convolutional Neural Network |
| DNN | Deep Neural Network |
| HAR | Human Activity Recognition |
| HF | Human Factors |
| iOS | Apple's iOS Operating System |
| ML | Machine Learning |
| PPG | Photoplethysmogram |
| QC | Quality Check |
| ReLU | Rectified Linear Unit |
| S3 | Amazon Simple Storage Service |
| SDD | Software Detailed Design |
| SDK | Software Development Kit |
| SRS | Software Requirement Specification |
| UI | User Interface |
| UX | User Experience |

1.2 Definitions

| Term | Definition |
|---|---|
| Binary Classifier | A classifier which categories elements into two groups, e.g. |
| | success/failure. |
| Blood Pressure | The force of circulating blood on the walls of the arteries. |
| | Blood pressure is taken using two measurements: systolic |
| | (measured when the heart beats, when blood pressure is at its |
| | highest) and diastolic (measured between heart beats, when |
| | blood pressure is at its lowest) |
| BP-ML | The collective system of software-part of BP Cloud- |
| | including the BP Calculation Lambda & BP Calibration |
| | Lambda. |
| Calibration | A set of features, derived from a sequence of cuff-based and |
| | camera-based readings, used to subsequently calculate a |
| | blood pressure. |
| Camera-Based Reading | A measurement taken using the camera on a mobile device, |
| | such as during a calibration procedure or blood pressure |
| | measurement, using the BP Monitor SDK. |
| Chroma | A representation of a video's color, often as a red and blue |
| | channel separate from the luma (black-and-white) portion of |
| | a color space |
| Core ML | An iOS framework to integrate machine learning models into |
| | applications. |
| Device Motion | A measure of how much a device is moving in space (e.g. |
| | acceleration, gravity, yaw, pitch). |
| Device Motion Model | An on-device machine learning model which detects if the |
| | Device Motion is within a required threshold. |
| Finger Detection/ | An on-device machine learning model which detects if a |
| Finger Detection Model | person's finger is on the camera lens, as a binary classifier. |
| Finger Guidance/ | An on-device machine learning model which detects where a |
| Finger Guidance Model | person's finger is on the camera lens, providing guidance for |
| | corrections as needed. |
| GitHub | A software source code control service. |
| Human Factors Models | On-device machine learned models that monitor device |
| | motion and user finger placement in order to obtain a high- |
| | quality PPG signal for processing. |
| Keras | An open-source software library that provides a Python |
| | interface and a higher-level abstraction for TensorFlow. |
| Luminance | A representation of the light intensity of a video frame's |
| | brightness and intensity, derived from the luma portion of a |
| | color space. |
| Machine Learning | A methodology of using algorithms and statistical models to |
| | analyze and draw inferences from patterns in data. |
| Photoplethysmogram | An optically-obtained plethysmogram that can be used to |
| (PPG) | detect blood volume changes in peripheral circulation. |
| BP Cloud | BP Cloud interfaces with the BP Monitor SDK installed on |
| | user Mobile Devices to facilitate blood pressure measurement |
| | sessions and to support other BP Monitor SDK related |
| | functionalities. |
| BP Monitor SDK | An embedded software package designed to run on user |
| | Mobile Devices that captures a PPG and provides a blood |
| | pressure measurement to the user. |
| BP-ML | The collective system of software-part of BP Cloud- |
| | including the BP Calculation Lambda & BP Calibration |
| | Lambda. |
| SDK User | A user of a 3rd Party application, which embeds the SDK. |
| TensorFlow | Open-source library for training machine learning models |
| | particularly Deep Neural Networks. |
| TFLight | (TensorFlow Light) A reduced size and faster format of a |
| | TensorFlow model. |
| Trainer | A user collecting data to be used for training a ML model. |
| User | The person using the SaMD. |
| Video Frame | An individual image frame within a contiguous stream of |
| | video data. |

2. System Overview

The software described in this document is an example of the architecture and implementation of the components used to specify and train a ML model for its specific classification purposes within the BP Monitor SDK.

In an example, the system can include 3 categories of components:
1) Preprocessing:
   a. Functions to download versioned training and test datasets stored on AWS S3
   b. Model-dependent functions to transform the annotated datasets to the input format of the ML model
   c. Training instance generation
2) Training:
   a. A definition of the ML model architecture
   b. Training hyperparameters
3) Postprocessing:
   a. Functions to evaluate the trained model with the test dataset
   b. Functions to export the model into a Core ML format compatible with the BP Monitor SDK
   c. Functions to upload a trained model and its evaluation to a versioned release destination on AWS S3

The output of exercising the software system described for each Human Factors model in this document is a trained and versioned ML model exported in the Core ML format that will be integrated with the BP Monitor SDK.

3. System Architecture

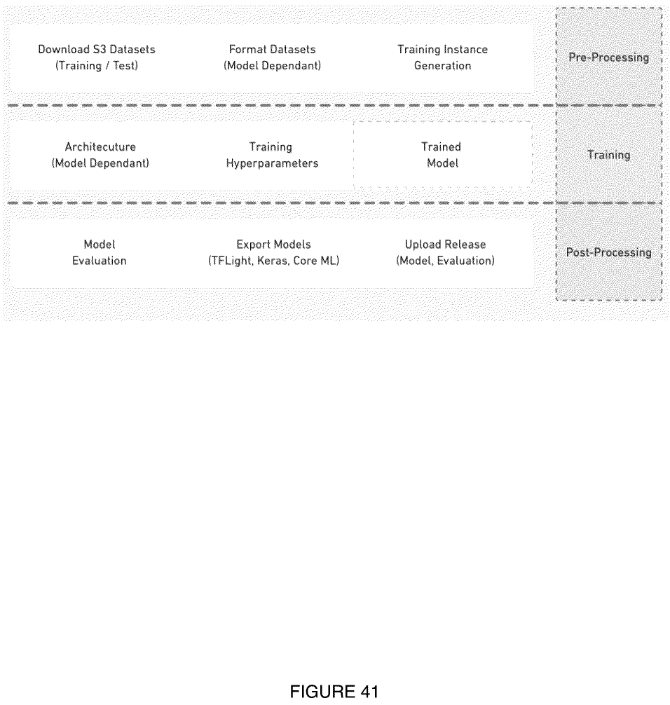

An example Human Factors Model architecture described in Section 2 is shown in FIG. 41.

The subsections that follow describe the processing components that can optionally be shared with all Human Factor Models, with model-dependent details documented in each model's specific subsection in Section 4.

3.1.1 Preprocessing

This section describes the optional common preprocessing aspects of on-device Human Factors models and specifics for each individual model.

3-1-1.1 Download S3 Datasets

Each model training configuration can specify a versioned, remote path on AWS from which versioned zipped training and test datasets are downloaded and processed using the following steps: Create a local temporary training directory per the model's name and dataset version; Download the remote versioned training and test datasets from AWS S3 using the Boto Python SDK; Unzip the local datasets.

3.1.1.2 Training Instance Generation

Training Instances are extracted from the captured recording. All Human Factors models operate on a classification window of 2-seconds worth of sampled data. However, any other classification window time period can be used. Although the width and sampling rate of each model's input data varies, the net sum of 2-seconds of data is submitted to each model for classification. This classification window represents a balance between the need to notify the user early of incorrect motion and/or finger position while also not notifying the user too often that measurements have to be restarted. A configuration parameter is defined in each training with a final defined period of 2-seconds as the agreed classification window with Mobile BP SDK.

Training data may be captured at a varied duration (e.g., from 2-seconds to 40-seconds). In order to further expand the training set beyond just the consecutive 2-second windows of data that was recorded from each user, the training code for Device Motion and Finger Detection utilizes sampling from the training set via 90% overlapping windows.

This technique is a way to increase the diversity of the training dataset using previously recorded data. The signals themselves are not augmented or processed in any way. Instead, new training samples are extracted from the existing recording by considering alternative window start times.

### Example: Sample Windowing

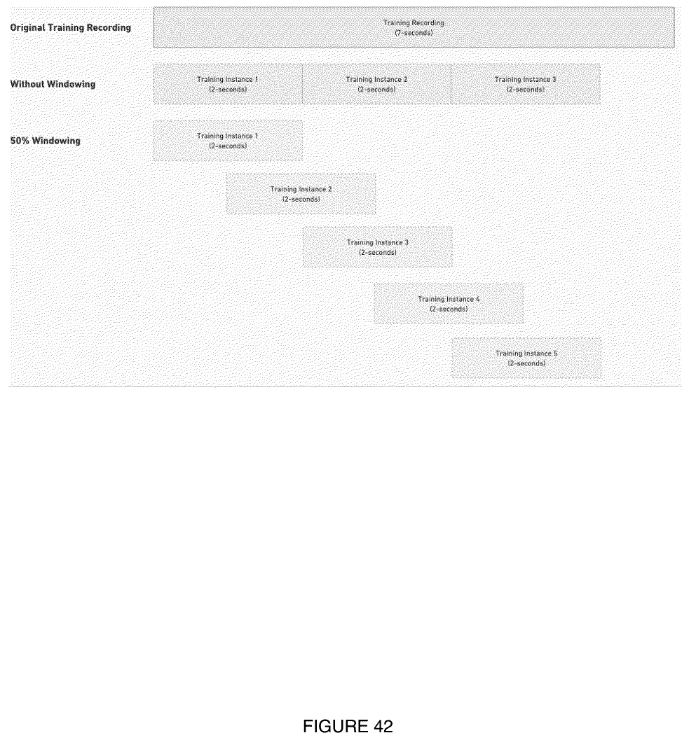

The “Without Window” scenario (e.g., example shown in FIG. 42) shows how training instances are extracted from an example training recording of 7 seconds. Without sample windowing, only 3 consecutive 2-second training instances would be extracted from the original recording. A complete 4^{th} instance is not available since there is an odd number of seconds in the training recording.

With an example 50% sample windowing, half of each training instance window of 2 seconds is reused for the next training instance. The window expands the sample dataset and trains the model to properly classify alternative views of the sensor data for the correct/incorrect cases being trained.

3.1.2 Training

These models can be trained ML models with the training data for each model including data recorded from a group of users for a variety of device motion and/or finger placement/guidance scenarios. Those datasets are resampled and expanded to create an even larger number of actual training and test instances as described in Section 3.1.2.1

Each DNN model described in this document is a TensorFlow defined and trained model using the Keras Functional Application Programming Interface (API). The development plan for these models is an iterative process of training and evaluation. Each model is tuned and trained for overall accuracy of classification as well as minimizing the overall size of the model per the details outlined in Section 3.1.2.1

3.1.2.1 Architecture and Hyperparameters

There are a number of Deep Neural Network (DNN) architectures (e.g. number and types of layers, activation functions, etc.) and hyperparameters (e.g. epochs, batch sizes, loss functions, optimizers, etc.) that can implement the intended purpose of a model. During development the DNN architecture and hyperparameters are selected based on analysis of training evaluation and with the following high-level goals and approaches:
Provide an accurate model based on training and holdout validation dataset accuracy (e.g. model generalization)
   Introduce layers (e.g. BatchNormalization) and training techniques (e.g. early stopping, dropout) that promote generalization.
Minimize the complexity of the model (e.g. training parameters)
   Minimize the number of layers.
   Minimize the complexity and width of layer.
Minimize the training duration (e.g. epochs) which can lead to overfitting.

Details of examples of the final network architecture and training hyperparameters are documented in each Human Factor model's specific subsection in Section 4.

3.1.3 Postprocessing

This section describes the optional common postprocessing aspects of all on-device Human Factors models and specifics for each individual model.

3.1.3.1 Model Evaluation

In addition to the built-in accuracy metrics from the TensorFlow training process, there are standalone evaluation methods that are called after training which process the training and test datasets with the final trained model to produce the following scores to include in the model evaluation report: Binary Classification (Precision, Recall, F1, AUC, and Accuracy) and Multiclass Classification (Accuracy).

3.1.3.2 Export Models

The output of the model training process can be a Keras (.h5) model. Exporting that model just involves saving it to the local versioned training output directory. The Keras model is then passed through a TFLiteConverter that is built into TensorFlow and that model is also saved to the versioned training output directory. Finally, the Keras model is also converted to iOS Core ML format using the Core ML Tools library. Each model's specific Core ML export function also annotates the input/output definitions so that the binary that is included with the BP Monitor SDK is properly documented.

3.1.3.3 Upload Release

Based on each model training configuration specified, a versioned release is uploaded to AWS S3 which includes: Keras/TFLite/Core ML model binaries; Model evaluation; Complete training log.

4 Human Factors Models

The subsections that follow describe examples of the purpose, architecture, and implementation of 3 human factors models that can be part of the BP Monitor SDK.

4.1 Device Motion Model

4.1.1 Overview

The purpose of the on-device Device Motion model is to flag improper device and/or user motion that would lead to an incorrect and/or suboptimal PPG measurement. Proper blood pressure measurement requires a user be seated and at rest. The Device Motion ML model uses various device motion sensors that are programmatically accessible via motion SDKs available through iOS for the purpose of Human Activity Recognition (HAR).

Classification decisions from this model are used to alter the user's experience flow in the BP Monitor SDK by notifying the user so they can adjust their body position and/or device motion to complete an accurate PPG measurement.

4.1.2 Architecture

An example of the Device Motion DNN model is detailed in the following sections.

4.1.2.2 Layers

The Device Motion model can be a trained Convolutional Neural Network (CNN) that has 3 sections of layers: Input—A single, exposed layer that is driven by samples of independent variables to be classified; Convolution—Hidden layers that learn the features for classification from the time-series data across the sample window; Classification—The feed-forward layer of the network that learn to classify the convolutional representation of the input data and whose final layer outputs the dependent variable, i.e. classification decision.

4.1.2.2.1 Input Layer

The movement of the user/device is captured via on-device sensors sampled at 60 Hz (samples/second) and classified over an accumulated 2-second window of measurements for a total of 120 samples per classification. There are 12 sensor input channels for 4 different categories of motion sensing that makeup the Input Layer of the network:
Gravity (gravity_x/gravity_y/gravity_z)—The gravity acceleration vector expressed in the device's reference frame.
   Each channel is represented as a 2D float32 array of the shape [120×1]
Acceleration (acceleration_x/acceleration_y/acceleration_z)—The device's acceleration vector expressed in the device's reference frame.
   Each channel is represented as a 2D float32 array of the shape [120×1]
Rotation (rotation_rate_x/rotation_rate_y/rotation_rate_z)—The device's rotation-rate vector expressed in the device's reference frame.
   Each channel is represented as a 2D float32 array of the shape [120×1]
Attitude (attitude_pitch/attitude_roll/attitude_yaw)—The device's attitude position vector expressed in the device's reference frame.
   Each channel is represented as a 2D float32 array of the shape [120×1]

4.1.2.2.2 Convolution Layer

The convolutional layer of the Device Motion model can optionally contain the following layers for the purposes of learning spatial features in the 2D (time-series) data signal that makes up the Device Motion input channels. In this CNN the features are learned jointly for the combined (concatenated) representation of the input signals. A 1D version of each CNN layer is used given the nature of the time-series input data being operated on. Any parameters not specified are TensorFlow (v2.7.0) defaults.
Concatenate—To start the 12 input channels of the Device motion model's Input Layer are concatenated to create a single (1,440 wide) vector representation of the input feature space.
BatchNormalization—Training is done in mini-batches of the training data set and the concatenated input data is normalized for each mini-batch. Batch normalization is a standard technique in Neural Network (NN) training to standardize the input ranges of variables which leads to stability and faster convergence in training.
SeparableConv1D—The configuration of the 1D convolution is as follows: 8 filters, kernel size of 3, with same padding a ReLU activation function. A Separable form of convolution is used for the advantage of computational speed from separating the convolution calculation without altering the effectiveness vs a normal Conv1D layer.
SpatialDropout1D—A dropout layer is used to randomly exclude a NN variable/weight from the training dataset with a rate of 50% (0.5). This common NN training regularization technique promotes generalization in the training as individual variable weights won't have as many training cycles to be present and overfit. Furthermore, a Spatial version of this layer is used to allow dropout of entire feature maps from the previous convolutional layer instead of single variable's weight further preventing co-adaptation of weights among variables within a feature map from compensating for individual missing weights.
MaxPooling1D—Down sampling of the feature maps of the convolution layer is performed using Max pooling (pool size of 8) whereby the maximum value is sampled from each feature map.
Flatten—The output of the MaxPooling1D layer is flatten to a size of 1,440 to prepare for the Feed-Forward section of the network that is responsible for the classification task.

4.1.2.2.3 Classification Layer

The Dense intermediate layer and Dense output layer make up the overall classification layer:
Dense—The intermediate layer (2 units wide) uses a non-linear ReLU activation.
Dense—The output layer (2 units wide) uses a SoftMax activation corresponding to the model's overall 2-bit output vector.
The output of the model is a 2-bit motion decision vector (motion_decision) with the following one-hot encoding.
   [0,1]—Correct Motion—Measurement process can proceed with no motion objection.
   [1,0]—Incorrect Motion—Measurement process should be interrupted with a motion objection.

4.1.2.2.4 Example Device Motion Decision

The weight for each output encoding will be given as a percentage, with the overall weight of all encoding values for a given prediction adding up to 1.0. The position with the maximum weight shall be taken as the prediction. For example, [0.25, 0.75] is considered a Correct Motion prediction with 75% confidence.

4.1.3 Preprocessing

Example Device Motion model-specific preprocessing functions are described in the following subsections.

4.1.3.1 Format Dataset

The training and test datasets are reformatted to meet the Input layer architecture specified in Section 4.1.2.2.1. The first step of processing is to extract the sensor and timestamp data from the deviceMotion key of each dataset file. The training instance windowing described in Section 3.1.1.2 is then applied get an expanded, 2-second window representation of the training/test datasets across all 12 motion sensors (gravityX, gravityY, gravityZ, accelerationX, accelerationY, accelerationZ, rotationRateX, rotationRateY, rotationRateZ, attitudePitch, attitudeRoll, attitudeYaw). The training/test instance binary label is extracted from the file name and collected alongside the training instance.

After this dataset processing step the training and test instances are properly formatted for the model training and post-training model evaluation steps, respectively.

4.1.4 Training

4.1.4.1 Architecture

An example DNN model architecture is described in detail in Section 4.1.2.

4.1.4.2 Training Hyperparameters

The training hyperparameters for the Device Motion Detection DNN model are as follows: #Epochs—10; Batch Size—32; Loss Function—Binary Cross Entropy; Optimizer—Adam.

4.2 Finger Detection Model

4.2.1 Overview

The purpose of the on-device Finger Detection model is to flag improper finger position by the user on the device's measurement camera. Improper and/or non-ideal finger placement on the camera could lead to an incorrect and/or suboptimal PPG measurement. The Finger Detection ML model uses a summed luminescence value and total luminescence of the red and blue channels of the video signal via the measurement camera on the device.

Classification decisions from this model are used to alter the user experience flow in the BP Monitor SDK by notifying the user so they can adjust their finger position to complete an accurate PPG measurement.

4.2.1.1 Relation to Finger Guidance Model

The Finger Detection model serves a similar purpose as the Finger Guidance model (See Section 4.3) except that it utilizes a different representation of the user's finger position (i.e. total frame luminescence and total red/blue chroma luminescence). Total frame luminescence is the primary signal representing the user's PPG from which BP measurement with BP Cloud is based. Therefore, the Finger Detection model detects the fundamental PPG signal that the Finger Guidance model cannot.

The overall Finger Detection decision for the BP Monitor SDK is therefore a logical AND of the output of the Finger Guidance and Finger Detection models: finger_detected=finger_detect_decision AND finger_guidance_decision

4.2.2 Architecture

An example Finger Detection DNN model is detailed in the following sections.

4.2.2.2 Layers

The Finger Detection model can be a trained Convolutional Neural Network (CNN) that has 3 sections of layers: Input—A single, exposed layer that is driven by samples of independent variables to be classified; Convolution—Hidden layers that learn the features for classification from the time-series data across the sample window; Classification—The feed-forward layer of the network that learns to classify the convolutional representation of the input data and whose final layer outputs the dependent variable, i.e. classification decision.

4.2.2.2.1 Input Layer

A stream of video frames recorded from the device's camera is captured using a set of verified device-specific camera settings (resolution, framerate, ISO, exposure, etc.) as reported in the Camera Module specification over an accumulated 2-second window of measurements for a total of 120 samples per classification. There are 3 video input channels:
Luminance Intensity (luminance_intensity)—The sum total luminescence of all video frame channels.
   Each channel is represented as a 2D float32 array of the shape [120×1]
Chroma Red Intensity (chroma_red_intensity)—The sum total luminescence of the red chroma video frame channels.
   Each channel is represented as a 2D float32 array of the shape [120×1]
Chroma Blue Intensity (chroma_blue_intensity)—The sum total luminescence of the blue chroma video frame channels.
   Each channel is represented as a 2D float32 array of the shape [120×1]

4.2.2.2.2 Convolution Layer

There can be separate convolutional layers for each of the 3 input channels of the Finger Detection model. Each contains the following layers for the purposes of independently learning spatial features in the 2D (time-series) data signal that makes up the Finger Detection input channels. Learned representations of each channel are combined (concatenated) as a feature representation for classification. 1D version of each CNN layer is used given the nature of the time-series input data being operated on. Any parameters not specified are TensorFlow (v2.7.0) defaults.
BatchNormalization—Training is done in mini-batches of the training data set and the concatenated input data is normalized for each mini-batch. Batch normalization is a standard technique in Neural Network (NN) training in order to standardize the input ranges of variables which leads to stability and faster convergence in training. A momentum value of 0.9 is used to account for previous mini batches of normalization.
SeparableConv1D—The configuration of the 1D convolution is as follows: 64 filters, kernel size of 3, with same padding a ReLU activation function. A Separable form of convolution is used for the advantage of computational speed from separating the convolution calculation without altering the effectiveness vs a normal Conv1D layer.
SpatialDropout1D—A dropout layer is used to randomly exclude a NN variable/weight from the training dataset with a rate of 50% (0.5). This common NN training regularization technique promotes generalization in the training as individual variable weights won't have as many training cycles to be present and overfit. Furthermore, a Spatial version of this layer is used to allow dropout of entire feature maps from the previous convolutional layer instead of single variable's weight further preventing co-adaptation of weights among variables within a feature map from compensating for individual missing weights.
MaxPooling1D—Down sampling of the feature maps of the convolution layer is performed using Max pooling (pool size of 2) whereby the maximum value is sampled from each feature map.
Flatten—The output of the MaxPooling1D layer is flattened to prepare for the Feed-Forward section of the network that is responsible for the classification task.
Concatenate—The output of each channel's learned features from their CNN layers is combined (concatenated) to create a single vector representation (23,040 variables wide) of the overall features for classification.

4.2.2.2.3 Classification Layer

The Dense intermediate layers and Dense output layer make up the overall classification layer:
Dense—The intermediate layers (32 and 16 units wide, respectively) use a non-linear ReLU activation.
Dense—The output layer (2 units wide) uses a SoftMax activation corresponding to the model's overall 2-bit output vector.
The output of the model is a 2-bit finger-detection decision vector (finger_detection_decision) with the following one-hot encoding:
   [0,1]—Finger Detected—valid PPG detected—Measurement process can proceed with a finger properly detected on the camera.
   [1,0]—Finger Not Detected—no valid PPG detected—Measurement process should be interrupted with a finger not detected objection.

4.2.2.2.4 Example Finger Detection Decision

The weight for each output encoding will be given as a percentage, with the overall weight of all encoding values for a given prediction adding up to 1.0. The position with the maximum weight shall be taken as the prediction. For example, [0.25, 0.75] is considered a Finger Detected prediction with 75% confidence.

4.2.3 Preprocessing

Example Finger Detection model-specific preprocessing functions are described in the following subsections.

4.2.3.1 Format Dataset

The training and test datasets are reformatted to meet the Input layer architecture specified in Section 4.2.2.2.1. The first step of processing is to extract the video channel and timestamp data from the videoFrames key of each dataset file. The training instance windowing described in Section 3.1.1.2 is then applied to get an expanded, 2-second window representation of the training/test datasets across all 3 video channels (luminanceIntensity, chromaRedIntensity, chromaBlueIntensity). The training/test instance binary label is extracted from the file name and collected alongside the training instance.

After this dataset processing step the training and test instances are properly formatted for the model training and post-training model evaluation steps, respectively.

4.2.4 Training

4.2.4.1 Architecture

An example DNN model architecture is describe in detail in Section 4.2.2.

4.2.4.2 Training Hyperparameters

Example training hyperparameters for the Finger Detection DNN model are as follows: #Epochs—10; Batch Size—128; Loss Function—Binary Cross Entropy; Optimizer—Adam.

4.3 Finger Guidance Model

4.3.1 Overview

The purpose of the on-device Finger Guidance model is to flag improper finger position by the user on the device's measurement camera. Improper and/or non-ideal finger placement on the camera could lead to an incorrect and/or suboptimal PPG measurement. The Finger Guidance ML model uses of an array of summed row and column intensities (total luminescence) of the video signal via the measurement camera on the device.

Classification decisions from this model are used to alter the user experience flow in the BP Monitor SDK by notifying the user so they can adjust their finger position to complete an accurate PPG measurement.

4.3-11 Relation to Finger Detection Model

The Finger Guidance model serves a similar purpose as the Finger Detection model (See Section 4.2) except that it utilizes a different representation of the user's finger position (i.e. row+column luminescence). This allows the Finger Guidance model to detect inappropriate and/or non-ideal finger placement that the Finger Detection model may miss—namely the case where the user has their finger mostly on the torch on the back of the device instead of on the camera.

The PPG intensity of such a finger placement can appear in the inputs associated with the Finger Detection model (i.e., total luminescence and total red/blue chroma luminescence) to be a valid PPG signal. The Finger Guidance's inputs, however, can detect this case as a non-ideal placement.

The overall Finger Detection decision for the BP Monitor SDK is therefore a logical AND of the output of the Finger Guidance and Finger Detection models: finger_detected=finger_detect_decision AND finger_guidance_decision

4.3.2 Architecture

An example Finger Guidance DNN model is detailed in the following sections.

4.3.2.2 Layers

The Finger Guidance model can be a trained Convolutional Neural Network (CNN) that has 3 sections of layers: Input—A single, exposed layer that is driven by samples of independent variables to be classified; Convolution—Hidden layers that learn the features for classification from the time-series data across the sample window; Classification—The feed-forward layer of the network that learn to classify the convolutional representation of the input data and whose final layer outputs the dependent variable, i.e. classification decision.

4.3.2.2.1 Input Layer

The finger position of the user is captured via an unfiltered stream of video frames recorded from the device's camera captured using a set of verified device-specific camera settings (resolution, framerate, ISO, exposure, etc.) as reported in Camera Module specification over an accumulated 2-second window of measurements at 120 frames-per-second for a total of 240 samples per classification. There are 2 video input channels, recorded with the device camera in a portrait orientation:
Row Luminance Intensity (row_intensities)—sum over each row, representing the height of the video frame image buffer
   Each channel is represented as a 2D float32 array of the shape [240×1280]
Column Luminance Intensity (col_intensities)—sum over each column, representing the width of the video frame image buffer
   Each channel is represented as a 2D float32 array of the shape [240×720]

4.3.2.2.2 Convolution Layer

There can be separate convolutional layers for each of the 2 input channels of the Finger Guidance model. Each contains the following layers for the purposes of independently learning spatial features in the 2D (time-series) data signal that makes up the Finger Guidance input channels. Learned representations of each channel are combined (concatenated) as a feature representation for classification. 1D version of each CNN layer is used given the nature of the time-series input data being operated on. Any parameters not specified are TensorFlow (v2.7.0) defaults.
BatchNormalization—Training is done in mini-batches of the training data set and the concatenated input data is normalized for each mini-batch. Batch normalization is a standard technique in Neural Network (NN) training in order to standardize the input ranges of variables which leads to stability and faster convergence in training. A momentum value of 0.9 is used to account for previous mini-batches of normalization.
SeparableConv1D—The configuration of the 1D convolution is as follows: 32 filters, kernel size of 3, with same padding a ReLU activation function. A Separable form of convolution is used for the advantage of computational speed from separating the convolution calculation without altering the effectiveness vs a normal Conv1D layer.
SpatialDropout1D—A dropout layer is used to randomly exclude a NN variable/weight from the training dataset with a rate of 50% (0.5). This common NN training regularization technique promotes generalization in the training as individual variable weights won't have as many training cycles to be present and overfit. Furthermore, a Spatial version of this layer is used to allow dropout of entire feature maps from the previous convolutional layer instead of single variable's weight further preventing co-adaptation of weights among variables within a feature map from compensating for individual missing weights.
MaxPooling1D—Down sampling of the feature maps of the convolution layer is performed using Max pooling (pool size of 2) whereby the maximum value is sampled from each feature map.
Flatten—The output of the MaxPooling1D layer is flattened to prepare for the Feed-Forward section of the network that is responsible for the classification task.
Concatenate—The output of each channel's learned features from their CNN layers is combined (concatenated) to create a single vector representation (7,680 variables wide) of the overall features for classification.

4.3.2.2.3 Classification Layer

The Dense intermediate layer and Dense output layer make up the overall classificationlayer:
Dense—The intermediate layer (128 units wide) uses a non-linear ReLU activation.
Dense—The output layer (2 units wide) uses a SoftMax activation corresponding to the model's overall 2-bit output vector.
The output of the model is an 8-bit finger-guidance decision vector (finger_guidance_decision) with the following one-hot encoding:
   Correct—Measurement process can proceed with a finger properly detected on the camera.
      [1,0,0,0,0,0,0,0]—Ideal Placement—No Guidance
   Incorrect—Measurement process should be interrupted, and the user offered guidance to adjust their finger placement and restart measurement.
      [0,1,0,0,0,0,0,0]—Decrease Finger Pressure—Finger is on camera but with too much pressure.
      [0,0,1,0,0,0,0,0]—Increase Finger Pressure—Finger is hovering over camera without enough pressure.
      [0,0,0,1,0,0,0,0]—Shift Finger Up—Finger is not centered (top of lens exposed)
      [0,0,0,0,1,0,0,0]—Shift Finger Down—Finger is not centered (bottom of lens exposed)
      [0,0,0,0,0,1,0,0]—Shift Finger Left—Finger is not centered (left-side of lens exposed)
      [0,0,0,0,0,0,1,0]—Shift Finger Right—Finger is not centered (right-side of lens exposed)
      [0,0,0,0,0,0,0,1]—Stop Moving Finger—Finger is Sliding/Rolling (Up/Down/Left/Right) or tapping on camera.

4.3.2.2.4 Example Finger Guidance Decision

The weight for each output encoding will be given as a percentage, with the overall weight of all encoding values for a given prediction adding up to 1.0. The position with the maximum weight shall be taken as the prediction. For example, [0.75, 0, 0, 0.20, 0, 0, 0.05, 0] is considered an Ideal Placement prediction with 75% confidence.

4.3.3 Preprocessing

Example Finger Guidance model-specific preprocessing functions are described in the following subsections.

4.3.3.1 Format Dataset

The training and test datasets can be reformatted to meet the Input layer architecture specified in Section 4.3.2.2.1. The first step of processing is to extract the video channel and timestamp data from the videoFrames key of each dataset file. The training instance windowing described in Section 3.1.1.2 is then applied get an expanded, 2-second window representation of the training/test datasets across both video channels (rowIntensities, columnIntensities). The training/test instance binary label is extracted from the file name and collected alongside the training instance.

After this dataset processing step the training and test instances are properly formatted for the model training and post-training model evaluation steps, respectively.

4.3.4 Training

4.3.4.1 Architecture

An example DNN model architecture is describe in detail in Section 4.3.2.

4.3.4.2 Training Hyperparameters

The training hyperparameters for the Finger Guidance DNN model are as follows: #Epochs—100; Batch Size—128; Loss Function—Categorical Cross Entropy; Optimizer—Adam.

Different subsystems and/or modules discussed above can be operated and controlled by the same or different entities. In the latter variants, different subsystems can communicate via: APIs (e.g., using API requests and responses, API keys, etc.), requests, and/or other communication channels.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A method, comprising:
using an image sensor, sampling a set of images of a body region of a user;
determining a plethysmogram (PG) dataset based on the set of images;
using a trained model, determining a placement of the body region relative to the image sensor based on a set of attributes extracted from the set of images;
processing the PG dataset in response to detecting that a set of criteria for the placement of the body region are satisfied, wherein processing the PG dataset comprises:
segmenting the PG dataset into segments;
for each of the segments, determining a signal quality for the segment; and
determining a subset of the segments associated with a signal quality that satisfies a signal quality criterion; and
determining a cardiovascular parameter based on the subset of segments.

2. The method of claim 1, wherein detecting that the set of criteria for the placement of the body region are satisfied comprises at least one of: detecting contact between the body region and the image sensor, detecting an acceptable placement of the body region on the image sensor, detecting an acceptable contact pressure between the body region and the image sensor, or detecting an acceptable level of body region motion.

3. The method of claim 1, wherein the cardiovascular parameter is determined in response to detecting that greater than a threshold number of segments are associated with a signal quality that satisfies the signal quality criterion, the method further comprising, in response to detecting that less than the threshold number of segments are associated with a signal quality that satisfies the signal quality criterion, guiding the user to adjust a temperature of the body region.

4. The method of claim 3, wherein the threshold number of segments is at least 10.

5. The method of claim 1, wherein, for each of the segments: the signal quality for the segment comprises a signal power metric, wherein the signal quality for the segment satisfies the signal quality criterion when the signal power metric is greater than a threshold.

6. The method of claim 1, wherein, for each of the segments: the signal quality for the segment comprises a local correlation metric and a global correlation metric, wherein the signal quality for the segment satisfies the signal quality criterion when the local correlation metric is greater than a first threshold and the global correlation metric is greater than a second threshold.

7. The method of claim 6, wherein, for each of the segments: determining the signal quality for the segment comprises determining a second derivative of the segment and calculating the local correlation metric and the global correlation metric based on the second derivative.

8. The method of claim 1, further comprising, for each of the segments: fitting a fiducial model to the segment and to a first derivative of the segment, wherein the signal quality for the segment is determined based on a loss for the fitted fiducial model; wherein the cardiovascular parameter is determined based on the fiducial models corresponding to the subset of segments.

9. The method of claim 1, further comprising, for each of the segments: fitting a fiducial model to the segment and to a first derivative of the segment, wherein the signal quality for the segment is determined based on fit parameters for the fiducial model; wherein the cardiovascular parameter is determined based on the fit parameters for the fiducial models corresponding to the subset of segments.

10. The method of claim 9, wherein the signal quality for the segment is further determined based on fit parameters for a fiducial model fit to an adjacent segment.

11. The method of claim 1, wherein the set of attributes comprises the PG dataset.

12. The method of claim 1, wherein each segment corresponds to a heartbeat.

13. A system, comprising:
a processing system configured to:
receive a set of images of a body region of a user, the set of images sampled by an image sensor;
determine a plethysmogram (PG) dataset based on the set of images;
using a first model, determine a placement of the body region relative to the image sensor based on the set of images, wherein the model is trained using sets of training images, each set of training images corresponding to a time window, wherein at least a portion of the time windows comprise overlapping time windows;
in response to detecting that a set of criteria for the placement of the body region are satisfied, determine a signal quality for the PG dataset using a second model; and
in response to detecting that the signal quality satisfies a signal quality criterion, determine a cardiovascular parameter based on the PG dataset.

14. The system of claim 13, wherein the processing system comprises a remote processing system and a local processing system on a user device, wherein the placement of the body region relative to the image sensor is determined using the local processing system, wherein the signal quality for the PG dataset is determined using the remote processing system.

14. The system of claim 13, wherein the placement of the body region relative to the image sensor comprises a confidence score for each of a set of placement classifications.

15. The system of claim 14, wherein the placement classifications comprise: proper placement, improper placement associated with a first direction, and improper placement associated with a second direction.

16. The system of claim 13, further comprising, in response to detecting that the signal quality does not satisfy the signal quality criterion, guiding the user to increase a temperature of the body region.

17. The system of claim 13, wherein the first model comprises a convolutional neural network.

19. The system of claim 13, wherein the cardiovascular parameter comprises at least one of a blood pressure or a heart rate.

20. The system of claim 13, wherein the cardiovascular parameter is displayed at a user device.
